(19) United States
(12) Patent Application Publication
(10) Pub. No.: US 2017/0028176 A1
(43) Pub. Date: Feb. 2, 2017

(54) TRANSLUMINAL IMPLANT AND METHODS AND APPARATUS FOR LOADING, DELIVERING, AND DEPLOYING AN IMPLANT

(71) Applicant: Treus Medical, Inc., REDWOOD CITY, CA (US)

(72) Inventors: Jacques Van Dam, LOS ANGELES, CA (US); James Craig Milroy, PALO ALTO, CA (US); Robert Matthew Ohline, REDWOOD CITY, CA (US); John Patrick Lunsford, LOS ALTOS HILLS, CA (US); Stacy Patch, SAN JOSE, CA (US)

(21) Appl. No.: 14/810,231

(22) Filed: Jul. 27, 2015

(51) Int. Cl. A61M 27/00; A61M 39/22; A61B 8/12

(52) U.S. Cl. CPC A61M 27/002; A61B 8/12; A61M 39/22; A61M 2210/1075

(57) ABSTRACT

A device for implantation in a body may include a body defining a lumen. The body may have a first end and a second end, and the lumen may define a central axis. The body may include a first flexible flange having a first base portion and a first angled portion. The first base portion may be about perpendicular to the central axis and extend radially away from the first end. The body may further include a second flexible flange having a second base portion and a second angled portion. The second base portion may be about perpendicular to the central axis and extend radially away from the second end. The first and second angle portions may be generally angled toward each other. A loading device may be configured to load the device into a delivery device. The delivery device may be configured to delivery and deploy the device.

### BACKGROUND

Endoscopic ultrasound (EUS) may provide endoscopists with a powerful imaging modality, enabling visualization of and access to tissue beyond the walls of certain areas or organs of the body, such as the gastrointestinal tract, without the use of ionizing radiation. EUS has primarily been used for diagnostic procedures, such as taking biopsies and staging tumors, as well as certain therapeutic procedures. Using EUS within therapeutic procedures may improve patient care, reduce procedure risk, and reduce cost.

One therapeutic procedure that may utilize EUS involves creating communication between two existing lumens or cavities within the body that are accessible with an echo-endoscope. For example, the small intestine may be connected to the gallbladder. These therapeutic procedures may be used to treat a variety of diseases, including but not limited to biliary disease, pancreatic pseudocysts, and obstructions of the gastrointestinal tract caused by tumors. Biliary disease, for example, often results in cholecystectomy, which is the most frequently performed abdominal surgery in the United States.

The primary objective of these therapeutic procedures is to create a junction to allow material to flow between two lumens. The junction must be secure, stable, patent, and leak-proof, as well as must remain viable indefinitely. Typically, the junction is created by means of surgical anastomosis in an open procedure (e.g. cholecystodoudenostomy). Alternatively, the junction may be created endoscopically, through the use of standard indefinitely indwelling pigtail catheters, naso-biliary tubes, and self-retaining shunts.

However, both approaches to creating the junction have deficits and fail to address major risks in the procedure. For example, the surgical approach is a major, highly invasive, open procedure and is therefore only rarely performed. The endoscopic approach is technically challenging, is vulnerable to complications resulting from migration, leaks, and long-term viability, and requires additional follow-up procedures to maintain or remove any implanted devices.

Further, endoscopic delivery of self-retaining shunts may be particularly difficult since the shunts may not be delivered through the working channel of a typical ultrasound or standard endoscope.

### SUMMARY

According to one embodiment, a device for implantation in a body may include a body defining a lumen. The body may have a first end and a second end, and the lumen may define a central axis. The body may include a first flexible flange having a first base portion and a first angled portion. The first base portion may be generally or about perpendicular to the central axis and extend radially away from the first end. The body may further include a second flexible flange having a second base portion and a second angled portion. The second base portion may be about perpendicular to the central axis and extend radially away from the second end. The first and second angle portions may be generally angled toward each other.

According to another embodiment, a medical apparatus for forming a shunt between two tissues may include a tube having a first end and a second end, a first tissue engager may have a first aperture, and a second tissue engager may have a second aperture. The first aperture may be fitted to the first end of the tube, and the second aperture may be fitted to the second end of the tube. The tube, first tissue engager, and second tissue engager may form an object capable of receiving tissue between the first and second tissue engagers.

According to another embodiment, a method of folding a device may include expanding a central aperture of the device. The central aperture may include a first flexible flange and a second flexible flange. The method may further include extending the first flange and the second flange away from each other and radially compressing the first flange and the second flange into a pleated configuration toward an axis extending through a center of the central aperture. The method may further include compacting the device in the pleated configuration such that a diameter of the device is equal to or less than an inner diameter of a loading mechanism and inserting a tube within the central aperture.

According to yet another embodiment, a method of loading a compressed implant into a delivery device may include aligning a central aperture of the compressed implant with a central lumen of the delivery device, wherein the delivery device may include an implant positioner at least partially within a capsule. The method may further include advancing the implant positioner at least partially out from the capsule toward the compressed implant, inserting a first flange of the compressed implant into the implant positioner, and retracting the implant positioner with the compressed implant into the capsule.

According to yet another embodiment, a method of packaging and deploying an implant may include extending the implant along a lengthwise direction of the implant, radially compressing the implant along the lengthwise direction, and inserting a first flange of the implant into an implant holder on an end of a deployment device. The method may further include retracting the implant holder and implant into the deployment device, advancing the implant holder toward the end of the delivery device, and releasing a second flange out from the end of the deployment device. The method may further include advancing the implant holder at least partially out from the end and releasing the first flange from the deployment device and the implant holder.

According to still another embodiment, a method of deploying a device within a patient may include delivering the device in a compressed configuration with a deployment mechanism at least partially through a first opening to a first cavity within the patient and releasing a second flange of the device within the first cavity. The method may further include retracting the deployment mechanism back through the first opening, wherein a first flange of the device is locatable within a second cavity and releasing the first flange of the device within the second cavity.

According to another embodiment, a method of deploying an implant from a delivery device may include advancing an implant positioner toward an end of the delivery device, wherein the implant maybe secured in a compressed configuration within the implant holder. The method may further include releasing a second flange of the implant through the end, advancing the implant holder at least partially out of the end, and releasing a first flange of the implant from the implant positioner.

According to yet another embodiment, a delivery device configured to deploy a shunt within a patient may include a generally cylindrical container configured to retain the shunt in a compressed configuration and a positioning mechanism movable between a first position and a second position within the container. The positioning mechanism may be within the container in the first position and may be at least partially extended beyond a distal end of the container in the second position. The delivery device may further include a control device configured to move the positioning mechanism between the first position and the second position and a lumen connecting a proximal end of the container to the control device and extendable into the patient. The control device may manipulate the positioning mechanism through the lumen.

According to still another embodiment, a loading device for folding and loading a device into a delivery device may include a dilator configured to expand a lumen of the device, wherein the lumen defines a central axis of the device and a compressor configured to radially compress the device along a central axis. The device may assume a pleated configuration with the compressor. The loading device may further include a loading tool configured to receive the implant in the pleated configuration and radially compress the implant into a compressed configuration. The loading tool may be attachable and alignable with the delivery device.

### BRIEF DESCRIPTION OF THE FIGURES

Features, aspects, and advantages of the present invention will become apparent from the following description, appended claims, and the accompanying exemplary embodiments shown in the drawings, which are briefly described below.

FIGS. 1A-1D are perspective, cross-sectional side, cross-sectional perspective, and top views, respectively, of an implant according to one embodiment.

FIGS. 2A-2C are cross-sectional side view of implants with different dimensions according to another embodiment.

FIG. 3 is a cross-sectional view of another embodiment of an implant.

FIGS. 4A-4B are perspective and cross-sectional side views, respectively, of another embodiment of an implant.

FIG. 5 is a perspective views of another embodiment of an implant.

FIGS. 6A-6B are perspective and cross-sectional views, respectively, of an implant according to one embodiment.

FIG. 7 is a perspective view of an implant according to another embodiment.

FIGS. 8A-8B are perspective and perspective cross-sectional views, respectively, of an implant according to another embodiment.

FIGS. 9A-9C are perspective, bottom, and cross-sectional (through Line A-A of FIG. 9B) views, respectively, of an implant according to still another embodiment.

FIG. 10 is a perspective view of yet another embodiment of an implant.

FIGS. 11A-11C are cross-sectional perspective, top, and perspective views of another embodiment of an implant.

FIG. 12 is a cross-sectional view of still another embodiment of an implant.

FIG. 13 is a cross-sectional view of another embodiment of an implant.

FIG. 14 is a cross-sectional, perspective view of an implant mold according to an embodiment.

FIG. 15 is a perspective view another embodiment of an implant mold.

FIGS. 16A-16C are perspective views of loading tools of a loading system.

FIGS. 16D-16E are partially cross-sectional side views of a loading tool of FIG. 16C according to one embodiment.

FIG. 16F is a perspective view of delivery device tools of a delivery system.

FIGS. 17A-17D are perspective views of an actuatable tool according to one embodiment.

FIG. 18 is a cross-sectional view of another embodiment of a loading tool.

FIGS. 19A-19B are side and front views, respectively, of conical rod and implant of FIG. 16A.

FIGS. 20-21 are side views of the implant being stretched over the conical rod.

FIG. 22 is a side view of the centering mandrel of FIG. 16A being inserted into the conical rod.

FIGS. 23-24 are side views of the implant compressor of FIG. 16A extending over and radially compressing the implant over the conical rod.

FIGS. 25A-25B are side and front views, respectively, of the implant compressor compressing the implant over the conical rod.

FIG. 26 is a side view of the conical rod being removed from within the implant.

FIGS. 27A-27B are side and perspective views of the centering mandrel being removed from within the implant and the implant compressor compressing the implant in a pleated configuration, respectively.

FIGS. 28-29A are side views of the finned mandrel of FIG. 16A being inserted into the implant.

FIG. 29B is a front view of FIG. 29A of the finned mandrel within the implant and the implant compressor.

FIG. 30 is a partially cross-sectional side views of the tapered tube of FIG. 16D being lubricated.

FIGS. 31-33 are partially transparent and cross-sectional side views of the implant being inserted into the tapered tube.

FIG. 34 is a partially cross-sectional side view of the implant compressor being removed from the implant and the tapered tube.

FIG. 35 is a partially cross-sectional, partially transparent, side view of the tapered tube being attached to the sheath holder of FIG. 16D.

FIGS. 36-40 are partially cross-sectional, partially transparent, side views of the tube of FIG. 16B being inserted into the implant with the rod of FIG. 16B, while the finned mandrel is being pushed out of and removed from the implant.

FIGS. 41A-41B are back and front views, respectively, of the implant within the tapered tube of FIG. 40.

FIG. 42 is a cross-sectional, side view of the centering mandrel being inserted through the implant.

FIGS. 43-46 are partially cross-sectional, side views of the implant being pushed into the sheath holder with the rod.

FIG. 47 is a partially cross-sectional, side view of the tapered tube and the sheath holder being detached.

FIGS. 48-50 are partially cross-sectional, side views of the implant being removed from the sheath holder and the centering mandrel being removed from the implant.

FIGS. 51-53 are partially cross-sectional, side views of the implant being reinserted into the sheath holder.

FIG. 54 is a partially cross-sectional, side view of the capsule loading section of FIG. 16D being attached to the sheath holder.

FIGS. 55-57 are partially cross-sectional, side views of the delivery device of FIG. 16F being inserted into the capsule loading section.

FIG. 58 is a partially cross-sectional, side view of an implant positioner within the delivery device extending partially out of the delivery device and into the capsule loading section while the loading grip of FIG. 16F is being attached to the delivery device.

FIGS. 59-63 are partially cross-sectional, side views of the implant being partially inserted into the implant positioner with the rod.

FIG. 64 is a partially cross-sectional, side view of the implant positioner and the implant retracting within the delivery device.

FIGS. 65-66 are partially cross-sectional, side views of the loading grip and capsule loading section, respectively, being removed from the delivery device.

FIGS. 67-70 are partially cross-sectional, side views of the implant being pushed into the delivery device with the rod and removing the centering mandrel.

FIG. 71 is a partially cross-sectional, end view of the implant within the delivery device.

FIGS. 72-74 are partially cross-sectional, side views of a first flange of the implant being deployed from the delivery device.

FIG. 75 is a partially cross-sectional, side view of the entire implant being deployed from the delivery device.

FIG. 76A is a perspective view of a delivery device according to one embodiment.

FIG. 76B is a partially transparent, exploded view of a handle of the delivery device of FIG. 76A.

FIGS. 76C-76D are perspective and side views of the balloon deflated and inflated, respectively, in front of the capsule of FIG. 76A.

FIG. 76E is a cross-sectional side view of the capsule of FIG. 76A.

FIG. 76F is a perspective view of the implant positioner of FIGS. 76C-76D.

FIG. 77 is a perspective, exploded view of a handle according to another embodiment.

FIGS. 78-79 are cross-sectional views of various embodiments of an insertion tube.

FIG. 80 is a perspective view of another embodiment of an actuation tube.

FIG. 81 depicts side views of various embodiments of a guidewire.

FIG. 82 is a perspective view of a capsule according to one embodiment.

FIG. 83 is a perspective view of a capsule according to another embodiment.

FIG. 84 is a perspective view of an implant positioner according to another embodiment.

FIGS. 85A-85B are perspective and cross-sectional views of an implant positioner according to still another embodiment.

FIGS. 86A-86E depict an implant being delivered and deployed within a body.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Referring generally to the disclosure, described herein are apparatus and methods to use a self-retaining implant or shunt to allow two organs, lumens, tissues, areas, and/or regions of a body to be connected, joined, or paired together. Further, described herein are devices and methods for loading the implant into a delivery device and deploying the implant within the body of a patient. The implant of the present disclosure may allow one body lumen to drain to another body lumen. Although the implant may be used with a variety of regions or structures within the body, the implant may be specifically designed to connect particular organs. For example, the implant of the present disclosure may join the gallbladder lumen and the duodenum lumen. This may, for example, allow the gallbladder to drain into the duodenum and allow gallbladder content to be removed from the body through the digestive system.

In order to load or fold the implant, loading devices may be used to fold and compress the implant into a compressed configuration in a deployment device. A deployment device may be used to deliver, implant, insert, or deploy the implant into a deployed configuration to a particular delivery site within the body. The methods and apparatus of delivery and deployment may provide a minimally invasive procedure and may allow the implant to be accurately delivered to the desired site within the patient and deployed in a controlled and predictable manner.

The apparatus and methods described herein may be used in conjunction with a variety of different medical or surgical devices and procedures. For example, the apparatus and methods described herein may be used with endoscopic ultrasound (EUS) and/or fluoroscopy in order to provide visualization of the devices and procedures. Optionally, the apparatus and methods described herein may or may not be paired with an echoscope or an endoscope. For example, the implant may optionally be delivered with a scope (such as an endoscope), such as through a working channel or over-the-wire of the endoscope. Further, the apparatus and methods described herein may be used for drug delivery to specific regions of the body.

The apparatus and methods described herein may move the treatment of a variety of conditions (such as biliary disease) from surgery to endoscopy, which may reduce amount of invasiveness of the procedures and interventions. Further, the cost of treatment, the patient recovery times and pain levels, and the morbidity and mortality rates may be reduced by using the apparatus and methods described herein. Additionally, with endoscopy, conscious sedation (managed by a sedation nurse) may be used instead of general anesthesia (managed by an anesthesiologist).

The apparatus and methods described herein may be used to address a variety of different medical needs and conditions, including but not limited to biliary disease, pancreatic pseudocyst, obesity, abscess drainage, and gastrointestinal (GI) obstructions.

The methods and apparatus described herein may be particularly beneficial for use for the treatment of biliary disease, including cholecystitis caused by gallstones. Over 1.2 million procedures are a result of biliary disease in the United States every year. These procedures primarily include cholecystectomy and endoscopic retrograde cholangiopancreatography (ERCP). Cholecystectomy is the surgical removal of the gallbladder and has over 700,000 procedures in the US every year. ERCP involves endoscopically accessing and clearing the bile duct and has over 500,000 procedures in the US every year.

Pancreatic pseudocysts, which have about 1,000 procedures in the US every year, are currently treated surgically, but a growing number of the pseudocysts are being addressed endoscopically. Obesity, which had about 210,000 procedures in the US in 2010, usually includes re-routing food in the GI tract to cause a reduction in the absorption of ingested calories and nutrients in order to cause weight loss. Abscess drainage may potentially be treated without external damage by draining the abscesses internally (from the site of the abscess to a nearby site in the GI tract). GI obstructions may potentially be treated by re-routing the flow in the GI tract to circumvent obstructions (e.g. strictures or tumors). These, as well as other medical conditions, may be treated with the apparatus and methods described herein.

Further, the methods and procedures associated with the apparatus described herein may be performed by a variety of different people. For example, surgeons, gastroenterologists, endoscopists, and/or members of the Society of American Gastrointestinal Endoscopic Surgeons (SAGES) may use or implement the methods and procedures, as well as use the apparatus.

Although the gallbladder and the duodenum are specifically addressed with respect to the apparatus and methods described herein, it is anticipated that the apparatus and methods may be used within a variety of different regions or structures within the body, as well as for a variety of different purposes, procedures, or diseases. For example, the apparatus and methods described herein may be used to drain adjacent organs or cysts, such as gastric pseudocysts, or cysts that may result due to pancreas leakage. The apparatus and methods described herein may also be used for bariatric surgeries or procedures, such as reducing the passage or bypassing at least one section of the small intestine by attaching different regions of the small intestine with the implant.

Further, the implant, delivery devices, and loading devices (and the corresponding methods) may be used in conjunction with each other, separately from each other, and/or with additional devices.

Referring generally to the disclosure and according to one embodiment, an implant 10 may be used to connect at least two areas or organs of the body of a patient. The implant 10 may optionally allow material to move between two organs through a central lumen or aperture within the implant 10. The implant 10 may have opposing flanges to hold or secure the tissue walls of the organs together therebetween in order to connect two organs. The implant 10 may be configured to be a permanent or temporary fixture within the body. For example, due to pressure from the opposing flanges, the implant 10 may cause the secured tissue between the flanges to necrose or die. The necrosed or dead tissue may subsequently break off from the surrounding viable tissue. Since the implant 10 is attached to the tissue, the implant 10 may also break off with the dead tissue. Depending on the positioning of the implant 10, the implant 10 may be excreted through the digestive system of the patient.

In order to deliver the implant 10, the implant 10 may be folded with folding devices into a compressed configuration in order to fit within and be loaded into a delivery device 100 to be properly deployed within the body of the patient. For example, a central portion of the implant 10 may be expanded or stretched to move the two flanges away from each other. The implant 10 may be subsequently compressed along the longitudinal axis of the implant 10. While the implant 10 is in the compressed configuration, the implant 10 may be advanced into a capsule in the end of the delivery device 100.

Once the implant 10 has been loaded into the delivery device 100, the implant 10 may be delivered to the desired location within the body of the patient and deployed. The capsule (containing the compressed implant 10) of the delivery device 10 may be advanced into the body (through, for example, the mouth of the patient and into the esophagus and the stomach) toward the deployment site of the first cavity (e.g. the duodenum). The capsule may be advanced further into a second cavity (e.g. the gallbladder). In order to deploy the implant 10, a central lumen of the delivery device 100 may push the implant 10 partially out of the capsule such that one of the flanges is deployed within the gallbladder. The capsule may then be retracted back into the duodenum and the other flange may be deployed by pushing the implant 10 completely out of the capsule (and, thus, out of the delivery device). Accordingly, the two flanges may each be located in a different cavity or organ and the implant 10 may secure the two cavities together. The delivery device may be subsequently removed from the body.

### Transluminal Implant

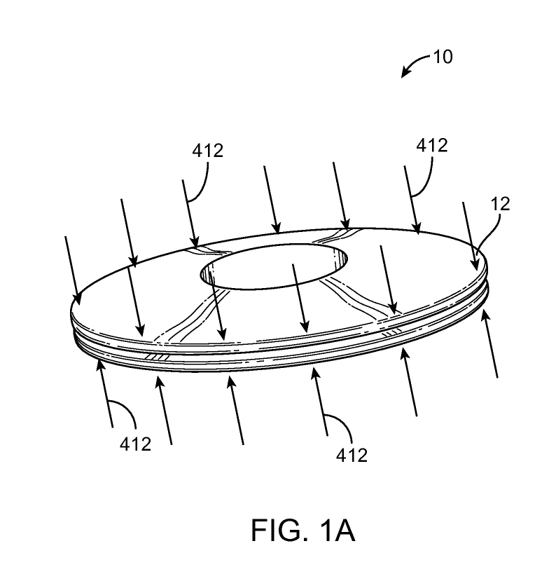

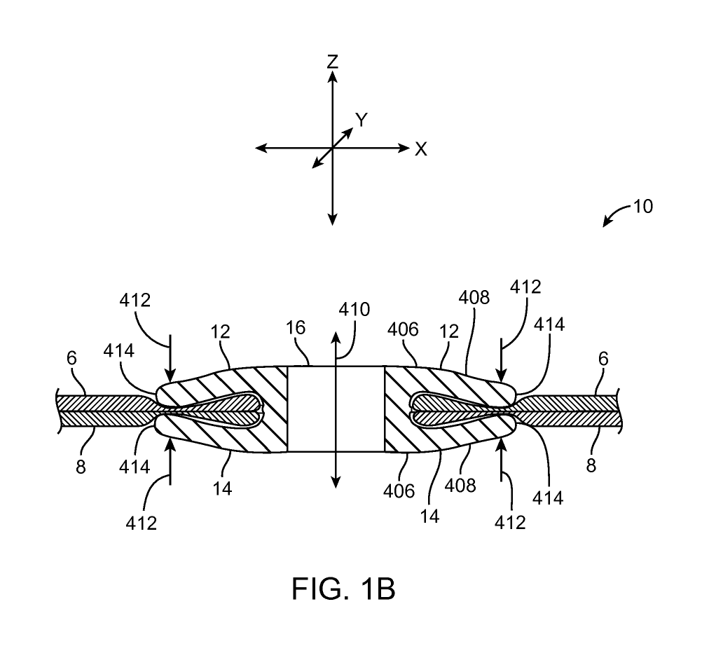

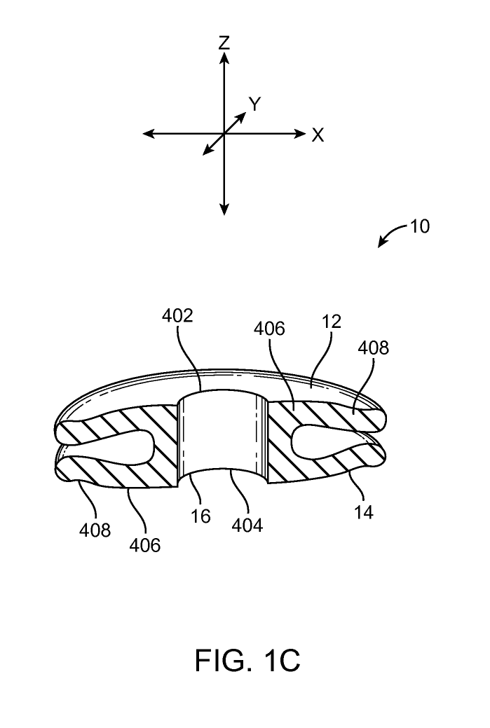

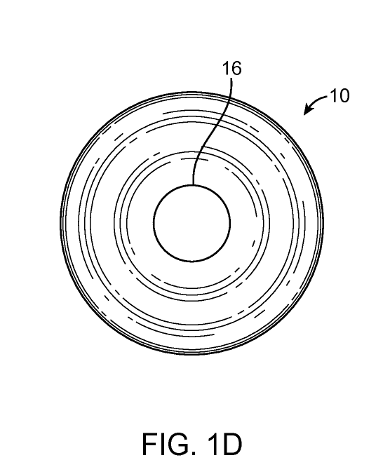

According to various aspects of the present disclosure and as shown in FIGS. 1-13, a transluminal device, medical apparatus, device, shunt, or implant 10 (or implant 300, 310, 320, 330, 340, 350, 360, 370, 380, 390, 500, 510, 520 or 530) for implantation in a body is provided and may be used to connect or attach different cavities, areas, or organs within the body and/or form a shunt between two tissues, resulting in immediate treatment of the underlying condition and relief of symptoms. The implant 10 may include a central passageway, aperture, tube, or lumen 16 (or lumen 306, 316, 326, 336, 346, 356, 366, 376, 386, 396, 506, 516, 526 or 536) along a central or longitudinal axis to allow material to move in at least one direction through the implant 10. Therefore, the implant 10, as well as the resulting connection or junction between the organs, may be a permanent or temporary fixture or implant within the body, depending on the desired use. The lumen 16 may be formed by apertures on opposing flanges.

The implant 10 may further include opposing anchoring flanges 12 and 14 (or flanges 302, 312, 322, 332, 342, 352, 362, 372, 382, 392, 502, 512, 522 or 532 and 304, 314, 324, 334, 344, 354, 364, 374, 384, 394, 504, 514, 524 or 534) extending on either end of the lumen 16 to exert a compressive force on and retain tissue between the flanges 12 and 14. The flanges 12 and 14 may be disc-shaped with a central first and second aperture, respectively. The ends of the lumen 16 may attach or fit to the first and second apertures to form a “reel-shaped” object capable of receiving tissue between the flanges 12 and 14. The applied pressure or compressive force secures or holds the implant 10 to at least one tissue and in place within the body. Further, the compressive force firmly holds, approximates, or apposes the walls of at least two body lumens or cavities together to maintain and seal the connection or junction between the two cavities.

A connection or fistula may be formed or created between the two cavities or organs within the body and/or around the implant as a result of the pressure necrosis. While implanted, the implant and the collected fluids may be completely internal within the body of the patient. Once the fistula is mature, the implant 10 may no longer be required to remain in place within the body in order to maintain the connection between the bodily lumens. Accordingly, the compressive force of the implant 10 may cause the tissue to necrose and may break off (with the necrotic tissue) from the surrounding tissue. The implant 10 (and any attached necrosed tissue) may be self-remove, separate, or detach from the surrounding tissue (as described further herein) and pass out of the body (through, for example, the intestinal tract), eliminating the need for further follow-up or post-implantation endoscopic procedures or surgeries to maintain or remove the implant 10. This may further eliminate the need to surgically create a connection between two cavities.

Referring now to FIGS. 1A-1D, there is shown the implant 10 according to one embodiment. The implant 10 may include a body defining a central aperture, tube, hole, hub, opening, passageway, or lumen 16, which may be used to connect the first lip, washer, disk, tissue engager, or flange 12 and the second lip, washer, disk, tissue engager, or flange 14. The lumen 16 may extend along (and define) the central axis (e.g. the z-axis) of the implant 10 from a first end 402 to a second end 404, as shown in FIG. 1B.

Although the lumen 16 is shown in FIGS. 1A-1D as round or circular, it is anticipated that the lumen 16 may be any shape, including but not limited to oval, tubular, rectangular, pentagonal, and hexagonal. The dimensions of the inner diameter of the lumen 16 may depend on the size or volume of the intended material to pass or flow through the lumen 16, as well as any tools the lumen 16 may accommodate. For example, the inner diameter of the lumen 16 may be approximately 5-10 mm. The thickness, height or length (e.g. the distance between the first end 402 and the second end 404 along the central axis) of the lumen 16 may vary according to the desired configuration, such as the intended number of tissues the implant 10 will secure and the thickness of the tissues. Since the length or thickness of the lumen 16 may directly affect the relative positioning and spacing of the flanges 12 and 14, the lumen length may also affect how tightly the tissues may be secured or held by the implant 10 (which may also depend on the configuration and dimensions of the flanges 12 and 14). According to one embodiment, the length of the lumen 16 may at least span the thickness of the intended tissue(s) to be secured within the implant 10.

According to one embodiment of the present invention as shown in FIG. 1B, the lumen 16 may provide or create a passageway 410 to allow material (such a solids, gas, fluids, and/or liquids) to flow, move, or pass between the two cavities in at least one direction. For example, if the implant 10 attaches the gallbladder and the duodenum, the gallstones may move through the lumen 16 from the gallbladder to the duodenum. However, it is anticipated that the lumen 16 may be closed off and may prevent the movement of material between the cavities while maintaining the connection between the cavities with the first and second flanges 12 and 14. Alternatively or additionally, the lumen 16 may also be used as a working channel within the body to allow tools, such as endoscopes, to move into or access a particular area of the body.

The first flange 12 and the second flange 14 may extend radially from at least a portion of the circumference of the lumen 16 along the first and second ends 402 and 404, respectively, of the lumen 16 such that the flanges 12 and 14 at least partially surround the lumen 16. According to one embodiment, the first and second flanges 12 and 14 may extend approximately along the x- and y-axes from the lumen 16, such that base portions 406 of the flanges 12 and 14 (which may be attached to the lumen 16) are substantially, about, or generally perpendicular to the central axis of the lumen 16. For example, at least one of the base portions 406 may be approximately 90° to the central axis, plus or minus 30° (e.g. at least one of the base portions 406 may be positioned approximately between 60-120° to the central axis). Therefore, there may be a variance of up to 60° that at least one of the flanges 12 or 14 may have in relation to the central axis. The angle of variation from true perpendicular (e.g. 90°) may be the same or different for the flanges 12 and 14. According to one embodiment, at least a portion of the base portions 406 of the flanges 12 and 14 may be about parallel.

As shown in FIGS. 1B-1C, the base portions 406 (or base portions 337) of the first and second flanges 12 and 14 may extend radially away from the lumen 16 into angled portions 408 (or angled portions 338), which may be at a variety of different angles from the base portions relative to the central axis of the lumen 16, according to the desired configuration. In the embodiments where at least one of the angled portions 408 are at least partially angled toward each other, as shown in FIG. 1B, the angled portions 408 may clamp, receive, compress, couple, and/or hold structure(s) or tissue(s) between the angled portions 408 of the flanges 12 and 14 to help retain the position of the implant 10 and/or to cause necrosis in the tissue, as described further herein. Therefore, the distance between the angled portions 408 may be less than the distance between the base portions 406. Accordingly, a small distance between the angled portions of the first and second flanges 12 and 14 may result in more tissue necrosis due to lack of circulation within the tissues and/or hold the position of the implant 10.

According to one embodiment, the angled portions 408 may be angled toward each other at an angle between approximately 1 and 180°. When the angle is approximately 180°, the angled portions 408 may be about parallel to the base portions 406.

According to another embodiment, at least one of the angled portions 408 may be stepped down or slightly offset from the base portion 406 (instead of a smooth or continuous surface between the angled portion 408 and the base portion 406), allowing the angled portion 408 to be physically closer to the opposing flange. Further, the angled portion 408 may be angled or approximately parallel to the base portion 406.

According to another embodiment, at least one of the flanges 12 or 14 may have a second angled portion that may be at a different angle from the angled portion 408 or the base portion 406. The second angled portion may be angled toward or away from the opposing flange. Alternatively or additionally, the second angled portion may have a step down region, configuring the second angled portion to be physically closer to the opposing flange. It is also anticipated that at least one of the angled portion 408 or the second angled portion may have a step up region.

According to one embodiment, the implant 10, which may optionally have the angled portions 408, may still be able to rotate or spin over the tissue while maintaining the implant's functionality and operation as a shunt between two body lumens or organs and without moving out of an effective position and

As shown in FIG. 1B, the first and second flanges 12 and 14 may be configured to secure at least two tissues, such as a duodenum tissue 6 and a gallbladder tissue 8, therebetween. The first flange 12 may be configured to be deployed and disposed in a first or proximal cavity, such as the duodenum. The second flange 14 may be configured to be deployed and disposed in a second or distal cavity, such as the gallbladder. Therefore, the first flange 12 may be positioned proximal to the duodenum inner wall or tissue 6, while the second flange 14 may be positioned proximal to the gallbladder inner wall or tissue 8. Therefore, the lumen 16 may extend through the two tissues and connect the first and second cavities.

The implant 10 may configured and sized to fit at least one tissue in the patient between the flanges 12 and 14 (and therefore may connect at least two body lumens or cavities). For example, the duodenum tissue wall and the gallbladder tissue wall may be clamped and compressed between the first and second flanges 12 and 14 to attach the duodenum and the gallbladder with the implant 10. However, it is anticipated that the flanges 12 and 14 may secure any number and type of tissue therebetween, connecting any number of bodily cavities. For example, the implant 10 may be configured to hold 6 or 7 tissues between the flanges 12 and 14. The compressive force between the flanges 12 and 14 (due to the configuration and described further herein) may allow the flanges 12 and 14 to secure, hold, and/or necrose the tissue therebetween and prevent the tissue walls from unintentionally separating.

Due to the geometry and materials of the implant 10, the flanges 12 and 14 may apply a spring force to the tissues therebetween. The pressure on the tissue from the flanges 12 and 14 may prevent implant 10 migration or displacement, prevent leakage from occurring out of the connected bodily lumens (and into, for example, the peritoneum), facilitate the formation of a fistula around the implant 10, and cause pressure necrosis to occur in the clamped tissue. Opposing, applied, exerted compressive pressures or forces 412 of the first and second flanges 12 and 14 (and, therefore, the configuration of the implant 10) may allow the implant 10 to pinch, clamp, secure, hold, or compress at least one tissue therebetween. Accordingly, multiple tissues (and body lumens) may be held together.

The force of the flanges 12 and 14 on the tissue may allow the flanges 12 and 14 to act as an anchoring mechanism for the implant 10 within the body and prevent any unintentional migration. The implant 10, therefore, is self-retaining and self-anchoring within the body.

Depending on the desired use and application (e.g. connecting the duodenum to the gallbladder), the amount of applied pressure may vary. For example, the amount of necrosis the tissue between the flanges 12 and 14 may undergo may depend on the amount of pressure exerted on the tissue from the flanges 12 and 14. Certain characteristics and dimensions of the implant 10 may affect the amount of applied pressure after deployment, including but not limited to the flange configuration, the flange dimensions (e.g. flange diameters and thickness), the flange material, and the material properties (e.g. material hardness). Further, in addition to the implant configuration, the tissue type and thickness may affect the amount of applied pressure or force 412.

Depending on the amount of applied pressure, at least the edges of the surrounding intact tissue may anneal, scar, attach, or adhere together as a result of the pressure from the flanges 12 and 14. Further, the initial puncture and dilation of the tissue, as well as the introduction of the implant 10 may cause scar tissue to form around the implant 10. According to the configuration of the implant 10, scar tissue may form around or surround at least a portion of the implant 10 and a fistula may form (due to the pressure necrosis), which may further minimize leakage around the implant 10 or after the implant 10 has detached from the surrounding tissue. According to one embodiment, a relatively larger fistula may be formed from the pressure necrosis around the periphery of the implant 10. The larger fistula may be less likely to clog or close over time and may allow for the passage of larger tools or bodily debris (e.g. gallstones) into or out of the body cavities.

If the flanges 12 and 14 are configured to exert sufficient pressure to cause necrosis around the surrounding tissue (depending on the amount and configuration of applied pressure), the implant 10, with the necrosed tissue (such as a circular region of necrosis, which may result from the implant 10 in FIG. 1A), may eventually completely detach or break off from the surrounding viable tissue and lumen walls and naturally pass out of the body. The detachment may further result in a necrosis aperture or connection between the lumens that may remain without the implant 10. Depending on the region of the body the implant 10 is released into (such as the duodenum), the implant 10 may be expelled from the body through the digestive system of the patient (e.g. by peristalsis in the intestine). Alternatively or additionally, the implant 10 may be made out of materials such that the implant 10 is reabsorbable and/or dissolvable within the body after a certain period of time, eliminating the need to pass through the body to be eliminated. These features of the implant 10 may obviate the need to perform additional procedures to manipulate, maintain, or remove the implant 10. Alternative to a temporary implant, however, the implant 10 may be designed to be a permanent fixture within the body and may not detach from the surrounding tissue.

The configuration and shape of the implant 10 (e.g. the flanges 12 and 14) may affect the amount of applied compressive pressure or force 412 the flanges 12 and 14 exert on each other and the tissue therebetween. For example, with sufficient pressure, the opposing forces of the first and second flanges 12 and 14 may induce pressure necrosis in at least a portion or region of the tissue between the flanges 12 and 14. The continuous compressive pressure or force exerted on the tissue by the flanges 12 and 14 be strong enough to exclude, prevent, or reduce blood circulation from flowing within a portion of retained tissue, which may cause at least a portion of the tissue to die or undergo necrosis. According to one embodiment, the forces of the flanges 12 and 14 on the tissue may create a fluid-tight seal. However, the applied pressure may not excessive to prevent unintentional injury. Depending on the degree and configuration of necrosis, the necrosed tissue (with the attached implant 10) may detach from the surrounding viable tissue. For example, if the necrosis occurs within a continuous region of tissue (e.g. a circle), the necrosed tissue with the implant 10 may detach from the surrounding tissue.

The configuration of the angled portions 408 may affect the amount of compressive force 412 applied to the tissue along an outer perimeter of the flanges 12 and 14. As shown in FIGS. 1B-1C, the first and second angled portions 408 may be curved or angled inward towards each other, creating a smaller gap between the first and second angled portions 408 than the first and second base portions (which may be approximately perpendicular to the lumen 16 and therefore approximately parallel to each other). The distance (along the z-axis) between the closest portions of the flanges 12 and 14 (e.g. the ends) may affect the amount of necrose and change the pressure-necrosis performance. The more that the ends or angled portions 408 of the flanges 12 and 14 are angled towards each other or closer, the more compressive force 412 is exerted on the tissue, resulting in a stronger hold and potentially more necrosis within the tissue.

Depending on the degree of curvature or the angle, at least a portion of the flanges 12 and 14 (such as the outer edge or ends of the first and second angled portions 408) may touch or have at least one point of contact in an expanded or deployed configuration 220. According to one embodiment, the angled portions 408 may contact each other around the entire circumference of the implant 10 in the deployed configuration 220 (with no tissue therebetween). However, according to another embodiment, there may be a small gap or space between the flanges 12 and 14 in the deployed configuration 220.

The location and pattern of applied force 412 may affect how the tissue may necrose FIG. 1A depicts a continuous round region of pressure or force 412 that may be applied by the flanges 12 and 14 onto the tissue. The configuration and flange design and patterns may cause the tissue to necrose in a particular manner or geometric shape. For example, the force 412 shown in FIG. 1A is applied in continuous ring, which may result in a circular region or disc of necrosed tissue. Alternatively, the applied force 412 may be discontinuous, thereby allowing sufficient circulation to keep the tissue alive. This may allow the flanges 12 and 14 to cause only sections of the tissue to necrose (for example, to create flaps of necrosed tissue, as described further herein) or to simply hold the tissue therebetween.

Ends, rims, outer perimeters, or edges 414 of the flanges 12 and 14 may include a lip or bump along the outermost perimeters of the flanges 12 and 14 to further compress or pinch tissue between the flanges 12 and 14. The edge 414 may be thicker than the inside portions of the flanges 12 and 14 in order to provide more strength to grip and pinch tissue therebetween.

Further, the tissue-contacting surfaces, such as the ends of the angled portions of the flanges 12 and 14, the edge 414, or the outer surface of the lumen 16, the flanges 12 and 14, may have atraumatic edges to prevent any unintentional trauma to the surrounding tissue or organs. For example, the tissue-contacting surface may have smooth or gently rounded or radiused edges 414 and may not incorporate sharp edges or points, as shown in FIG. 1B.

The exerted or applied force 412 may be calculated over a circumferential dimension (e.g., a circumferential millimeter or inch). According to one embodiment, the flanges 12 and 14 may require about 50 grams of force to pull apart the flanges 12 and 14 when the flanges 12 and 14 are separated by 2 mm (the 2 mm may represent the tissue held between the flanges 12 and 14). Depending on the desired force and size of implant, a smaller implant 10 may be used with a higher amount of force. For example, the lumen 16 diameter may decrease from a 10 mm hole to a 5 or 6 mm hole, and the flanges 12 and 14 may be configured to maintain sufficient pressure to cause necrosis.

The implant 10 may maintain a constant and/or consistent compressive clamping force or pressure overtime to the tissue walls between the flanges 12 and 14 to prevent the implant 10 from falling out or moving prematurely. For example, the gripping pressure between the flanges 12 and 14 may remain consistent throughout the life of the implant 10 inside the patient. Accordingly, the active approximation of the tissue walls may prevent migration of the implant 10 and create a tight seal around the implant 10 to minimize or prevent leaks. However, it is anticipated that the implant 10 may be designed to fatigue at a particular rate to ensure that the implant 10 will fall out after the desired amount of time.

The flanges may include additional features to provide certain patterns or amounts of necrosis. The additional features, such as textured portions, may disturb, block, or interrupt the blood flow through a particular section of tissue. Alternatively, the additional features may allow blood flow within a particular section of tissue. The additional features may apply a continuous line, pattern, or region of applied pressure to the tissue to allow the necrosed tissue to break off from the surrounding tissue. Alternatively, the additional features may apply a discontinuous line, pattern, or region of applied pressure to allow the necrosed tissue to remain attached to the surrounding tissue. For example, the implant may include features such as longitudinal or radial grooves 428, fins, bumps, lines, or ribs along the inside of at least one of the flanges 12 and 14 to contact the tissue in a particular manner and potentially cause the tissue to necrose, perforate, and/or scar in a variety of different designs, shapes, and geometric patterns, which may facilitate subsequent procedures and/or enhance tissue fixation and the fistula structure.

The ribs may protrude into the tissue to increase the amount of necrosis or to cause necrosis lines (or a pattern) instead of (or in addition to) a necrosis aperture. For example, the implant 10 may apply a thin line of pressure to create at least one slit, slot, or necrotic line, which may intersect at a middle region (similar to the shape of an asterisk) and creating flaps of necrosed tissue still attached to the surrounding tissue by a section of healthy tissue. Once the implant 10 detaches from the surrounding tissue, the slit or necrotic lines may function as a tissue valve that may open and close depending on the pressure differential between the two lumens. For example, with enough pressure from the gallbladder, the tissue valve may open and allow material (such as gallstones) to move into the duodenum. Viable tissue may surround the tissue valve.

The additional features or the geometry of the flanges 12 and 14 may further be beneficial in the event that there is a weakness or leak along the perimeters of the flanges 12 and 14, which may cause at least a portion of the tissue between the flanges 12 and 14 is supplied with blood. Without additional features to induce necrosis and with the leak, the tissue may not completely necrose and may not allow the implant 10 to completely break off from the viable tissue. The additional features may ensure that necrosis occurs, even in the event of a leak or weakness between the flanges 12 and 14.

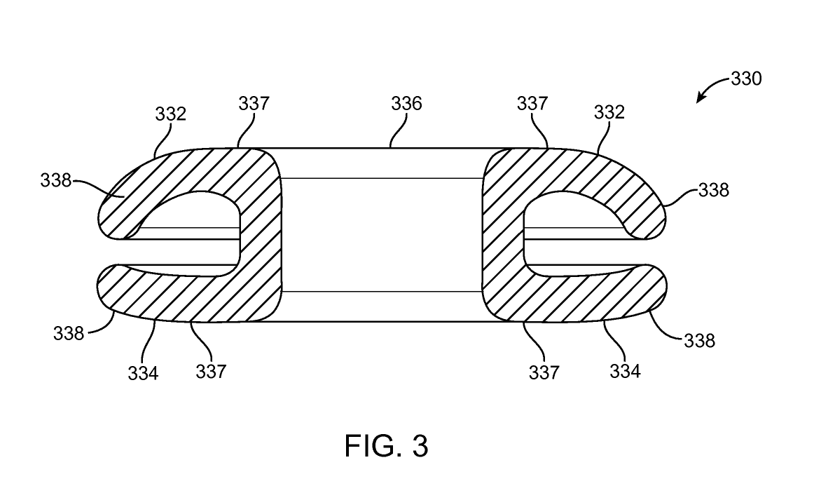

According to another embodiment as shown in FIG. 3, there is shown an implant 330 with flanges 332 and 334 and lumen 336. At least one of the flanges 332 and 334 may have a base portion 337 and an angled portion 338. At least one of the angled portions 338 of at least one of the flanges 332 or 334 may have an exaggerated inward curve or angle toward the other flange to increase the pinch force, and therefore also to accentuate the necrosis pressure on the tissue between the flanges 332 and 334. Accordingly, the implant 330 may be asymmetric along a midline of the implant parallel to the x-y plane, as shown in FIG. 3. However, it is anticipated that the flanges 332 and 334 may both be curved in toward each other such that the implant 330 is symmetric. Further, the flanges 332 and 334 may have the same diameter (or the outer diameter of one of the flanges 332 or 334 may be greater than that of the other flange, as described further herein).

The material, along with the configuration and the tissue properties, of the implant 10 may also affect the amount of applied pressure. If the flanges 12 and 14 are more flexible (due to the material properties and/or the flange dimensions) and/or the tissue is relatively thin, less pressure may be exerted and less necrosis may result. Further, the material texture of the implant 10 may vary depending on the desired amount of necrosis. For example, a relatively rougher texture may increase, promote, or speed-up necrosis.

However, according to another embodiment, the implant 10 may not induce necrosis to prevent the implant 10 from falling out. Accordingly, the flanges 12 and 14 may provide sufficient force to secure the implant 10 to the surrounding tissue, but the flanges and 14 may be relatively parallel instead of pinching or angling inward towards each other. Scar tissue may still form, but may be insufficient to cause necrosis. This may be beneficial if a valve is integrated with the lumen 16 of the implant 10 or in the case that a temporary connection between two cavities is desired.

For example, gallstones may be more prevalent during pregnancy. Accordingly, the implant 10 may provide a connection or aperture between the gallbladder and the duodenum during pregnancy. After pregnancy when no connection is needed, the implant 10 may stay within the body and the aperture within the lumen 16 may be sealed with a plug to allow the gallbladder may regain its function.

The implant 10 may be sized according to the desired configuration, use and application. For example, the implant 10 may be delivered through, over, or next to a working channel of a delivery device (such as an endoscope or catheter) and through at least one body lumen. Therefore, the implant 10 may be minimally sized in order to fit properly with the delivery device in a compressed configuration 210. However, the size of the implant 10 may also affect how the implant 10 interacts with the tissue. For example, the size may affect how large the aperture is within the tissue or how tightly the implant 10 may hold the tissue between the flanges 12 and 14.

The overall size and configuration of the implant 10 may also allow the implant 10 to pass into one of the newly-joined body lumens once pressure necrosis has occurred, a mature fistula has formed, and the implant 10 (potentially with a section of necrosed tissue) has detached from the surrounding tissue. Therefore, the shape, size, and composition of the implant 10 may allow the implant 10 to pass out of the body naturally (e.g. peristalsis in the intestinal tract) and/or be retrievable in a follow-up medical procedure (such as an endoscopic procedure). Alternatively or additionally, the implant materials may be resorbable or dissolvable over a period of time in the body, as described further herein.

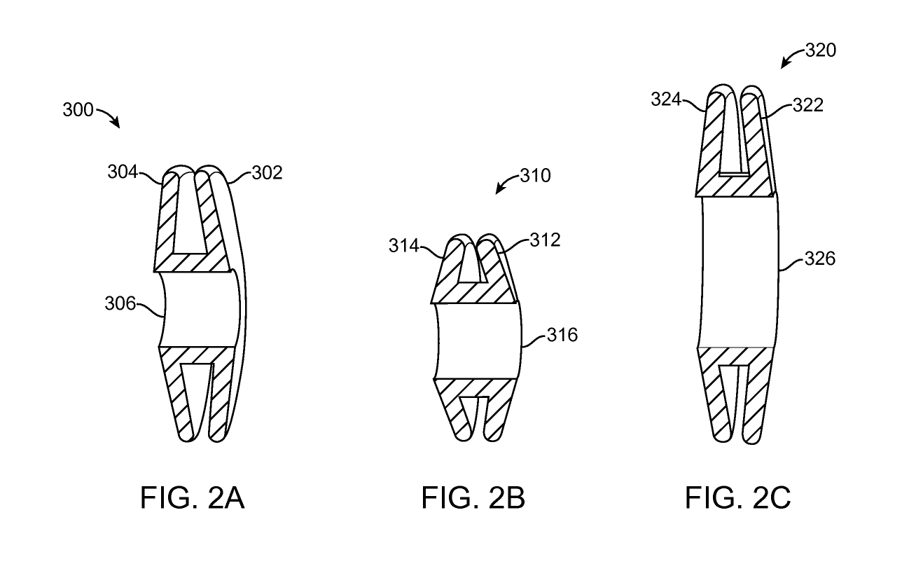

Therefore, the overall size, volume, and mass of the implant 10 and the size of each of the components within the implant 10 may vary. FIGS. 2A-2C, for example, shows three implants 300, 310, and 320 with different sized flanges and different sized lumens.

The actual dimensions may vary according to the desired configuration. However, according to some embodiments, the dimensions of the implant 10 may be within a range. For example, the outer diameter (along the x- and y-axes) and the height or thickness (along the z-axis) of the implant 10 may vary. According to one embodiment, the outer diameter of the implant 10 (and the outer diameter of the flanges 12 and 14) may range from 7 to 35 mm. According to another embodiment, the outer diameter may range from 10 to 30 mm. According to yet another embodiment, the outer diameter may range from 11.4 to 29 mm. According to still another embodiment, the outer diameter may be approximately 18.3 mm. The location where the flanges 12 and 14 are closest together along the z-axis (and therefore the region where the most pressure is applied to the tissue from the flanges 12 and 14) may be referred to as the pinch diameter. The pinch diameter of the implant 10 may range between 15 to 25 mm. According to another embodiment, the pinch diameter may range between 17 and 22 mm.

The height or thickness of the implant 10 (which may correspond to the height or thickness of the lumen 16) along the z-axis may range from 1.2 to 7.6 mm. According to another embodiment, the implant thickness may range from 1.7 to 6.4 mm. According to yet another embodiment, the implant thickness may range from 2.36 to 5.87 mm.

The inner diameter of the flanges 12 and 14 (which may correspond to the inner diameter of the lumen 16) may range from 2 to 15 mm. According to another embodiment, the inner diameter may range from 5 to 10 mm.

The thickness of material of the implant and of the individual the flanges 12 and 14 may range from 0.25 to 1.65 mm. According to another embodiment, the flange thickness may range from 0.5 to 1.3 mm. According to yet another embodiment, the flange thickness may range from 0.6 to 1.35 mm. According to still another embodiment, the flanges may be approximately 1.346 mm thick. The flanges 12 and 14 may have the same thickness or may have different thicknesses, according to the desired configuration. Additionally, the thickness of the flanges 12 and 14 may radially vary (such that, for example, the flange is thicker or thinner along an inside portion of the flange compared to an outside portion of the flange).

The distance or gap between the ends of the flanges 12 and 14 in the deployed configuration 220 (with no tissue positioned therebetween) may range from 0.25 to 1.77 mm. According to another embodiment, the distance may range from 0.38 to 1.4 mm. According to yet another embodiment, the distance may range from 0.5 to 1.35 mm. The radius of the flange edges 414 may range from 0.38 to 0.89 mm. According to another embodiment, the radius may range from 0.5 to 0.69 mm.

The implant 10 (such as the first and second flanges 12 and 14) may be shaped according to the desired configuration, use, and application. According to one embodiment as shown in FIGS. 1A-1D, the implant 10 may be symmetrical through different planes, such as through the central axis. For example, the implant 10 may be symmetrical through the x-y plane through a middle section of the lumen 16 and/or the implant 10 may be symmetrical through the x-z plane or y-z plane through a central axis of the lumen 16.

More specifically, the flanges 12 and 14 may be shaped and sized generally the same or be symmetrical mirror images of each other (as shown in FIGS. 1A-1D). According to another embodiment, the flanges 12 and 14 may be offset mirror images of each other. The flanges 12 and 14 may have the same internal and/or external geometry (including, but not limited to, the inner diameter and the outer diameter). For example, as shown in FIGS. 1A-1D, the flanges 12 and 14 may be disk shaped or generally round or circular around the lumen 16 and the diameters of the flanges 12 and 14 may be approximately equal.

Referring now to FIGS. 2A-2C, there is shown three implants 300, 310, 320 with flanges 302 and 304, 312 and 314, and 322 and 324 and lumens 306, 316, and 326, respectively. The amount that the first and second flanges are angled toward each other may change depending on how securely the tissues may be secured or pinched therebetween, as well as the other dimensions within the implant. For example, as shown in FIG. 2A-2C, the angle of the flanges may depend on the height or thickness of the lumen and the length of the flanges. As shown in the implant 300 in FIG. 2A, if the flanges 302 and 304 are longer, the flanges 302 and 304 may have a relatively larger acute angle between the flanges 302 and 304 and the lumen 306. As shown in the implant 310 in FIG. 2B, if the flanges 312 and 314 are shorter, the flanges 312 and 314 may have a relatively smaller acute angle between the flanges 312 and 314 and the lumen 316. As shown in the implant 320 in FIG. 2C, the lumen 326 may have a relatively larger diameter, with short or long flanges 322 and 324 (depending on the desired flange length, lumen size, and implant size). As further shown in FIG. 2A-2C, the flanges may optionally be relatively straight along the length of the flanges.

According to alternative embodiments as shown in FIGS. 3-13, the implant may be asymmetrical through at least one plane and the flanges may have different shapes and/or sizes, to optimize performance and according to the desired use and configuration. For example, the flanges may be shaped and sized such that the implant may preferentially or differentially fall out on, move toward, or be biased toward (intentionally or unintentionally) the side of the first flange 12 into, for example, the duodenum, instead of the second flange 14. For example, the first flange 12 may be more flexible due to geometry and/or material. The implant 10 may subsequently be passed out of the body through the digestive system. Therefore, the implant 10 may be positioned such that the first flange 12 is downstream from the second flange 14. “Downstream” may be closer, for example, to being expelled out of the body through the digestive system (e.g. the duodenum may be considered “downstream” from the gallbladder).

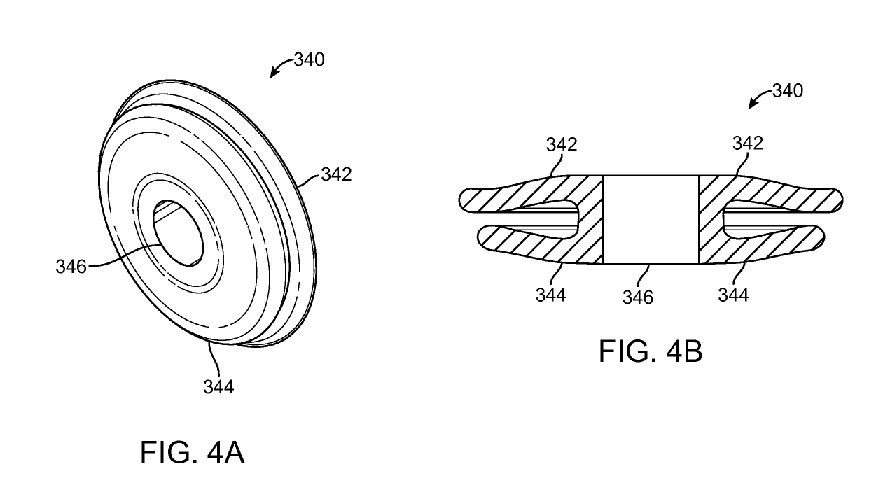

For example, as shown in FIGS. 4A-4B, an implant 340 may have a first flange 342 (which may be located in the duodenum), which may have a larger outer diameter than a second flange 344 (which may be located in the gallbladder) in order to preferentially expel or eject the implant 340 into the duodenum, rather than the gallbladder. According to one embodiment as shown in FIGS. 4A-4B, the second flange 344 may have a diameter of approximately 18 mm and the first flange 342 may have a diameter of approximately 20 mm, which results in an approximately 1 mm radial difference in flange diameter between the flanges 342 and 344. The radial difference may bias the implant 330 to preferentially fall out or move toward the side with the larger flange (e.g. first flange 342) into, for example, the duodenum. The larger first flange 342 may provide more resistance and make it more difficult for the implant 330 to move or expel toward the second flange 344, instead of the first flange 342. The implant 330 may detach from the surrounding tissue, fall out, be expelled, or be ejected due to a variety of reasons, including tissue necrosis as explained further herein. Although the flanges 342 and 344 may have different sizes, the flanges 342 and 344 may optionally have generally same shape. It is anticipated, however, that the flanges 342 and 344 may have different shapes according to the desired configuration. It is further anticipated that the flange 344 may have a larger diameter than the flange 342. The implant 340 may include a lumen 346.

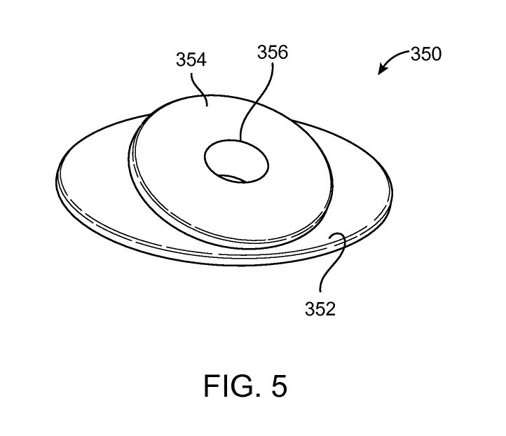

According to another embodiment as shown in FIG. 5, an implant 350 may have flanges 352 and 354 and a lumen 356. The flange 352 may have an oval shape, which may extend beyond the outer diameter of the flange 354 along at least the major axis of the flange 352. The oval shape of the flange 352 may bias the implant 350 to move toward the flange 352 within the body (thereby preventing the implant 350 from moving toward the flange 354). According to one embodiment, the dimensions of the major and minor axes of the flange 352 may be approximately 27 mm and 18.3 mm, respectively. As shown in FIG. 5, the flange 354 may have a circular shape. However, it is anticipated that the flange 354 may also have any shape according the desired configuration, including oval.

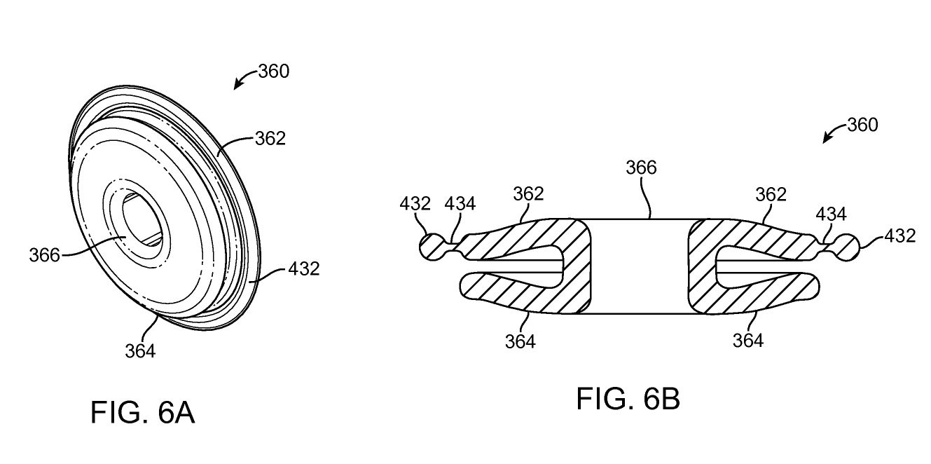

According to another embodiment and as shown in FIGS. 6A-6B, an implant 360 may have flanges 362 and 364 and a lumen 366. The flange 362 may have an extension along at least a portion of the perimeter to bias the implant 360 to fall out toward the flange 362. For example, the edge of the flange 362 may have an exaggerated flange or an additional outer ring, bump, or lip 432. The outer lip 432 may be attached to the outermost perimeter of the flange 362 with a flange web or extension 434. The flange extension 434 may a relatively thinner or more flexible section of material. The outer lip 432 may allow the outer diameter of the flange 362 to be increased in order to bias the implant 360 to fall out toward the flange 362 without significantly increasing the bulk of the flange 362. For example, the outer lip 432 and the flange extension 434 may increase the outer diameter of the flange 362 from 18 mm to 25 mm (however, it is anticipated that the outer lip 432 may increase the outer diameter by any amount). Further, the outer lip 432 may further be configured to be a rigid structure that will remain intact and relatively planar in order to resist collapsing inward. A stiff outer lip 432 of the flange 362 may further prevent the implant 360 from moving through the aperture or hole in the tissue, toward the flange 364. However, due to the flexibility of the flange extension 434, the outer lip 432 may be moved, bent out of the way, or gripped if needed. According to one embodiment, the radius of the lip 432 may range from 0.02 to 0.03 mm and the thickness of the extension 434 may range from 0.01 to 0.02 mm. According to another embodiment, the radius of the lip 432 may be approximately 0.025 to 0.027 mm and the thickness of the extension 434 may be 0.015 mm. Alternatively or additionally, the flange 364 may have the lip 432 and the extension 434.

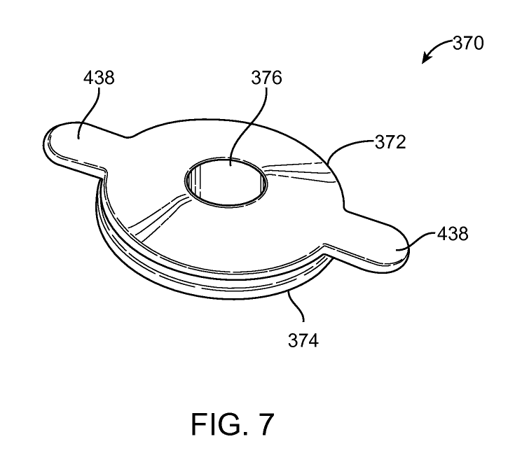

According to another embodiment as shown in FIG. 7, an implant 370 may have flanges 372 and 374 and a lumen 376. At least one of the flanges 372 or 374 may be longer in one radial direction (e.g. non-circular) and the lumen 376 may be relatively longer with a relatively smaller inner diameter. For example, at least one of the flanges 372 or 374 may be oval or have at least one extension or tab 438 along at least one of the flanges. As shown in FIG. 7, the flange 372 may have two tabs 438 along opposite sides of the flange 372, extending in the x- and/or y-directions of the implant 370. The tabs 438 may further bias the implant 370 to move toward the flange 372 as the implant detaches from the surrounding tissue. If both of the flanges 372 and 374 have tabs 438, the tabs 438 may be offset from each other to firmly secure the implant 370 around the tissue. Alternatively, the tabs 438 may be aligned with each other. The implant 370 of FIG. 7 may be particularly suitable for small tubular lumens.

According to one embodiment, the length and width of the tab 438 may range between 3.8 to 6.35 mm. According to another embodiment, the length and width of the tab 438 may be approximately 5.33 mm and 5 mm, respectively. Alternatively or additionally, the flange 14 may have at least one tab 438.

The oblong shape of the implant (such as the oval shape of the flange 352 (as shown in FIG. 5) or the flange 372 with the tabs 438 (as shown in FIG. 7)) may further allow the implant to self-align or rotate according to the direction of flow within the body due to the curvature of the inner lumens. For example, the duodenum has a relatively high degree of curvature around its perimeter, but is relatively flat along its length (e.g. parallel to the direction of flow along the central or longitudinal axis of the duodenum). Therefore, if the flange 352 or 372 (with an oblong shape) is positioned within the duodenum, the flange 352 or 372 will automatically align with the duodenum such the length (e.g. the longest side) of the flange 352 or 372 is parallel to the length of the duodenum and parallel to the direction of flow. This alignment minimizes the amount that the implant 350 or 370 is forced to curve or bend inward to match the inner shape of the duodenum and therefore minimizes the amount that the flange 352 or 372 is pulled or bent away from the flange 354 or 374. Accordingly, the alignment prevents leakage and gaps between the flanges 352 and 354 or 372 and 374, prevents the implant 350 or 370 from dislodging, and maximizes the potential contact between the flanges 352 and 354 or 372 and 374 to cause sufficient pressure to result in necrosis.

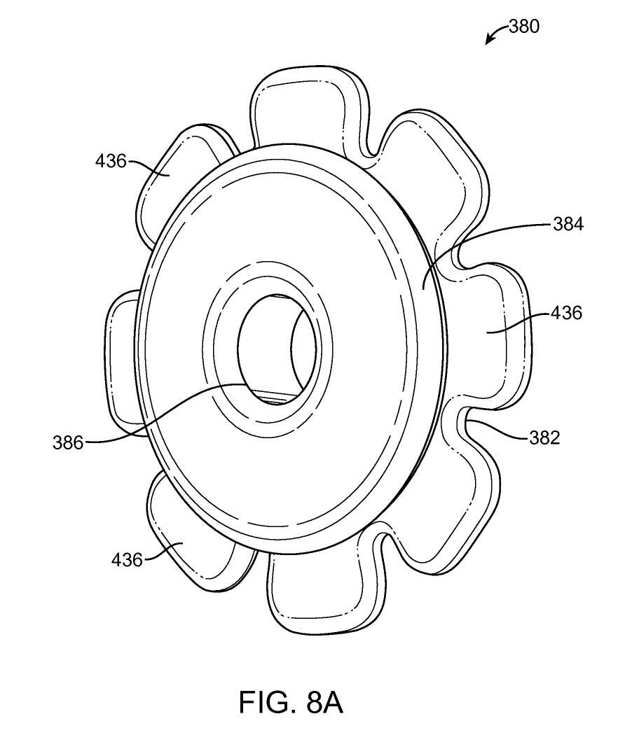

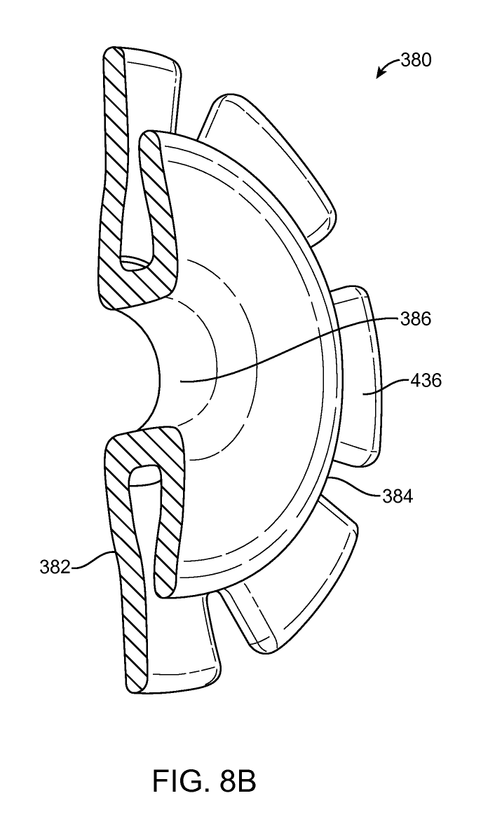

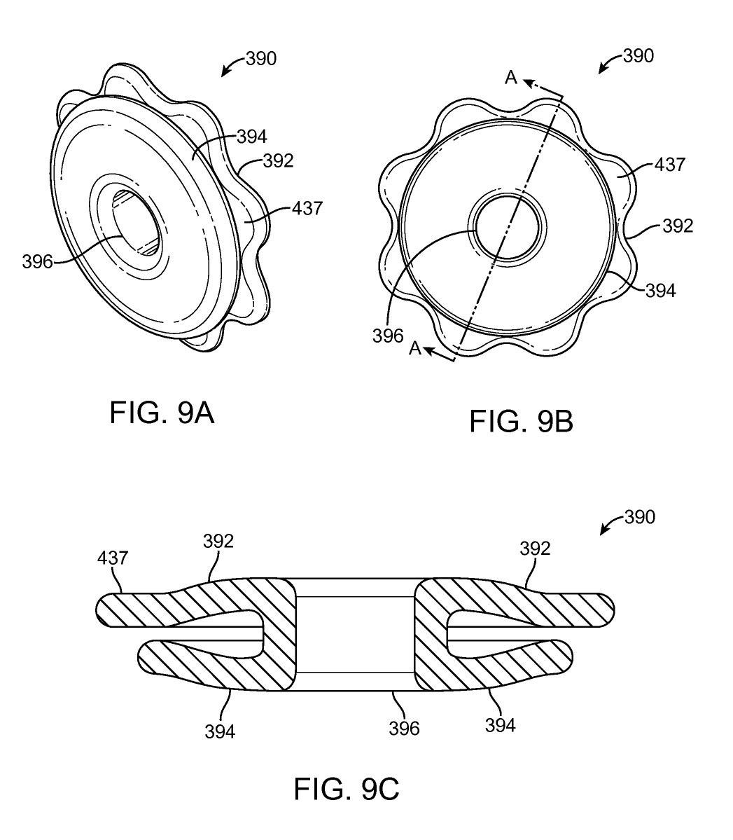

According to another embodiment as shown in FIGS. 8A-8B, an implant 380 may have flanges 382 and 384 and a lumen 386. As shown in FIGS. 9A-9C, an implant 390 may have flanges 392 and 394 and a lumen 396. At least the major axis of the outer diameter of the flange 382 or 392 may be larger than the outer diameter of the flange 384 or 394 and may have an irregular (or patterned) perimeter or edge. The patterned edge of the flange 382 or 392 may allow the flange 382 or 392 to be easily folded, compacted, and compressed for loading, while still biasing the implant 380 or 390 to move toward the flange 382 or 392. Alternatively or additionally, the flange 384 or 394 may have an irregular or patterned perimeter or edge, similar to that which is shown and described for the flange 382 and 392.

As shown in FIGS. 8A-8B, the flange 382 may have at least one slit, gap, or cut-out along the edge or perimeter of the flange 382, creating a “petal” shape. “Petals” or flaps 436 may extend beyond the flange 384 to bias the implant 380 to move toward the flange 382 once the implant 380 is detached or removed from the surrounding tissue. According to another embodiment as shown in FIGS. 9A-9C, the edge of the flange 392 may have wave pattern. Waves 437 may be relatively rounded (as shown in FIGS. 9A-9C) or relatively pointed, according to the desired configuration. The waves 437 may increase the major axis of the diameter from approximately 18.3 to 21.9 mm, as shown in FIG. 9A-9C.

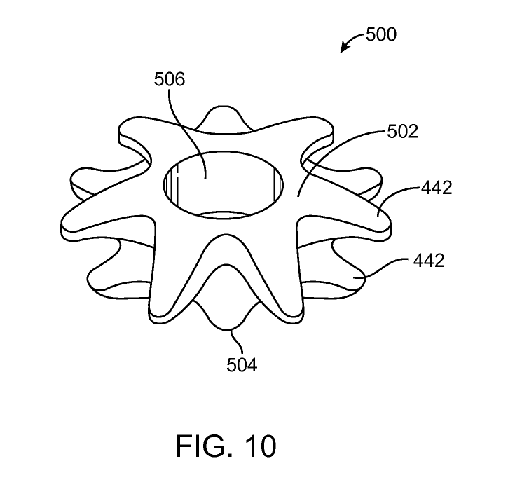

According to another embodiment as shown in FIG. 10, an implant 500 may have flanges 502 and 504 and a lumen 506. The flanges 502 and 504 may be shaped in a curved “star” (with any number of points or radial fingers 442). Depending on the degree of curvature and the relative thicknesses of the flanges 502 and 504, the amount of necrosis (if any) may vary. For example, as shown in FIG. 10, the flanges 502 and 504 may face each other with the radial fingers 442 offset from each other, such that the radial fingers 442 of the flanges 502 and 504 at least partially interlock with each other around the tissue, which may cause point necrosis along certain regions of the tissue. The radial fingers 442 of each of the flanges 12 and 14 may optionally be aligned with each other. However, the flanges 502 and 504 may be configured to at least partially allow fluid or blood flow between the points to prevent complete necrosis. According to another embodiment, only one of the flanges 502 and 504 may have radial fingers 442.

As described further herein, the implant 10 may be configured to be delivered by a delivery device (including but not limited to a catheter, endoscope, or guidewire) in a compact, collapsed, folded, smaller, or compressed configuration 210 through at least one body lumen. The implant 10 may subsequently deployed from the compressed configuration 210 and implanted or secured within the body of the patient. Therefore, the profile (e.g. the diameter) of the compressed implant 10 must be sufficiently small to be delivered with the appropriate delivery device and to reach small areas of the body. For example, the diameter of the tool or working channel of typical echo-endoscopes is between 2.8-3.8 mm. Accordingly, the profile of the compressed implant 10 must be 10 mm or less in order to fit within the endoscope. More preferably, the profile of the compressed implant 10 must be 3 mm or less.

Alternatively, the implant 10 may be delivered outside of an endoscope and, for example, over a guidewire. The endoscope may be positioned next to the guidewire for visualization and guidance during delivery and deployment. Therefore, the size of the implant 10 in the compressed configuration 210 is limited by the size of the body lumens (e.g. the esophagus and duodenum) the implant 10 must be moved through. The implant size may further be limited to allow an endoscope to fit within the body lumens as well. These constraints may also limit the diameter of the implant 10 to be approximately 10 mm or less.

Accordingly, the implant 10 may be minimally sized in order to compress, load, deliver, and deploy the implant 10 easier. The various dimensions, such as material thickness and implant diameter (including lumen diameter), may be minimized in order to allow the implant 10 to be folded or compressed into compressed configuration 210 that is more compressed or smaller.

Alternatively or additionally, the configuration, design, and construction of the implant 10 may allow the implant to be more easily loaded and deformed into the compressed configuration 210. In order to configure the implant 10 into the compressed configuration 210, the flanges 12 and 14 may be folded or bent away from each other along and toward the central axis and compressed (thereby further reducing the outer diameter of the implant 10). Therefore, the implant 10 may incorporate certain features to facilitate collapsing the implant 10 into a smaller size and profile (e.g. the compressed configuration 210) for easier delivery. For example, the flanges 12 and 14 may be constructed out of flexible materials or with specific bend lines, pleats, cuts, discontinuities, voids, and/or cutouts to allow the implant 10 to be easily folded or loaded and to minimize the profile of the implant 10 in the compressed configuration 210. The thickness of the walls of the implant 10 may also vary. Further, different combinations of materials with different material properties (e.g. polymers with different durometers in different elements of the implant 10) may also be used to help with loading or folding and to minimize the profile of the implant 10 during delivery.

Additionally, the actual shape of the flanges may help minimize the profile of the implant in the compressed configuration 210 by allowing the implant to be easily folded and compressed. For example, the implant 500 in the “star” configuration shown in FIG. 10 may facilitate delivery.

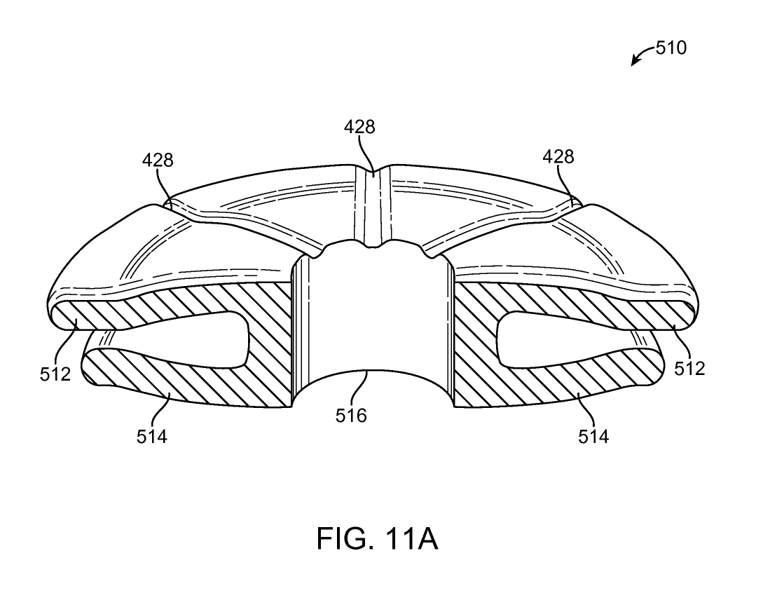

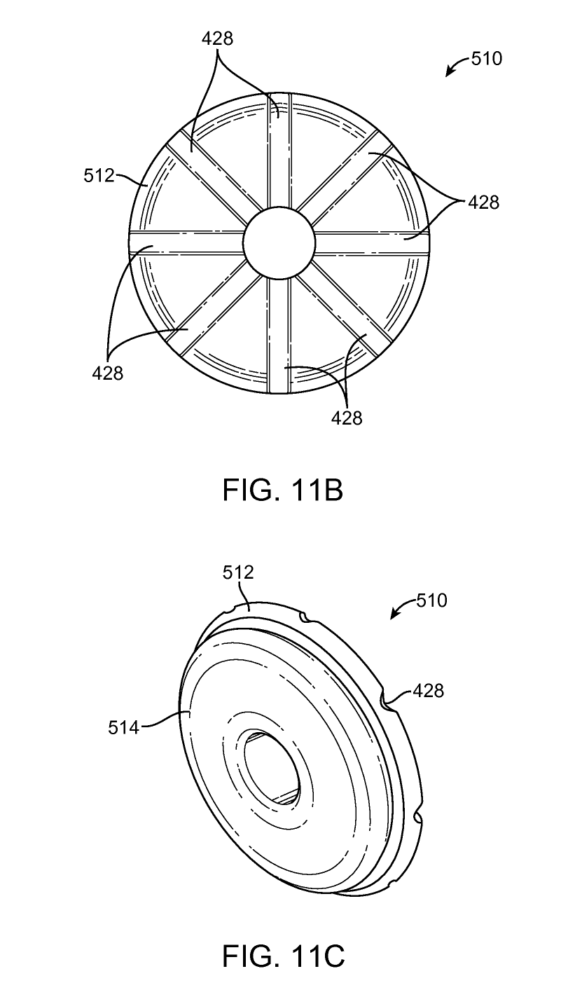

According to another embodiment, a portion of the flanges 12 and 14 may have a textured portion. The textured portion may help with necrosis (as discussed further herein) and/or folding. For example, the textured portion may be at least one groove, bump, and/or slit along at least one of the flanges 12 and 14. For example, FIGS. 11A-11C show an implant 510 may have flanges 512 and 514 and a lumen 516. The implant 510 may have crease or fold lines, slits, or radial grooves 428 along an outside portion of at least one of the flanges 512 or 514 and running perpendicular to the central axis. The textured portion(s) may be along either side of the flanges 512 and 514 such that the textured portion is along the outside of the implant 510 and/or along the inside of the implant 510 (e.g. between the flanges 512 and 514). The radial grooves 428 may allow the implant 510 to differentially fold or pack into a certain shape for easier loading into and deployment from a deployment or delivery device 100. For example, the radial grooves 428 may help the flanges 512 and 514 to fold outward along the longitudinal length or z-axis of the implant 510 in order to compress and deliver the implant 510. Alternatively or additionally, as described further herein, the radial grooves 428 may be located along an inside portion of the flanges 512 and 514 to promote faster necrosis due to additional pressure points or a particular pattern of pressure.

The radial grooves 428 may be located on both flanges 512 and 514 or on only one flange 512 or 514. The radial grooves 428 on the flanges 512 and 514 may be identical or may have a different depth and/or pattern.

According to one embodiment, the radial grooves 428 may be configured to bias the implant 510 to move toward the flange 512 once the implant 510 detaches or ejects from the surrounding tissue. For example, the radial grooves 428 may allow or bias the flange 514 to fold inward in order to move through the aperture in the tissue. Alternatively or additionally, the flange 512 may have a support system, such as additional ribs or bumps, to prevent the flange 512 from collapsing.

The flanges 512 and 514 may have any number and configuration of grooves 428. However, according to one embodiment, at least one of the flanges 512 and 514 may have eight grooves 428, such that the longitudinal axis of the grooves 428 is perpendicular to the central axis. According to one embodiment, the radius of the grooves 428 may range between 0.38 to 0.9 mm. According to another embodiment, the radius of the grooves 428 may be approximately 0.64 mm.

According to various embodiments, the different shapes and configurations of the implant may be combined according to the desired use. For example, the implant may include both the radial grooves 428 and the outer lip 432. Using both the radial grooves 428 and the outer lip 432 may both allow the implant to be loaded easier, as well as stay in place within the body (and preferentially fall out toward the side with the outer lip 432. It is further anticipated that various portions of the configurations and shapes described herein may be combined together.

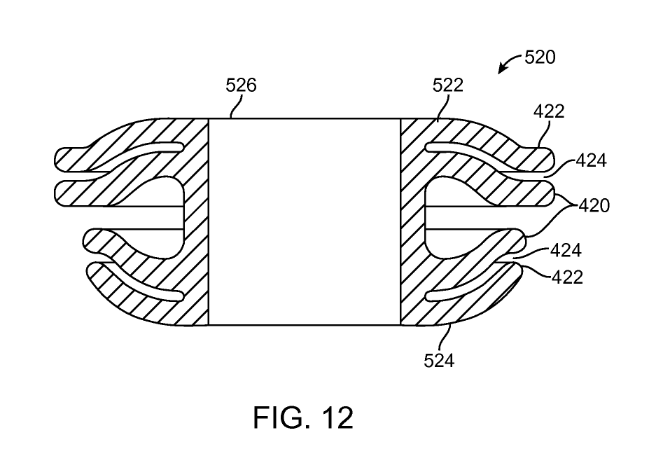

According to one embodiment, FIG. 12 depicts an implant 520 with flanges 522 and 524 and a lumen 526. The implant 520 has a split-flange or double layer design of the implant 520, which may aid in loading the implant 520 while allowing the flanges 522 and 524 to maintain a sufficiently strong hold on the tissue. At least one of the flanges 522 or 524 may be folded back on itself to create at least two layers 420 and 422 of the flange with at least one longitudinal slit, gap, or split 424 (along approximately the x-y plane of the implant 520) in between the layers 420 and 422. For example, each of the flanges 522 and 524 may be separated by the split 424 into at least two components, halves, sides, lips, or layers 420 and 422. The split 424 may follow the curvature or contours of the flanges 522 and 524 (e.g. the angled portion and the base portion of the flanges). The two layers 420 and 422 of each of the flanges 522 and 524 may be identical or may be differently shaped and sized.

In order to stretch out or elongate the flanges 522 and 524 to load the implant 520 (the full process of which is described herein), the outermost layer 422 of each flange may be pulled longitudinally away from each other, which may unfold both the outermost layers 422 and the innermost layers 420. The split 424 in the flanges 522 and 524 may therefore allow the implant 520 to stretch at least twice the length as the implant 520 may stretch without the split 424, allowing the implant 520 to be even more compressed in the compressed configuration 210. The implant 520 may be subsequently collapsed down linearly along the z-axis.

By splitting the flanges 522 and 524, the flanges 522 and 524 may be more easily pulled away from each other (like an accordion) along the z-axis to facilitate folding the implant 520 into the compressed configuration 210. Further, the combination of the layers 420 and 422 may exert an adequate pressure along any tissue therebetween in the deployed configuration 220 and the material thickness of the implant 520 may therefore be thinner to allow the implant 520 to be more easily folded and delivered without compromising the pressure of the flanges 522 and 524 on the tissue.

According to one embodiment, the thickness of each of the sides 420 and 422 may be identical or different. According to one embodiment, the thickness of each of the sides 420 and 422 may range from 0.38 to 0.9 mm. According to another embodiment, the thickness may range from approximately 0.56 to 0.76 mm. The split 424 on the flange 522 may further be identical to or different from the split 424 on the flange 524. According to one embodiment, the split 424 may range from 0.127 to 0.38 mm. According to another embodiment, the split 424 may be approximately 0.25 mm.

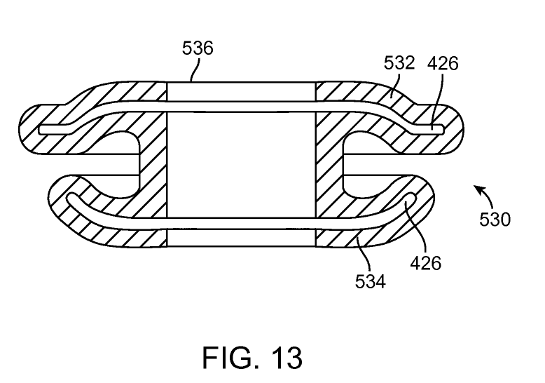

According to another embodiment, FIG. 13 show an implant 530 with flanges 532 and 534 and a lumen 536. The implant 530 with an internal split or gap 426 within the flanges 532 and 534. The gap 426 may allow the two sides of each flange 532 and 534 to at least partially unfold and separate from each other as the flanges 532 and 534 are pulled away from each other. Therefore, the gap 426 may allow the implant 530 to be elongated further along the z-axis and further compressed into the compressed configuration 210. The flanges 532 and 534, however, may maintain an adequate pressure on the tissue secured therebetween due to the combined strength of the two sides of each flange 532 and 534.

The gap 426 may be completely enclosed within the implant 530 or may be at least partially exposed to the outside of the implant 530 (for example, along the lumen 536). As shown in FIG. 13, the gap 426 may not extend completely beyond the edges of the flanges 532 and 534 (e.g. the edges of the flanges 532 and 534 may hold each of the flanges 532 and 534 together). The gap 426 may be hollow (or filled with air) or may be filled with a liquid, such as the surrounding bodily fluid. Both flanges 532 and 534 may have the gap 426 or only one flange 532 or 534 may have the gap 426. Optionally, the gap 426 may extend at least partially along the length of the lumen 536. The gaps 426 on the flanges 532 and 534 may further connect with each other along the length of the lumen 536 (e.g. along the z-axis). According to one embodiment, the thickness of the gap 426 may range from 0.127 to 0.381 mm. According to another embodiment, the gap 426 may be approximately 0.25 mm thick.

The various components of the implant 10, such as the lumen 16 or the flanges 12 and 14, may be elastically deformable, elastically compressible, elastically strainable, compliant, flexible, self-expanding, and/or expandable. Accordingly, the implant 10 may be compressed and/or stretched into a compressed configuration 210 to be delivered into the body and may deploy or expand into the deployed configuration 220, such that the implant 10 may move back into its original configuration. For example, the lumen 16 may be stretched while the flanges 12 and 14 may be bent away from each other. The entire implant 10 may subsequently be compressed in order to be delivered into the body in a highly compressed state in a capsule, for example. Once at least a portion of the implant 10 is released, at least a portion of the implant 10 may automatically expand or recover back into its original configuration, shape, and size. For example, the flanges 12 and 14 may radially expand back to their original diameter and positioning. According to one embodiment, different portions of the implant 10, such as the flanges 12 and 14 or the lumen 16 may have different amounts of flexibility according to the desired use and configuration.

The type and properties of materials used within the implant 10 may affect the amount of applied pressure, as discussed previously. The implant 10 (or various components of the implant 10) may be constructed out of a variety of different materials, according to the desired configuration and use, including but not limited to polymers (e.g. silicone and Teflon), metals (e.g. Nitinol and stainless steel), selected urethanes and polyurethanes, and various combinations of materials (e.g. a metal coated with a polymer). The implant 10 may be made out of silicone, such as PAX silicone, silica, liquid silicone rubber elastomer, dimethyl, and methylhydrogen siloxane copolymer. According to an embodiment, the implant 10 may be constructed out of biocompatible, medical grade silicone. According to another embodiment, the implant 10 may be constructed out of a nitinol weave with a polymer coating.

The stiffness and elasticity of the implant material(s) may vary depending on the desired amount of applied force on the tissue between the flanges 12 and 14. For example, at least a portion of the materials within the implant 10 may be elastomeric and may be constructed out of a material(s) with an appropriate or adequate elasticity to provide the desired applied forces.

Further, the hardness of the implant material may vary according to the application and the desired amount of applied force. According to one embodiment, the material hardness of the flanges may have a shore durometer of ranging between 30 A and 90 A. According to another embodiment, the flanges may have a shore durometer of ranging between 50 A and 80 A. According to one embodiment, the implant may have an approximately 70 A shore durometer.

Further, different portions of the implant 10, such as the flanges 12 and 14, may be configured to be made out of different materials and/or to have different material properties. For example, the material within flange 14 may be more flexible or less hard than the material within the flange 12 in order to bias the implant 10 to fall out toward the flange 12.

At least the exposed portion of the implant 10 may be made out biocompatible materials. The materials may be long-term biocompatible or at least biocompatible for the length of time the implant is intended to be implanted or reside within the body (for example, one to three weeks).

Further, while indwelling or implanted within the body, the implant 10 may be exposed to or submerged in contents of the joined bodily lumens. Therefore, the implant materials may also be compatible with the contents and fluids of the bodily lumens to be joined, as well as the tissues the implant 10 may contact while in the body. For example, the implant 10 used within the biliary system (that may join the duodenum and the gallbladder) may be exposed to chime (the partially digested material exiting the stomach through the pylorus) and bile (the digestive enzyme stored in the gallbladder). The implant 10 implanted within the stomach wall may be compatible with stomach content and fluids.

According to one embodiment, the implant materials may be resorbable or dissolvable over a period of time within the body. Alternatively, the implant 10 may include additional materials that may cause the implant 10 to break up, dissolve, reabsorb, or dematerialize through the application of energy or exposure to selected substances.

Optionally, the implant 10, or portions of the implant 10, may include additives, materials, or components to allow the implant 10 to be easily viewed from outside of the body. For example, the implant 10 may include a radiopaque material, filler, or powder, such as barium sulfate (BaSO₄). The radiopaque filler is standard additive to allow for radiopacity and may allow the implant to be viewed under fluoroscopy. Depending on the desired configuration and type of radiopaque filler, the radiopaque filler may be a powder that is coarsely impregnated (rather than dissolved) into a polymer, which may allow the filler to stay intact as a very fine particulate or suspension. According to one embodiment, the concentration of the radiopaque material may be correlated to the weight of the implant 10. For example, 10-20% of the weight of the implant 10 may be due to the filler. More specifically, the concentration of barium sulfate may be approximately 20% of the weight of the implant 10. The filler may be both non-toxic and bio-compatible.

Alternatively or additionally, the implant 10 may include features, such as radiopaque markers, to aid in visualization. The radiopaque markers may be mechanical features molded into the implant 10 which may include, but are not limited to, a Nitinol ring, stainless steel components, or a braided stainless steel cable. Alternatively or additionally, the implant 10 may include platinum radium markers. The markers may optionally be molded into the inside of the implant 10.

Alternatively or additionally, the implant 10 may be coated with a variety of coatings to affect how the implant 10 is deployed. For example, the implant 10 may be coated with a lubricious or parylene coating to allow the implant 10 to be ejected from the capsule and deployed easier.

The implant 10 may be colored to allow the user to easily identify the proper orientation of the implant 10. For example, the implant 10 may have a color differential between the flanges 12 and 14 to allow the user to easily and correctly orient the implant 10 into the loading device or delivery device 100, such that the first flange 12 is deployed within the distal cavity and the second flange 14 is deployed within the proximal cavity. Further, once the implant 10 is deployed within the body, the color may allow the orientation, the presence of the implant 10, and the correct deployment to be easily viewed with an endoscope.

According to one embodiment, reinforcement structures, such as a Nitinol ring or rim, may be incorporated into the implant 10 to minimize the overall thickness and bulk of the implant 10, while maintaining the structural integrity of the implant 10. For example, with the reinforcement structures, the implant 10 may have a spring force between the flanges 12 and 14 and cause necrosis, but the silicone walls of the implant 10 may be relatively thinner. The reinforcement structure may be formed from a small diameter wire to minimize the profile of the implant 10 during delivery through or adjacent to an endoscope or delivery device. The small wire may also keep the strain levels within the elastic range during the largest expected deformations of the implant 10.

Nitinol may be suitable as a reinforcement structure and may be configured for use in the super-elastic regime. Alternatively or additionally, Nitinol may be used in the shape-memory regime and “taught” to assume the desired deployed configuration 220 once it reaches body temperature. For example, the implant 10 may be collapsed and maintained below body temperature to facilitate the compressed configuration 210 for delivery through or adjacent to an endoscope or delivery device. The resulting phase change of the implant 10 (and the Nitinol) rising to body temperature causes the Nitinol (and the implant 10) to assume the desired deployed configuration 220.

According to another embodiment, the implant 10 may include various other additions or components. For example, flow control features, such as a valve, may be incorporated into or added to the lumen 16 of the implant 10 to control the flow or movement or material within the lumen 16. The valve may include, but is not limited to, a one-way valve, a pressure relief valve, and/or a full seal. The valve may be mechanically or instrincally controlled within the body. According to another embodiment, the valve may be externally controlled and may include electronic components to control the flow within the implant 10.

The valve may, for example, only allow material to flow in one direction through the lumen 16 (e.g. a “one-way flow” valve). The one-way flow valve may include, but is not limited to, a duck-bill valve, a flap valve, a sleeve valve, a biscuspid-valve, a tricuspid-valve, or a n-cuspid valve (where “n” is any number).

Alternatively or additionally, the valve may only allow material to flow when the pressure differential across the implant 10 exceeds a particular value (e.g. a “pressure relief” valve). The pressure relief valve may therefore prevent material flow when there is insufficient pressure across the implant 10. The pressure relief valve may include, but is not limited to, a pop-off valve or a blow-off valve. According to another embodiment, the valve may be a slit (or multiple slits at different orientations) in the implant 10 that may open and close depending on the pressure differential between the connected lumens. For example, with sufficient pressure from the gallbladder, the valve may open and allow material (e.g. gallstones) to move through the valve and into the duodenum. According to one embodiment, the valve may be both a pressure relief valve and a one-way flow valve.

Alternatively, the valve may restrict or completely prevent material from moving or flowing through the lumen 16 (e.g. a full seal or “plug”). A plug may be used when the flow of material is not needed or desired, but the formation of a fistula around the implant 10 and/or the progression of pressure necrosis occurring between the flanges 12 and 14 is desirable and/or beneficial.

According to one embodiment, the valve may be integrally formed with or incorporated into the implant as a single assembly. According to another embodiment, the valve may be a separate insert or add-on that can optionally be added onto or removed from the implant 10, prior to delivery, during delivery or deployment, or in situ. For example, the implant 10 and the valve may include mating features to securely attach, seal, or connect with each other, which may allow the valve to be selectively installed and removed from the implant 10. The insertable and removable valve may enable clinicians to adjust the course of treatment without removing the shunt. For example, the clinician may adjust the opening pressure of the valve, stop the flow of material (with a plug), or remove the implant 10 in order to allow tools to be inserted through the lumen 16 (which may allow a separate procedure to be performed, using the implant 10 to provide access). According to one embodiment, the valve may be inserted, removed, or changed endoscopically.

According to another embodiment, the implant 10 may be responsive to body temperature.

According to still another embodiment, the implant 10 may include a tracking mechanism, tag, device, or chip to allow the doctors to monitor the location of the implant 10. For example, the tag may allow the doctors to know if the implant 10 is still attached to the tissue, misplaced, detached from the tissue (due to tissue necrosis or other means), or moved at least partially through the digestive system.

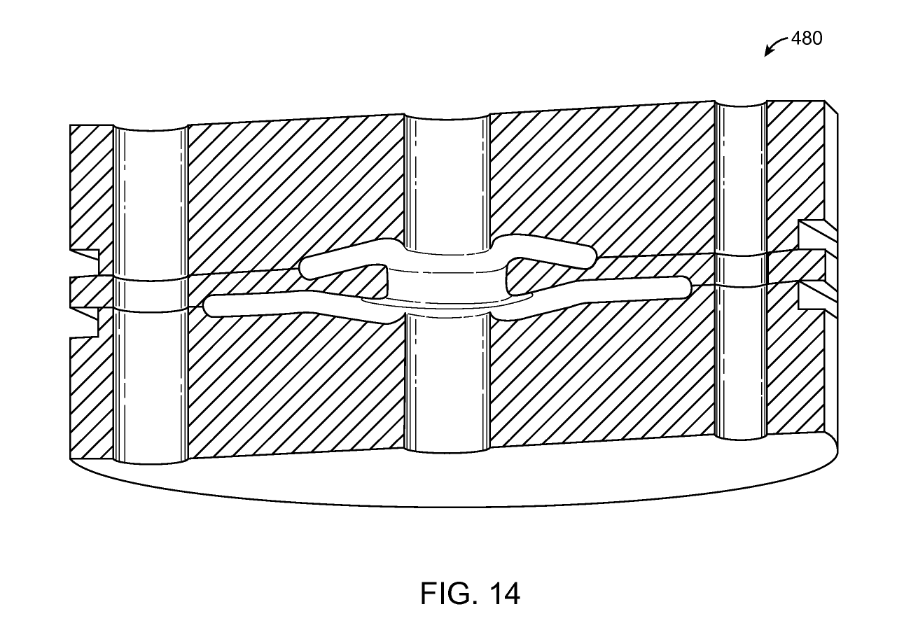

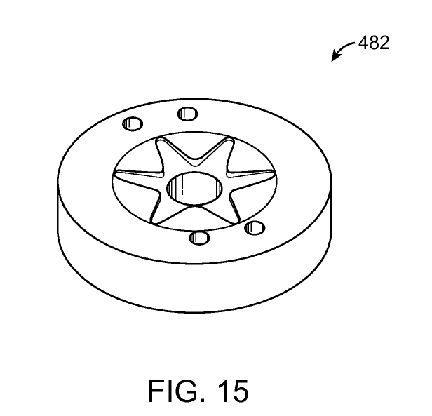

The implant 10 may be manufactured through a variety of different methods and techniques. For example, the implant 10 may be single-part molded to simplify the manufacturing procedure and to eliminate the need for internal skeletal reinforcements. As shown in FIG. 14, an implant mold 480 may be used to form the implant. The implant mold 480 may have an inner cavity with the desired shape and size of the implant. As shown in FIG. 14, the implant mold 480 may include multiple layers to create the exact desired configuration of the implant. FIG. 15 depicts another embodiment of an implant mold 482, which may be used to produce the implant 500 shown in FIG. 10.

According to one embodiment, at least a portion of the implant 10 may be constructed out of silicone. The silicone may be cast into the desired shape using a two-part silicone mixture within the implant mold 480 or 482. The uncured silicone may optionally be pressure- and/or vacuum-drawn into the implant mold in order to further optimize the results or production time. Once the implant mold 480 or 482 has been filled with uncured silicone, the silicone may being to cure. Depending on the type of silicone, the curing may occur at room temperature. However, the temperature may be elevated in order to accelerate the curing process. For example, the implant 10 may be cured at 100° C. for one hour.

In order to incorporate a metallic material, such as Nitinol, into the implant 10, the metallic material may be formed from a small diameter wires and braided or arranged in order to incorporate the lumen 16 and the flanges 12 and 14 in the deployed configuration 220.

The implant 10 may be constructed in multiple parts or components. For example, the lumen 16 may be constructed discretely or separately from the flanges 12 and 14. The lumen 16 and the flanges 12 and 14 may be assembled before delivery into the body. Alternatively, the components may be delivered separately or detached and subsequently assembled within the body. If the implant components are delivered separately to the implantation sight, due to the smaller size and profile of the individual components (compared to the size of the entire implant 10), the components may be passed through a relatively small working channel, such as a 3.7 mm working channel of an endoscope. The lumen 16 and the flanges 12 and 14 may snap together directly or to a reinforcing receiver.

The various embodiments, components, and characteristics of the implant, as shown, for example, in FIGS. 1-13 may be interchangeable, added, or subtracted from an implant according to the desired configuration. For example, although implant 10, flanges 12 and 14, lumen 16, base portion 406, and angled portion 408 may referred to, it is anticipated that the characteristics of any of the embodiments may be used together within an implant. For example, the characteristics and configurations of the implants 10, 300, 310, 320, 330, 340, 350, 360, 370, 380, 390, 500, 510, 520 or 530, the flanges 12, 302, 312, 322, 332, 342, 352, 362, 372, 382, 392, 502, 512, 522 or 532, the flanges 14, 304, 314, 324, 334, 344, 354, 364, 374, 384, 394, 504, 514, 524 or 534, the lumens 16, 306, 316, 326, 336, 346, 356, 366, 376, 386, 396, 506, 516, 526 or 536, the base portions 406 or 337, or the angled portions 408 or 338 may be interchangeable.

The various embodiments of the implant may be folded into a particular configuration, loaded into a delivery device, and delivered and deployed within the body. Although the implant 10 and its respective flanges 12 and 14 and lumen 16 are referred to, it is anticipated that any of the implants 10, 300, 310, 320, 330, 340, 350, 360, 370, 380, 390, 500, 510, 520 or 530 and their respective components may be used in conjunction with the loading device and methods and the delivery device and methods described further herein.

### Loading Devices and Procedures

Referring generally to the figures, the shunt or implant 10 may be folded, prepared, packaged, or loaded into a particular configuration in order to fit within the deployment or delivery device 100 and to be properly delivered and deployed within the body of the patient. The method and apparatus described herein may be used with a variety of medical procedures, including but not limited to surgical procedures or endoscopic procedures. In order to properly deploy the implant 10 into the expanded or deployed configuration 220 within the body of the patient, the implant 10 may be folded and compressed into the compressed configuration 210 and loaded into the delivery device 100 outside of the body of the patient by using loading devices. Once the delivery device 100 is advanced to the implantation site within the body of the patient, the implant 10 may be deployed from the compressed configuration 210 to the deployed configuration 220, such that the anchoring lip or flange 12 (e.g. the first flange) is deployed within a proximal cavity (e.g. a first cavity or the duodenum) and the anchoring lip or flange 14 (e.g. the second flange) is deployed within a distal cavity (e.g. a second cavity or the gallbladder) and at least one tissue wall is secured between the flange 12 and the flange 14. The apparatus and methods described herein may be used with a variety of different implants or shunts, such as a cholydoco-duodenal shunt.

In order for the implant 10 to be fully functional within the body of the patient, the implant 10 must be properly delivered and deployed. However, the performance of the delivery system and proper deployment may depend on the correct loading of the implant 10 into the delivery device 100. In order to properly deliver the implant 10, it may be beneficial to minimize the cross-sectional area (e.g. the profile along the x-y plane) of the implant 10. Therefore, the implant 10 may be loaded in such a way as to minimize the cross-sectional area of the implant 10. However, simply packing the implant 10 as small as possible may not provide the proper configuration of the implant 10. In order to allow the delivery system to maintain control of the implant 10 throughout the delivery and deployment process, the implant 10 may be loaded with a particular method and into a particular configuration.

In order to load the implant 10 into the delivery device 100 to eventually be deployed within a patient, the implant 10 may be folded from the deployed configuration 220 to a folded or pleated configuration 200, such that the flange 12 is at least partially pulled apart, extended, or separated from the flange 14 in a lengthwise direction along a central axis of the implant 10. The flanges 12 and 14 may be pleated in order to properly fold the implant 10 for deployment.

From the pleated configuration 200, the implant 10 is additionally radially compressed along the lengthwise direction and may be at least partially folded into the compressed configuration 210. A variety of mechanisms and devices may be used to lengthen, compact, and compress the implant 10, as well as secure the implant 10 in the compressed configuration 210. The implant 10 may be stored, sold, shipped and/or incorporated into the delivery device 100 in the compressed configuration 210.

Once the implant 10 has been secured into the compressed configuration 210, the implant 10 may be drawn into and secured within a generally cylindrical container, cup, or capsule 110 within the delivery device 100. The delivery device 100, containing the implant 10 in the compressed configuration 210, may be advanced into a first cavity of a patient (e.g. the duodenum) and at least partially into a second cavity (e.g. the gallbladder). Once the delivery device 100 is properly positioned, the implant 10 may be expanded into the deployed configuration 220, such that the flange 14 deploys within the second cavity (e.g. the gallbladder) and the flange 12 is subsequently deployed within the first cavity (e.g. the duodenum).

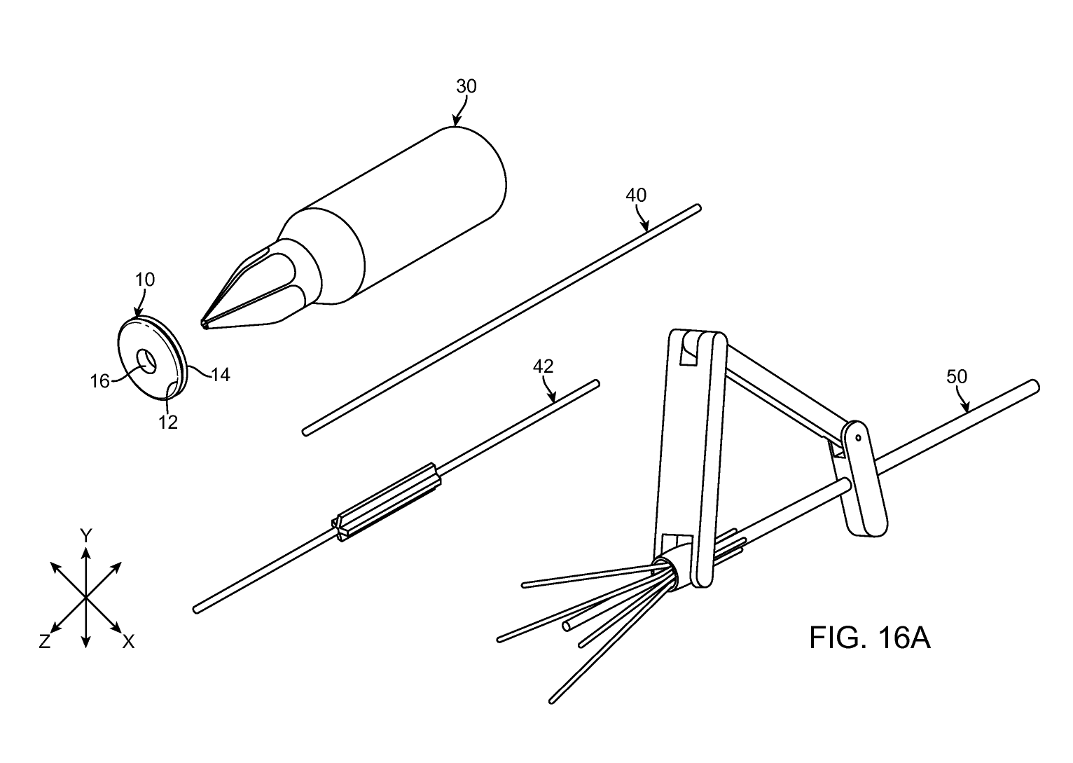

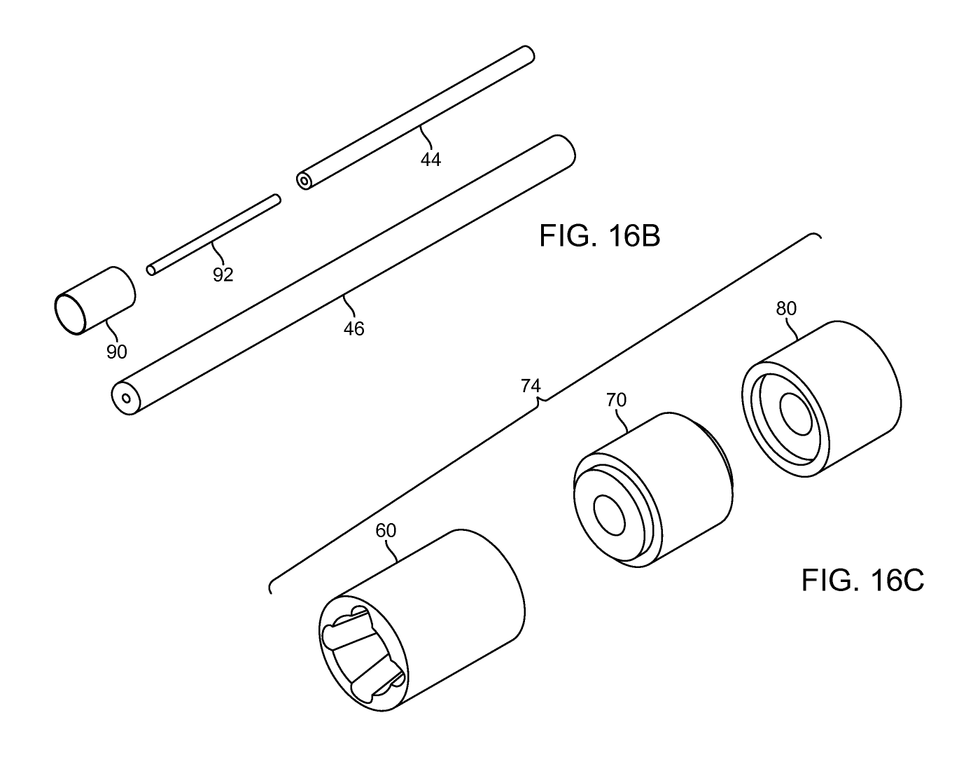

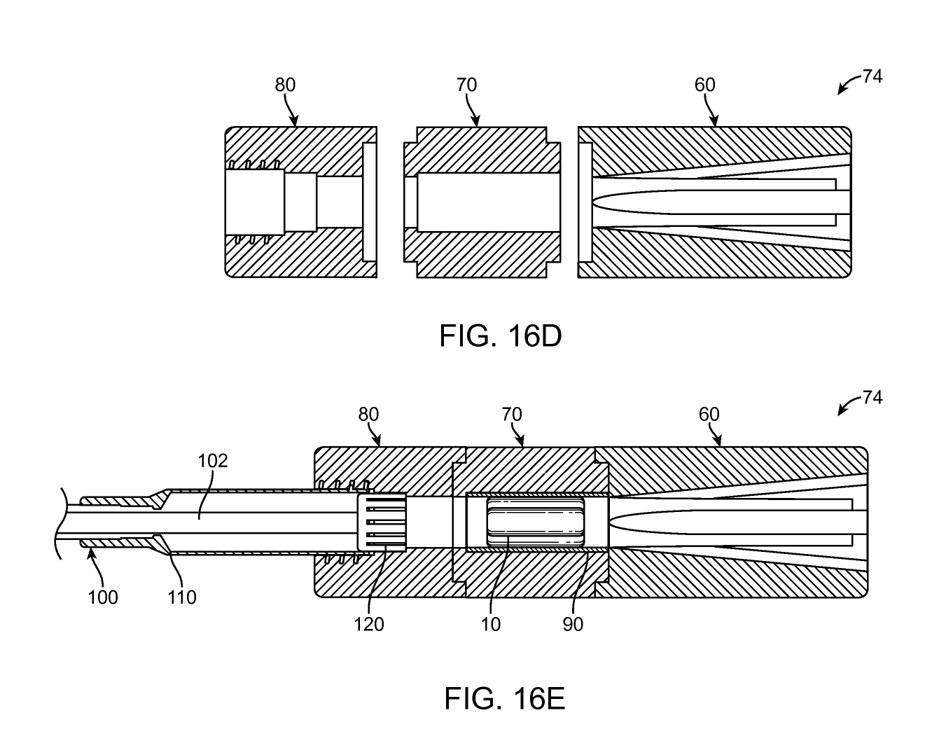

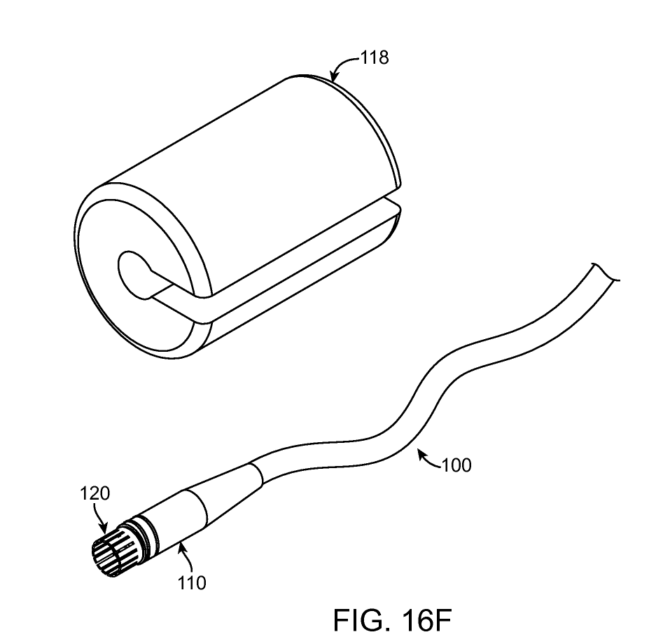

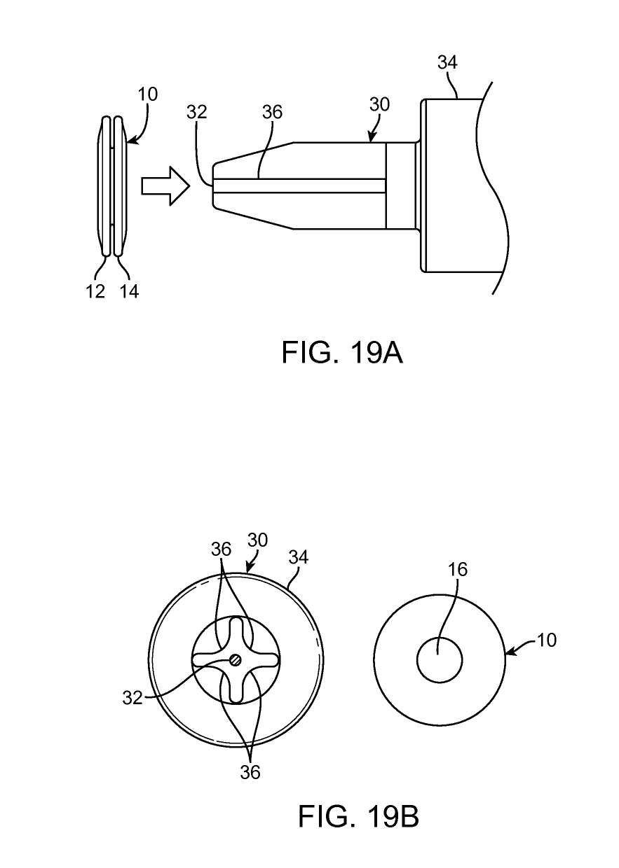

A variety of instruments or tools may be used in order to load and deploy the implant 10. For example, FIGS. 16A-16D depict exemplary instruments that may be used to load and/or deploy the implant 10 and to manipulate the implant 10 into a variety of configurations, such as the loading devices and the delivery device 100. In one embodiment, the implant 10 includes two flanges 12 and 14 (see FIG. 19A). The flange 12 may be deployed against a wall of the duodenum. The flange 14 may be deployed against a wall of the gallbladder. It is anticipated, however, that the implant 10 may be oriented into any configuration during loading, deployment, delivery, and use.

The loading devices and methods may facilitate highly compressing the implant 10 to minimize the profile of the implant 10 during delivery. Further, the loading devices and methods control and organize the configuration and orientation of the implant 10 in the compressed configuration 210 to allow for reliable and predictable delivery and deployment at the desired site within the body.

FIG. 16A shows various loading devices. The loading devices may include an expanding mechanism or tool, such as a dilator, mandrill, or conical rod 30. The conical rod 30 may be used to expand or stretch the lumen 16 of the implant 10 and separate the flange 12 and the flange 14. The lumen 16 may define a passageway along the central axis of the implant 10. An implant compressor 50 may be used to compress and pleat or fold the implant 10.

Miscellaneous centering devices, mandrels, pushrods, and ramrods may also be used to improve the control of the loading process of the implant 10. For example, a centering mandrel 40 and a finned mandrel 42 may be used to support and/or align the various components and tools within the loading and deploying system. For example, the centering mandrel 40 and the finned mandrel 42 may be used to align the implant 10 and the deployment device.

FIG. 16B shows additional loading devices, such as a loading tube or tool 74, which may include any combination of an implant entry section or tapered tool or tube 60, a middle transfer section or sheath holder 70, and/or a capsule loading section 80. A reversible sleeve or sheath 90, which may be positionable in the sheath holder 70, may be used to secure the implant 10 into a compressed configuration. The sheath 90 may be held in place with the sheath holder 70. A balloon shaft guide or tube 92 may be inserted into the lumen 16 of the compressed implant 10 in order to provide a passageway within the implant 10 for additional delivery or loading components to move through, such as a balloon catheter. Pushing mechanisms or devices, such as but not limited to a small ramrod or rod 44 and a large ramrod or rod 46, may be used during the loading and delivery process to help move various components of the system. For example, the rod 44 or 46 may be used to push the implant 10 in the pleated and/or compressed configurations between the sections of the loading tool 74 and into the delivery device 100.

As shown in FIG. 16F, the delivery device 100 may be used to deliver and deploy the implant 10 into a patient. The delivery device 100 is shown to include the capsule 110 to contain the implant 10 during delivery and deployment. The capsule 110 may include an expandable (and collapsible) positioning mechanism, implant holder, or positioner 120, which may be used to grasp a portion of the implant 10 within the capsule 110, such that the implant 10 is deployed properly. The implant positioner 120 may be moveable in and out of the capsule 110 to allow the implant 10 to be loaded. The implant positioner 120 may at least partially retract within the capsule 110 to secure the implant 10 within the delivery device. While the implant 10 is being loaded into the delivery device 100, a loading grip 118 may be used to grasp a portion of the delivery device 100, such as the capsule 110. The capsule 110 may be located along a distal end of the delivery device 100.

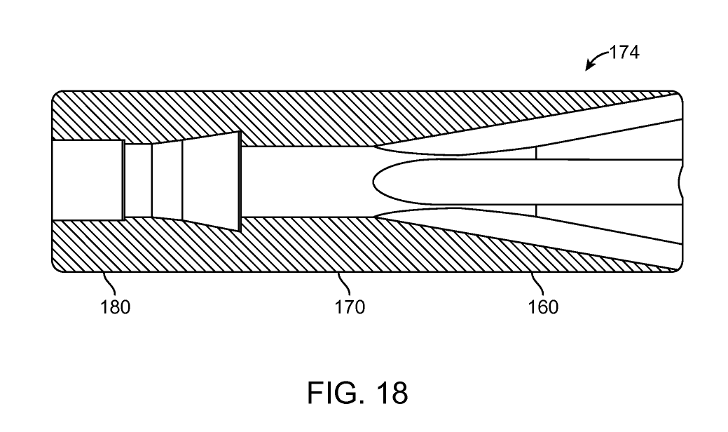

As shown in FIGS. 18-20, according to one embodiment, the lumen 16 of the implant 10 is expanded with an expanding mechanism (such as the conical rod 30) in order to properly fold and compress the implant 10. The conical rod 30 may be primarily used to spread open the lumen 16 and at least partially flatten (along the z-axis) the implant 10. As described further herein, the lumen 16 may include the first flexible flange 12 and the second flexible flange 14. As shown in FIG. 19A, a tapered or pointed end or tip 32 of the conical rod 30 may be inserted or moved at least partially through into the lumen 16 and the implant 10 is stretched at least partially around or over the conical rod 30, such that the tip 32 on the distal end of the conical rod 30 extends through the lumen 16 of the implant 10. According to one embodiment, the tip 32 may have a hole or aperture extending at least partially through the conical rod 30 to accept other tools during the loading process, such as the centering mandrel 40.

The conical rod 30 may be at least partially tapered to gradually expand or dilate the lumen 16. As shown in FIGS. 19A and 19B, the tip 32 may be surrounded by longitudinal grooves or flutes 36 along the central axis, defining ribs or protrusions therebetween. The lumen 16 may at least partially overlap over a portion of the flutes 36. Although four flutes 36 are shown in FIGS. 19A and 19B, the number and configuration of the flutes 36 may be altered according to the desired folding pattern of the implant 10. The conical rod 30 may be held in place with a handle 34 on the opposite end as the tip 32. As shown in FIG. 19A, at least a portion of the fluted section may have a constant diameter. Accordingly, the conical rod 30 may flatten sections (between the flutes) of the expanded lumen 16.

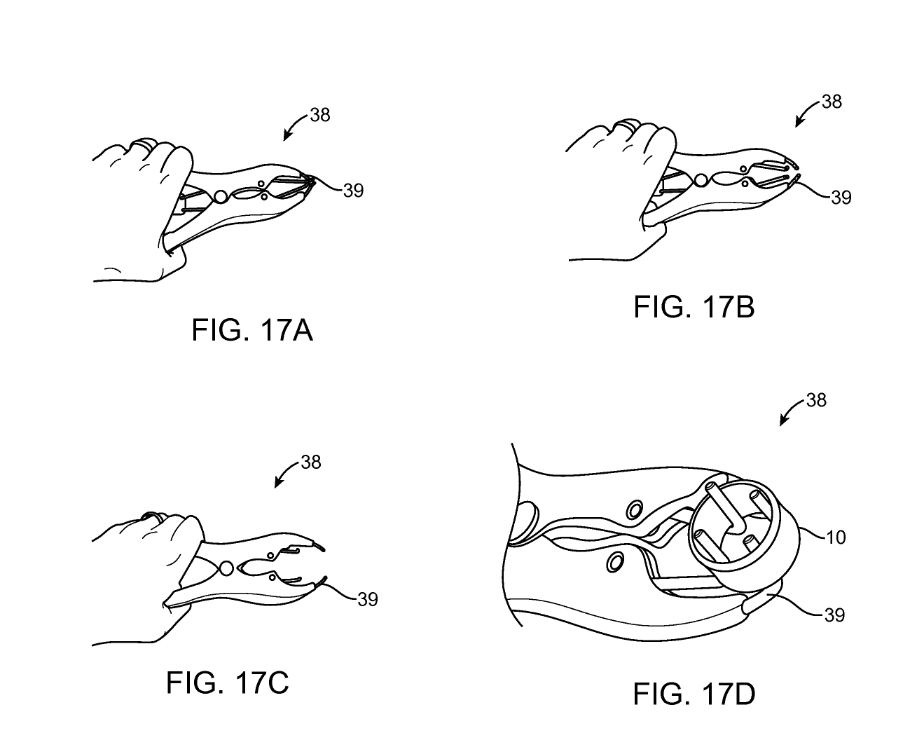

According to one embodiment, the conical rod 30 may have no moving parts and may be considered a “static” tool. However, it is anticipated that an expanding mechanism, such as a spreading dilator tool or actuatable tool 38, with movable parts may be used to spread open and flatten the implant 10, as shown in FIGS. 17A-17D. After loading the implant 10 onto the actuatable tool 38, the actuatable tool 38 may actuate a jaw or claw 39, which may expand the claw 39 radially outward, thereby opening and flattening the implant 10 (as shown in FIG. 17D). FIG. 17A depicts the claw 39 closed, FIG. 17B depicts the claw 39 partially open, and FIG. 17C depicts the claw 39 fully open.

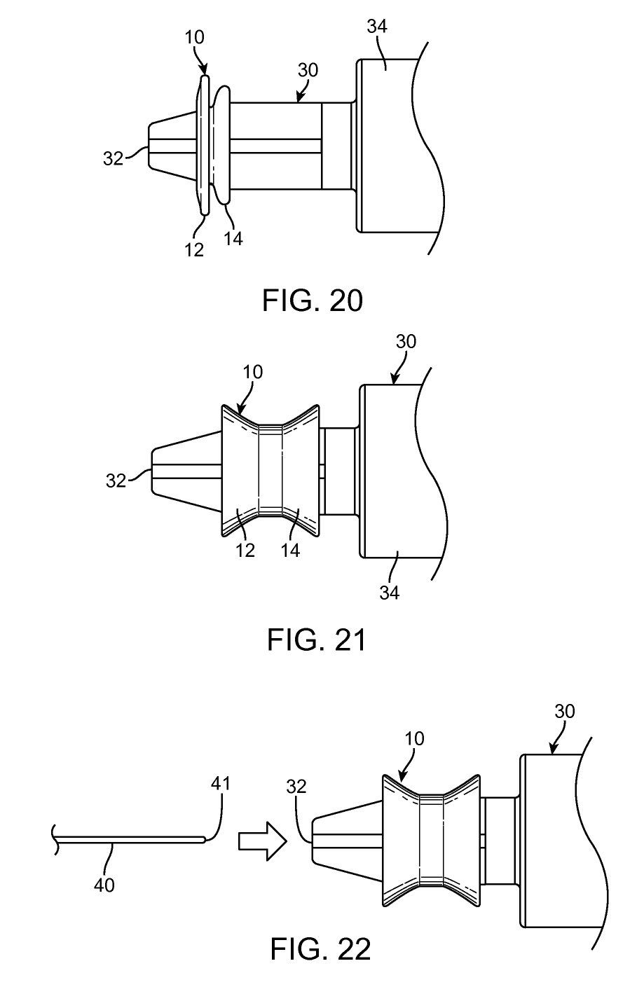

Referring to FIGS. 20 and 21, the implant 10 is shown as being expanded or slide over the conical rod 30 until the implant 10 rests at least partially over the constant-diameter fluted section of the conical rod 30. Due to the flexibility of the implant 10, at least the lumen 16 of the implant 10 may assume the outer shape of the conical rod 30. In order to obtain a particular folding configuration or pattern, the implant 10 may be positioned on the conical rod 30 such that the flange 12 is closer or proximal to the tip 32 and the flange 14 is closer or proximal to the handle 34. As the implant 10 is expanded, the flanges 12 and 14 may be flattened, extended, or moved away from each other, such that the flange 12 is moved toward the tip 32 and the flange 14 is moved toward the handle 34. As the flanges 12 and 14 are moved outward, the length of the implant 10 increases while the width of the implant 10 decreases. Since the outer diameter of the conical rod 30 is larger than the inner diameter of the lumen 16 (in the deployed configuration 220), the flanges 12 and 14 will maintain their position due to the elastic properties of the implant 10. Further, due to the flexibility and elasticity of the implant 10, the implant 10 may conform to or assume the outer shape and size of the conical rod 30.

The conical rod 30 or the actuatable tool 38 may accommodate subsequent compressing and folding of the implant 10 with the implant compressor 50. For example, the flutes 36 incorporated into the tip 32 and into the constant-diameter middle section of the conical rod 30 may accommodate the implant compressor 50. Alternatively, the spaces between the spreadable claw 39 of the actuatable tool 38 may accommodate the implant compressor 50.

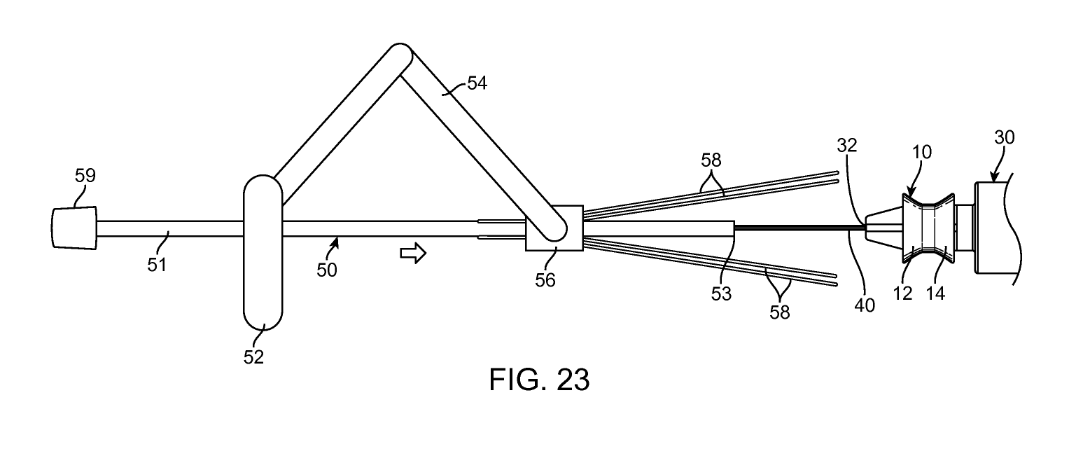

As shown in FIG. 22, the centering mandrel 40 is inserted into the tip 32 of the conical rod 30 (the conical rod 30 may include a hollow inner portion or passageway). As shown in FIG. 23, the centering mandrel 40 may also be inserted within a central tube 51 along a central longitudinal axis of the implant compressor 50 and may be used to align the implant compressor 50 to the conical rod 30 and the implant 10. The centering mandrel 40 may include a stopper to prevent the centering mandrel 40 from moving or sliding out of the conical rod 30.

The centering mandrel 40 may be used to define and maintain the central axis of the implant 10 during the loading process, particularly while the implant 10 is being folded and compressed into the pleated configuration 200 or the compressed configuration 210 and is being held in the minimum profile configuration.

According to one embodiment, the centering mandrel 40 may be lubricious. For example, the centering mandrel 40 may be constructed out of a lubricious material, including but not limited to PTFE, polished stainless steel, or LDPE. Alternatively or additionally, the centering mandrel 40 may include an additional component, coating and/or treatment to be lubricious. For example, a lubricous coating or treatment (e.g. PTFE or Parylene) may be applied to the outer surface of the centering mandrel 40. This may be particularly useful if the centering mandrel 40 is aluminum, mild steel, or stainless steel.

According to another embodiment, a rounded leading tip 41 of the centering mandrel 40 may be rounded or radiused, which may facilitate the insertion of the centering mandrel 40 into other components, such as the central lumen of the conical rod 30. The leading tip 41 may further prevent scratching, scoring, or tearing of the components (e.g. the implant 10).

The centering mandrel 40 may be long enough to remain in place during various steps of the loading process. For example, the centering mandrel 40 may be approximately 20-25 cm long (however, it is anticipated that longer or shorter centering mandrels may be used). The diameter of the centering mandrel 40 may accommodate components intended to be inserted through the implant 10, including but not limited to guidewires, balloon shafts, and balloon shaft guides. The centering mandrel 40 may also be sized to fit within other components, such as the tip 32 of the conical rod 30. According to one embodiment, the diameter of the centering mandrel 40 may range between 1.27 to 3.81 mm.

Referring back to the loading process and to FIGS. 23-25, the implant compressor 50 may be used to radially compress, fold, or constrain longitudinal or lengthwise portions or regions of the expanded implant 10 (along the z-axis of the implant 10), such that the implant 10 may be folded into a particular configuration or pattern with a minimal profile and organized folds. The implant compressor 50 may additionally maintain these longitudinal compressive forces to allow the implant 10 to be moved and manipulated while the folds are maintained. For example, the implant compressor 50 may help maintain the folds while the implant 10 is being removed from the conical rod 30, preparing the implant 10 for insertion into the loading tool 74. The implant compressor 50 may also be used to push the compressed and folded implant 10 into the loading tool 74. In other embodiments, another mechanism may be used to fold or pleat the implant 10.

Proper deployment of an implant 10 within the body of a patient may depends on the position of the implant 10 within the delivery system. Therefore, throughout the performance of the operations of the implant compressor 50, the implant compressor 50 may maintain the organization, control, and configuration of the compressed implant 10, which may prevent the implant 10 from becoming jammed or wadded while being pushed into the loading tool 74.

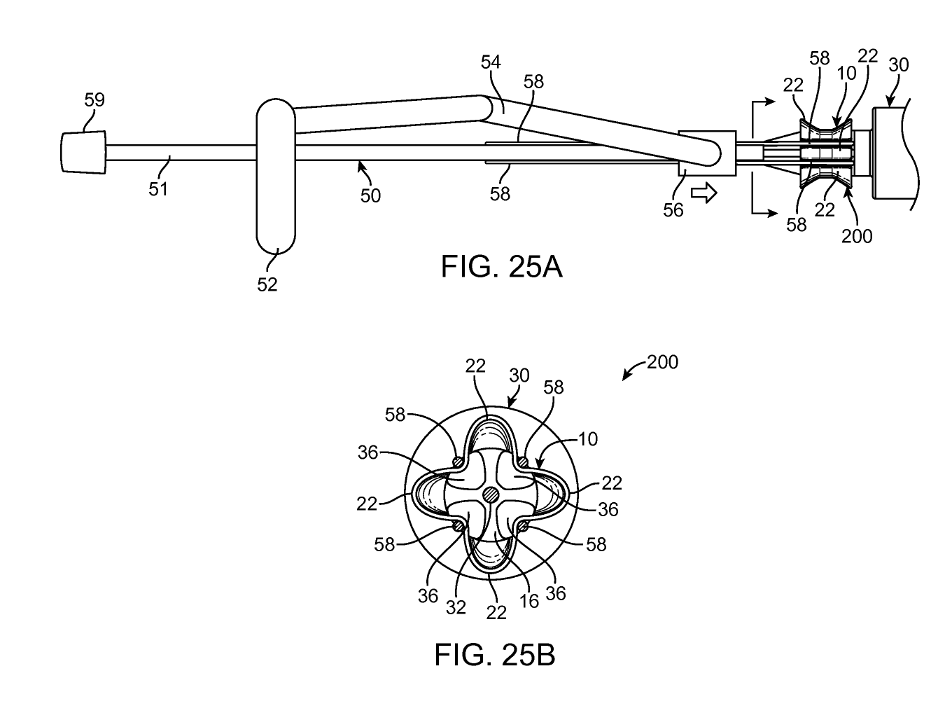

The implant compressor 50 may include movable rods, angled spring elements, compression fingers, or branches 58 which may radially close down around the implant to compress the implant. The branches 58 may be generally flat against or parallel to the central axis of the implant compressor 50 and may radially expand outward from the central axis. The branches 58 may correlate directly with and depend on the size, configuration, and number of the flutes 36 along the conical rod 30, pushing and compressing longitudinal or lengthwise portions of the implant 10 into the flutes 36 to form implant pleats 22 and compress the implant 10 into the pleated configuration 200. For example, the branches 58 may at least partially fit within the flutes 36, sandwiching the implant 10. The compressed lengthwise portions of the implant 10 may correspond with at least one flute 36 along the conical rod 30. The number of pleats 22 along the implant 10 may directly correlate to the number of the branches 58 and the flutes 36, as shown in FIG. 25B. The pleats 22 may be generally symmetrical about the outer circumference of the implant 10. A sliding finger compressor or sliding portion 56 of the implant compressor 50 may slide over the branches 58 and toward the distal end of the implant compressor 50, causing the branches 58 to radially compress inward. According to one embodiment, the sliding portion 56 may have a ring shape with an inner diameter sized to accept the central tube 51 and the branches 58. An actuation lever or clevis arms 54 may advance or retract along the length of the implant compressor 50 to move the sliding portion 56. An anchor or fixed portion 52 may attach to the central tube 51 and serve as a point of leverage for the arms 54.

The central tube 51 of the implant compressor 50 may serve as a primary structural element for the implant compressor 50. The implant compressor 50 may also include an implant ejector 53 which may push the compressed implant 10 out of the implant compressor 50 and into the loading tool 74. A stopper or plunger 59 may be attached to a proximal end of the implant compressor 50 and may be further attached to a rod or tube that slides inside of the central tube 51 to advance the implant ejector 53.

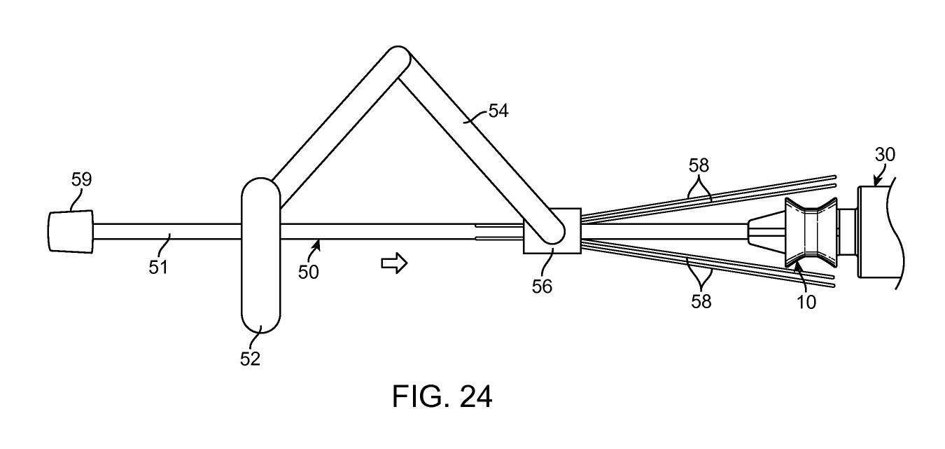

FIG. 23 depicts the implant compressor 50 before actuation, with the sliding portion 56 fully retracted toward the proximal end of the implant compressor 50. FIG. 24 depicts partial actuation of the implant compressor 50, where the sliding portion 56 partially advanced over the branches 58, forcing the branches 58 to radially compress inward over the implant 10. FIG. 25A depicts full activation of the implant compressor 50, in which the sliding portion 56 is advanced to the end of travel, further forcing the branches 58 radially inward to their travel limit (e.g. near, touching, or approximately parallel to the central tube 51).

In order to remove the dilated implant 10 from the conical rod 30, the implant compressor 50 may move or advance along the centering mandrel 40 toward the implant 10 and the conical rod 30, as shown in FIGS. 23 and 24, until the implant ejector 53 touches the tip 32 of the conical rod 30 and the branches 58 are surrounding or over the implant 10 (as shown in FIG. 24). In order to compress the implant 10, as shown in FIG. 25A, the arms 54 are pressed or pushed down or extended from a bent configuration (as shown in FIGS. 23 and 24) to a generally straight configuration (as shown in FIG. 25A). As the arms 54 are extended, the sliding portion 56 moves or advances along the outside length of the central tube 51 and away from the fixed portion 52 (the fixed portion 52 may remain in place relative to the central tube 51). The sliding portion 56 may move over or overlap at least a portion of the branches 58, which extend radially outward from the central tube 51 and away from the fixed portion 52. Therefore, as the sliding portion 56 is moved along the length of the tube 51 (and therefore also the branches 58), the branches 58 are forced to conform to the size of the inner diameter of the sliding portion 56. Accordingly, the angle between the branches 58 and the tube 51 decreases and the branches 58 are progressively radially compressed and closed inward over implant 10 (which may still be over the expanding mechanism), thereby radially compressing the implant 10 toward the central axis of the lumen 16, into the pleated configuration 200. In the pleated configuration 200, the implant 10 may have at least one pleat 22 that extends lengthwise along the z-axis and at least partially outwardly from the implant 10, along the x- and y-axes. Depending on the relative sizes of the components, the arms 54 may be fully pressed down to fully advance the sliding portion 56 over the branches 58, increasing or maximizing the radial compression of the implant 10. Further, the branches 58 may align with the flutes 36 in the conical rod 30 for further compression of the implant 10. Accordingly, the implant 10 may be held firmly between the implant compressor 50 and the conical rod 30.

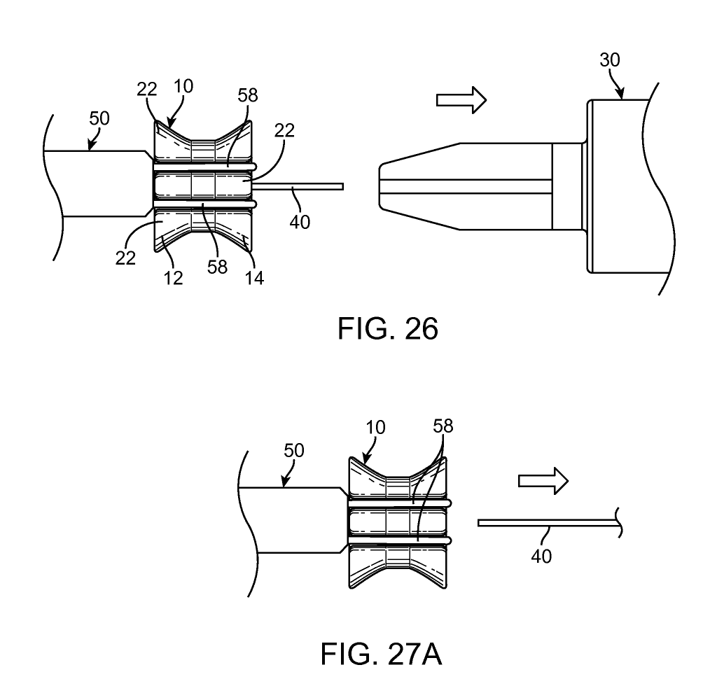

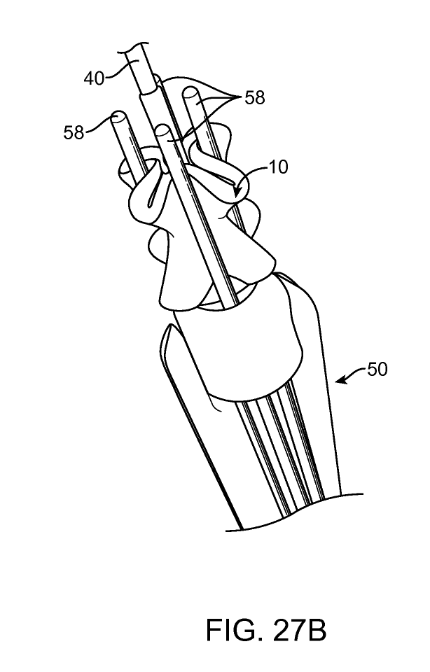

Once the implant 10 is folded into the pleated configuration 200 due to the implant compressor 50 compressing around the implant 10 and the conical rod 30, the conical rod 30 may be removed or withdrawn from within the implant 10 (as shown in FIG. 26). The implant compressor 50 may maintain the compressive force on (and the pleated configuration 200 of) the implant 10 along the central axis as the conical rod 30 is being removed, allowing the implant 10 to be further pleated without the conical rod 30 within the lumen 16. For example, as shown in FIG. 27B, the pleats 22 of the implant 10 may be accentuated into petals or lobes. The centering mandrel 40 may also be removed from within the implant 10 while the implant 10 is held in the pleated configuration 200, as shown in FIGS. 27A and 27B.

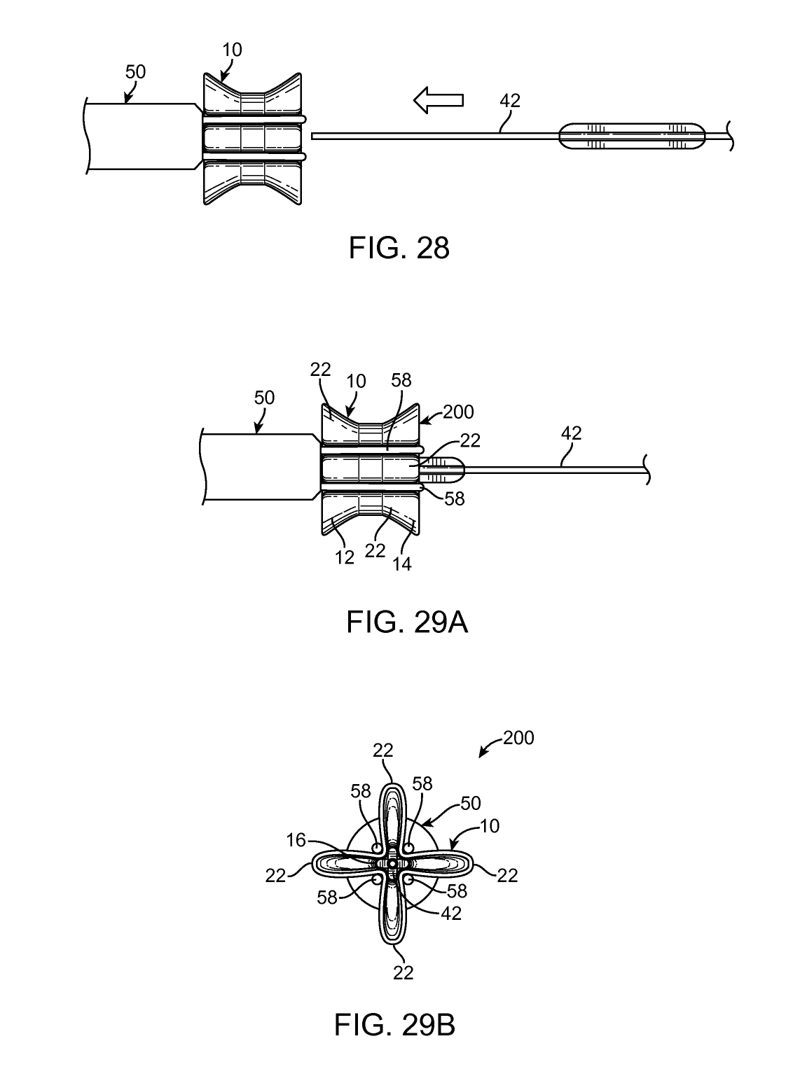

As shown in FIGS. 28 and 29A, the finned mandrel 42 may be inserted or advanced into the lumen 16 of the implant 10 to provide support and/or shape to and maintain the desired configuration of the lumen 16 and the pleats 22 of implant 10. The finned mandrel 42 may additionally extend into the central tube 51 of the implant compressor 50 as well as completely through the implant 10. The finned mandrel 42 may be in any size, shape, or configuration according to the desired folding or pleating configuration and may correspond directly to the pleated configuration 200 of the implant 10. For example, according to one embodiment (as shown in FIGS. 28-29), the finned mandrel 42 may be an elongated mandrel rod or tube with similar properties to that of the centering mandrel 40. The finned mandrel 42 may have a support component made out of a stiff material, such as stainless steel, Teflon or Delrin. The support component may have any shape and may have the same number of radially arrayed fins or arms as the number of pleats 22 of the implant 10. For example, the support component may be cross-shaped or “x”-shaped along the length of the finned mandrel 42 and may have four fins which extend at least partially within four pleats 22 of the implant 10, thereby supporting the pleated configuration 200 or folded shape of the implant 10. Accordingly, as the finned mandrel 42 is inserted into the implant 10, the finned mandrel 42 may be oriented to allow the four fins to extend into the four pleats 22 of the implant 10. However, it is anticipated that the support component may have any number of fins. The finned mandrel 42 may additionally provide a mechanism to align the central longitudinal axis of the various components within the system. The finned mandrel 42 and the centering mandrel 40 may be the same component.

FIG. 29B depicts the accentuated the pleats 22 of the implant 10 (compared to the pleats 22 in FIG. 25B) due to the decreased diameter of the implant 10 (and the removal of the conical rod 30). The branches 58 of the implant compressor 50 may continue to maintain the pleated configuration 200 of the implant 10. As shown, the fins of the finned mandrel 42 are within the implant 10 and directly fit within the folds or the pleats 22. The folding pattern of the implant 10 may be adjusted at various points during the loading and delivery of the implant 10 to ensure that the implant 10 will properly deploy.

Once the finned mandrel 42 is inserted into the lumen 16, the implant 10 may be transferred into the loading tube or tool 74. The loading tool 74 may serve as a conduit for the implant 10 as the implant 10 is moved into the delivery system. The loading tool 74 may be used to accept the implant 10 in the pleated configuration 200 and may progressively limit the diameter of the implant 10 into the compressed configuration 210. Further, the loading tool 74 may position and hold the capsule 110 of the delivery device 100 in place while the implant 10 is being loaded into the implant positioner 120 and, subsequently, into the capsule 110. The loading tool 74 may be at least partially transparent.

The loading tool 74 may be multiple individual, separate, and/or detachable components that fit, snap, or lock together (as shown in FIGS. 16C-16E), such that each component of the loading tool 74 shares a central horizontal axis and the implant 10 may smoothly move through the central lumen of the loading tool 74 (the implant 10 may move right to left in FIGS. 16D and 16E). The loading tool components may include, but are not limited to, the tapered tube 60, the sheath holder 70, and the capsule loading section 80. The tapered tube 60 may have a fluted funnel section to accept the implant 10 in the pleated configuration 200. The funneled section may progressively compress the formed pleats towards the implant 10 and into the compressed configuration 210. The sheath holder 70 may be used to accept the folded and compressed implant 10 and to load the implant 10 into the sheath 90. The sheath holder 70 may be reversible, allowing the implant 10 to be inserted into and pushed out of the sheath holder 70 in either direction or orientation. According to another embodiment, the sheath holder 70 may house or contain the sheath 90 into which the implant 10 may be packed and constrained. The sheath 90 may also allow the implant 10 to be flipped during the loading process. The capsule loading section 80 may be used to load the implant 10 into the delivery device 100 to be delivered into a patient. More specifically, the capsule loading section 80 may align with the capsule 110 (and its integral implant positioner 120) of the delivery device 100 to allow the implant 10 to be pushed at least partially into the implant positioner 120 and the capsule 110.

The multiple separate components of the loading tool 74 may provide a greater flexibility during use and for complex machine operations to be performed during fabrication. However, according to another embodiment as shown in FIG. 18, a loading tool 174 may be used to transfer the implant 10 into the delivery device 100. The loading tool 174 may include various sections, such as a capsule loading section 180, a sheath holder 170, and a tapered tube 160, that are continuously connected. The loading tool 174 may be a single component with a series of features attached or fixed together and incorporated into its inner lumen. For example, the implant 10 may be inserted into the entry end (e.g. the tapered tube 160) of the loading tool 174 and pushed through the loading tool 174 toward the exit end (e.g. the capsule loading section 180). The sheath 90 may optionally be integrated into the assembly. According to another embodiment, the loading tool 174 may only include the tapered tube 160 and the capsule loading section 180.

As shown in FIG. 18, the capsule loading section 180 may have an angled portion for the implant positioner 120 to radially expand into. However, as shown in FIGS. 16D-16E, the capsule loading section 80 may be generally straight or flat. It is anticipated that the characteristics and embodiments of either of the loading tools 74 or 174 may be used.

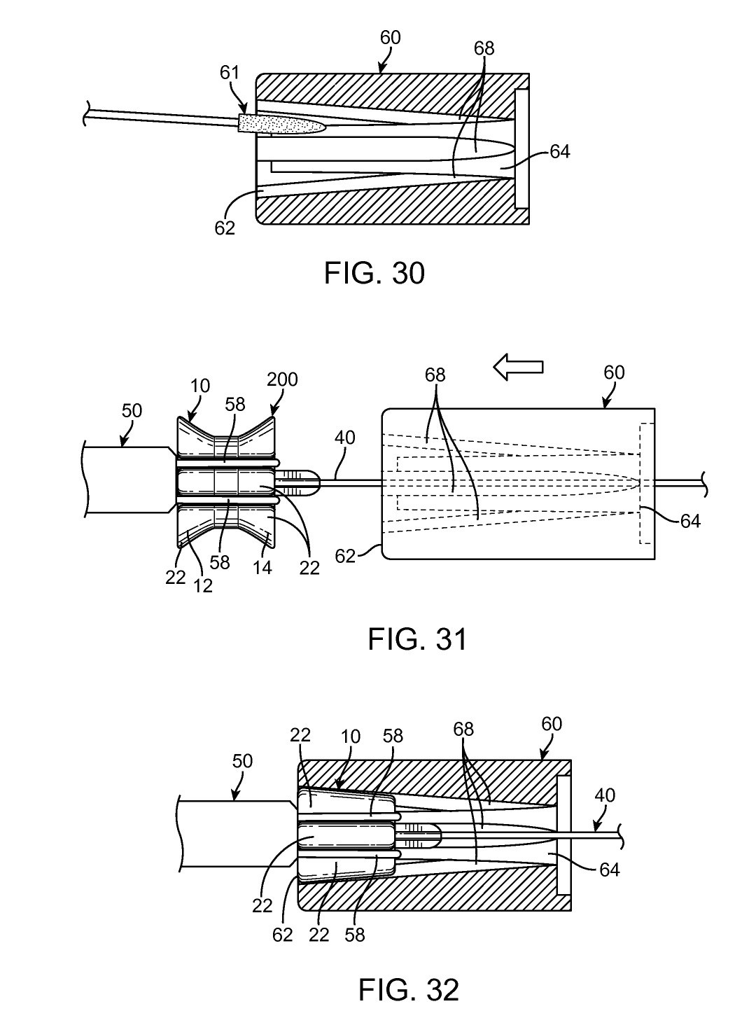

According to one embodiment, FIG. 30 depicts the inside of the tapered tube 60 being lubricated with a lubrication device 61 in preparation for the implant to be loaded into the loading tool 74. The tapered tube 60 may include an inner tapered tube with flutes 68. The flutes 68 run at a slight angle along the longitudinal axis of the tapered tube 60, such that the tapered tube 60 has an expanded end 62 with a relatively larger diameter and a compressed end 64 with a relatively smaller diameter. The flutes 68 may be configured, sized, and oriented according to the desired configuration of the folded or compressed implant 10.

As shown in the embodiment of FIGS. 31 and 32, the implant 10 (being held between the implant compressor 50 and the finned mandrel 42) is advanced or slide into the widest end of the tapered tube 60 to radially compress the implant 10 into the compressed configuration 210. The implant 10 may be oriented or aligned such that the pleats 22 directly line up with and fit within the flutes 68 to allow the implant 10 to be orderly compressed. The implant compressor 50 may maintain the pleated configuration 200 of the implant 10 as the implant 10 is being advanced into the tapered tube 60. The implant 10, the implant compressor 50, and the finned mandrel 42 are inserted into the expanded end 62 of the tapered tube 60 and toward the compressed end 64 of the tapered tool. As the implant 10 is advanced into the tapered tube 60 toward the compressed end 64, the branches 58 may maintain the pleats 22 of the implant 10 and the implant 10 may be further compressed toward the compressed configuration 210. The tapered tube 60 may optionally be transparent, clear, or translucent to allow the configuration and orientation of the implant 10 to be shown.

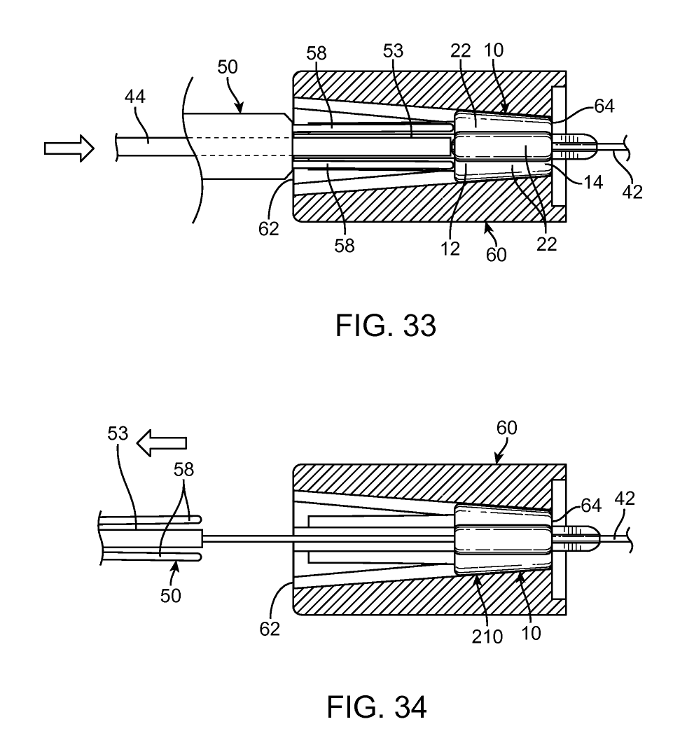

As shown in FIGS. 32 and 33, the implant 10, with the implant compressor 50 and the finned mandrel 42, may be advanced along the longitudinal axis of the tapered tube 60. Due to the tapered inside of the tapered tube 60, as the implant 10 is advanced from the expanded end 62 to the compressed end 64, the implant 10 is radially and tightly compressed, the pleats 22 are forced down toward the lumen 16 of the implant 10, and the outside diameter of the implant 10 is reduced.

Once the implant 10 is completely within the tapered tube 60, the implant compressor 50 may be removed as the implant 10 is pushed further along the tapered tube 60. The implant 10 may be advanced through the tapered tube 60 through a variety of means. According to one embodiment as shown in FIG. 33, in order to remove the implant compressor 50 from the implant 10, the plunger 59 may be advanced, which may advance an inner rod or tube (within the central tube 51) to move the implant ejector 53, pushing the implant 10 further into the loading tool 74. As the implant ejector 53 pushes the implant 10, the implant 10 is released from the branches 58, and the flutes 68 of the tapered tube 60 maintain the compressed configuration 210 of the implant 10. Accordingly, the implant compressor 50 is then removed from the tapered tube 60, as shown in FIG. 34. Additionally, the end of the implant compressor 50 may exert a pressure or force on the compressed implant 10 to further move the implant 10 into the loading tool 74. As the implant 10 is moved along the tapered tube 60, the tapered configuration of the tapered tube 60 further compresses the implant 10, preparing the implant 10 to moved further along the loading tool 74. The finned mandrel 42 may remain in place to maintain the configuration and folding of the implant 10, as well as align components within the system. According to another embodiment, the finned mandrel 42 may be removed.

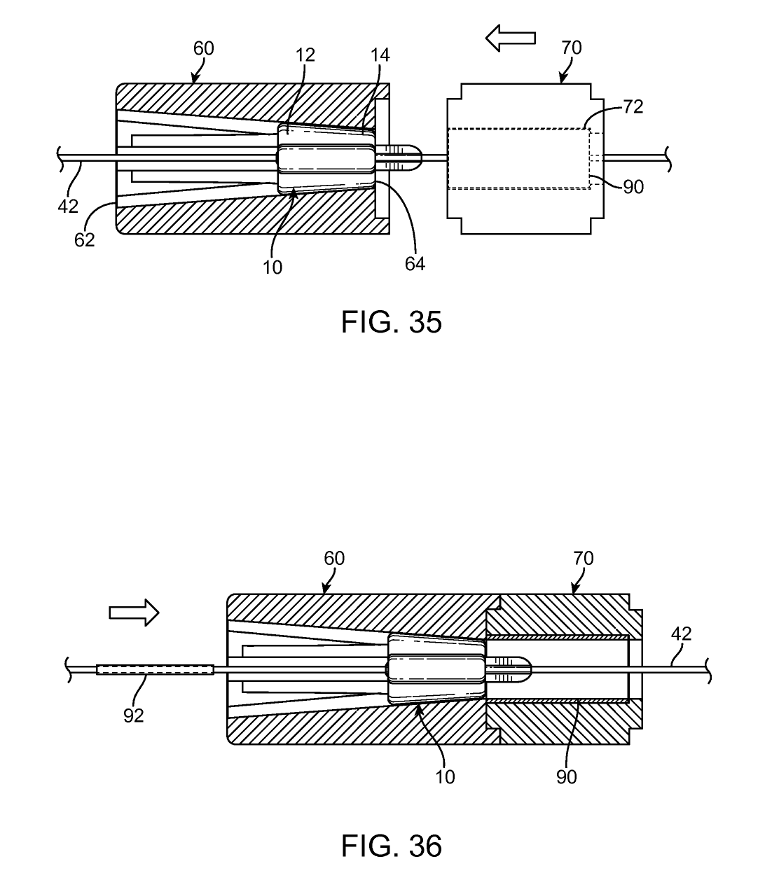

As shown in FIG. 35, in one embodiment, the sheath holder 70 is removably attachable or engagable to the compressed end 64 of the tapered tube 60. The sheath holder 70 and the tapered tube 60 have complementary ends, such that the ends may fit with each other. Alternatively, the sheath holder 70 and the tapered tube 60 may be permanently attached or one unit.

The sheath 90 may be located or contained within a central lumen of the sheath holder 70 and may be held in place on one side by a lip 72, thereby preventing the sheath 90 from sliding out of sheath holder 70 as the implant 10 is being advanced into the sheath holder 70. Alternatively, the sheath holder 70 may not include the sheath 90 and the inner diameter of the sheath holder 70 may be equal to the inner diameter of the compressed end 64.

As shown in FIG. 36, the tube 92 may be inserted into the lumen 16 of the implant 10 in order to provide an open, inner passageway or shaft within the implant 10, particularly while the implant 10 is in the compressed configuration 210. This may allow a balloon catheter, for example, to move through the inner lumen of the implant 10 while the implant 10 is in the compressed configuration 210. The tube 92 may be approximately the same length as the implant 10 in the compressed configuration 210, such that the tube 92 does not stick out beyond the edges of the implant 10. The tube 92 may be moved along the finned mandrel 42 to accurately be inserted into the lumen 16. The tube 92 may be inserted into the lumen 16 through either end of the implant 10 and may be slide over the finned mandrel 42.

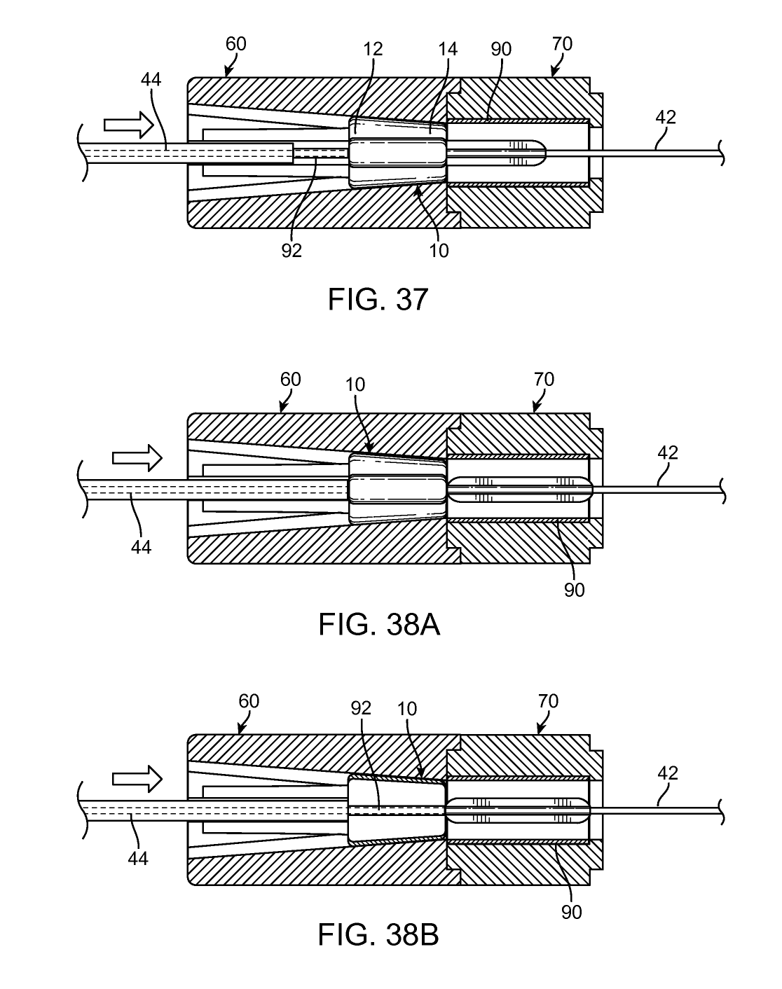

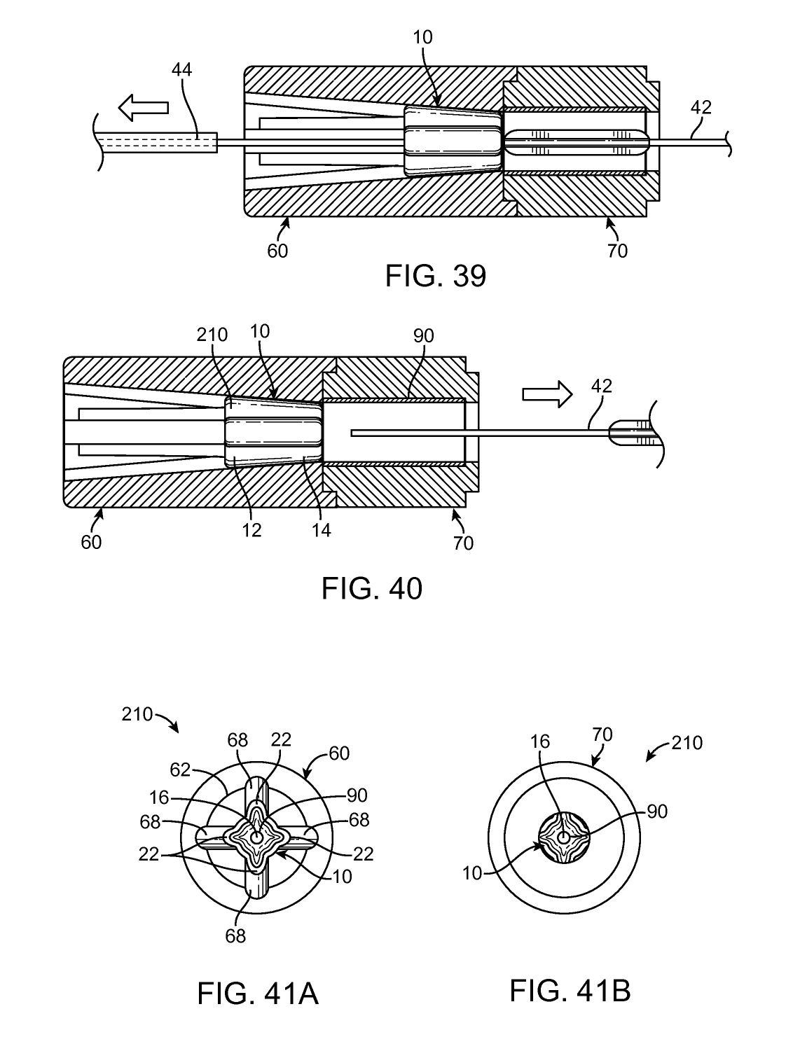

As shown in FIGS. 27-38, to advance the tube 92 into the lumen 16, a pushing device, such as the rod 44 or 46 may be used. The rod 44 may be advanced around the finned mandrel 42 to be properly aligned with the lumen 16 and the tube 92. As the rod 44 pushes the tube 92 into the lumen 16, the tube 92 forces the fins of the finned mandrel 42 out from the lumen 16, as shown in FIG. 38B. Once the tube 92 has been properly placed, the rod 44 and the finned mandrel 42 may be removed from the tapered tube 60 and the sheath holder 70, as shown in FIGS. 39 and 40, while the tube 92 remains in place within the implant 10.

As shown in FIGS. 41A and 41B, the implant 10 may be compressed within the tapered tube 60 in the folded and compressed configuration 210. The tube 92 provides an open passageway between either side of the implant 10 within the lumen 16 of the implant 10. As shown in FIG. 41A with a view through the expanded end 62 of the tapered tube 60, the implant pleats 22 may line up with the flutes 68 within the tapered tube. The opposite side of the sheath holder 70, as shown in FIG. 41B, may be circular and form a passageway for components such as the centering mandrel 40 or the finned mandrel 42.

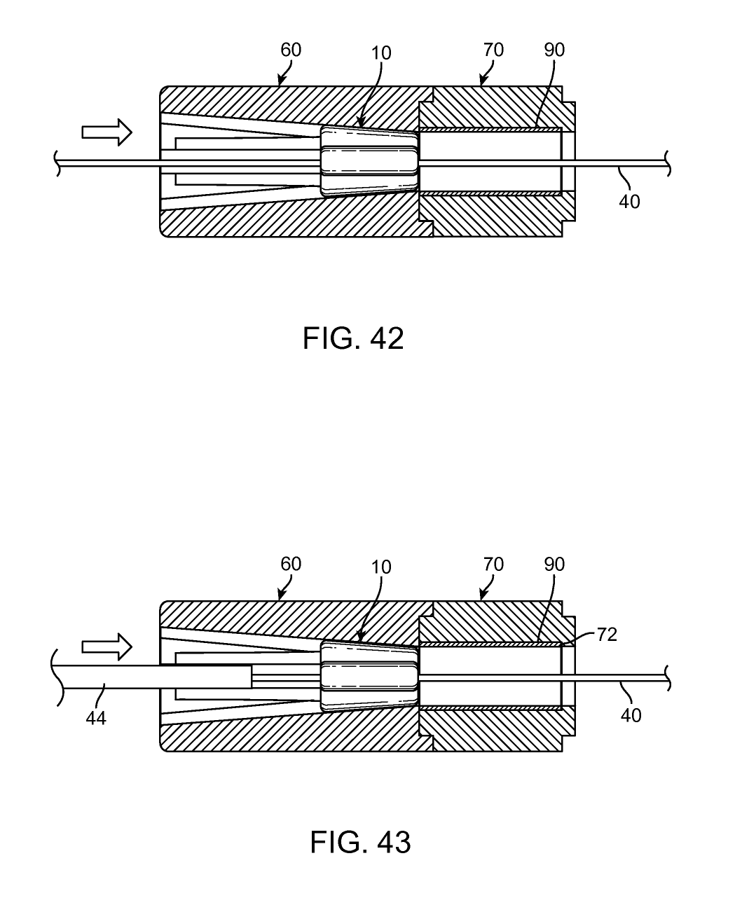

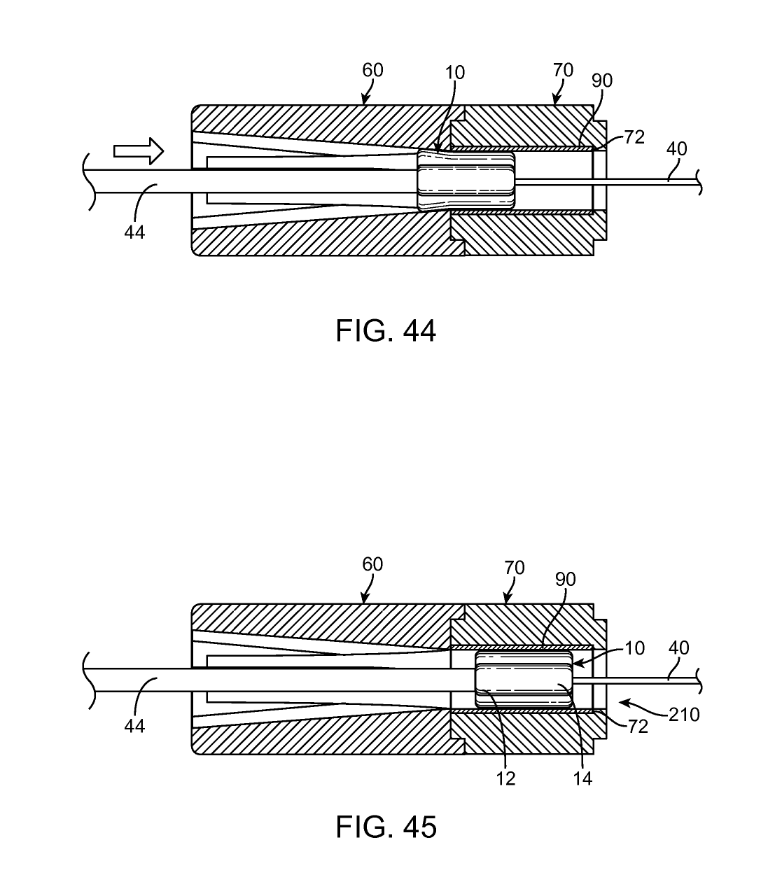

As shown in FIG. 42, in one embodiment, the centering mandrel 40 is inserted into or through the tube 92 and through both the tapered tube 60 and the sheath holder 70. The rod 44 or 46 is advanced over the centering mandrel 40, as shown in FIG. 43, in order to push or advance the implant 10 from the tapered tube 60 to the sheath holder 70, as shown in FIGS. 44 and 45. At the same time, the implant 10 may also be inserted or advanced into the sheath 90 within the sheath holder 70. As described previously, the sheath 90 may be held in place due to the lip 72, which may be located around the circumference of one side of the sheath holder 70. As the implant 10 is moved from the tapered tube 60 to the sheath holder 70, the implant 10 may be slightly more radially compressed or compacted along the central axis into the compressed configuration 210 (while the pleated configuration 200 of the implant 10 is maintained). The additional compression may further decrease the diameter of the implant 10 such that the diameter of the implant 10 is approximately equal to or less than an inner diameter of a loading mechanism or device. The sheath 90 may further maintain or secure the compressed configuration 210 of the implant 10.

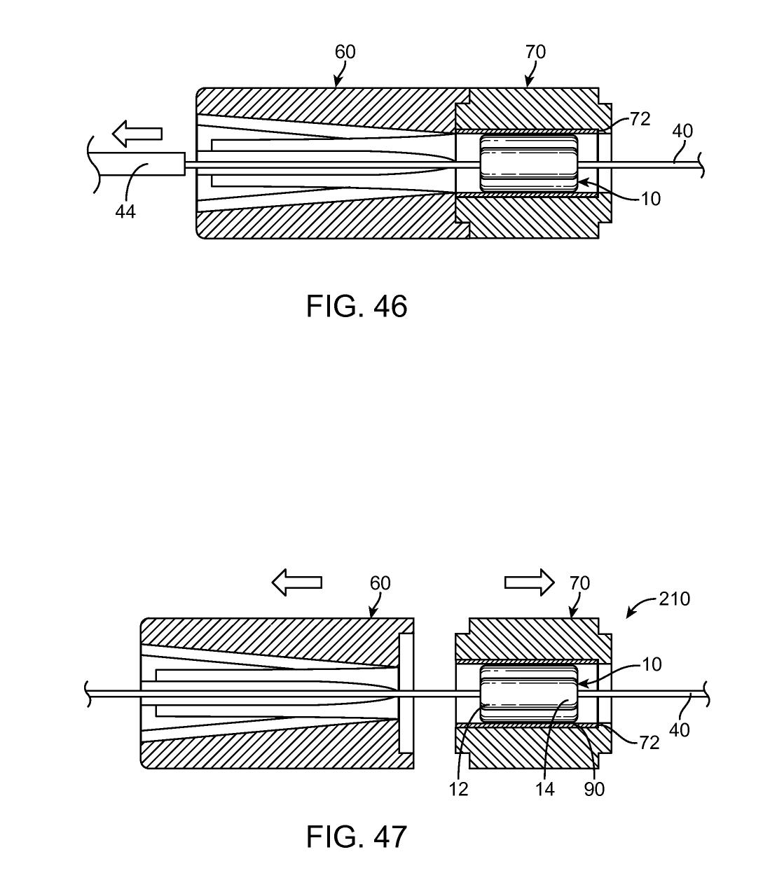

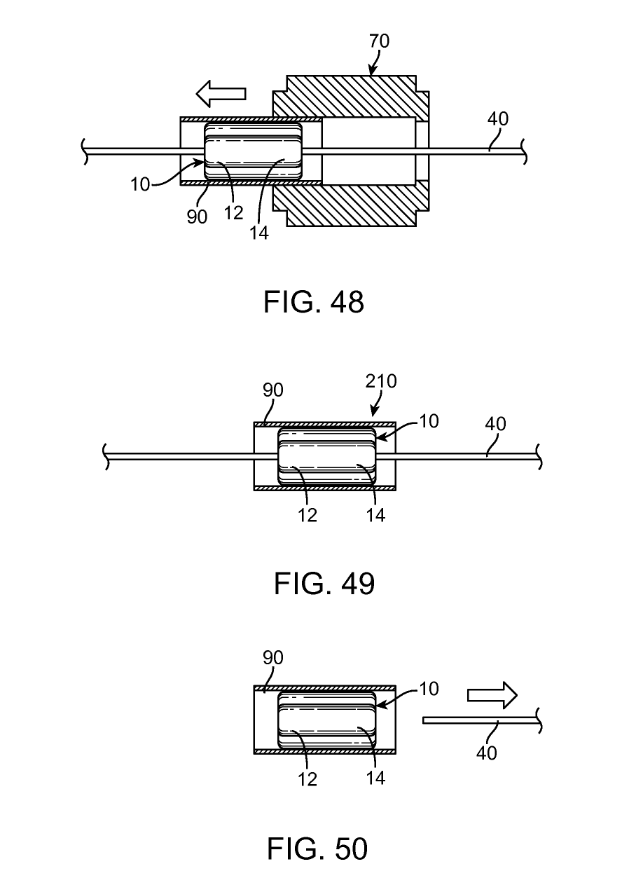

Subsequently, after the implant 10 has been moved into the sheath holder 70 (and/or the sheath 90), the rod 44 is removed from the tapered tube 60, as shown in FIG. 46. The tapered tube 60 and the sheath holder 70 may be detached or disconnected, as shown in FIG. 47, and the implant 10 (within the sheath 90 and with the centering mandrel 40) is removed from the sheath holder 70, as shown in FIG. 48.

FIG. 49 depicts the implant 10 within the sheath 90 in the compressed configuration 210. The implant 10 may be flipped relative to the sheath holder 70 and inserted into the delivery device or the centering mandrel 40 may be removed from the tube 92 in the lumen 16 of the implant 10, as shown in FIG. 50. The implant 10 may be, for example, marketed, shipped, and sold with the sheath 90 and the tube 92 to be later advanced into a delivery device. The sheath 90 may additionally be lubricated with, for example, a water-soluble lubrication, silicone, and/or silicone coated with a low friction material (e.g. parylene). Before placing into the body of the patient, the implant 10 (and the sheath 90) may be sterilized through a variety of techniques, including but not limited to ETO or EB sterilization.

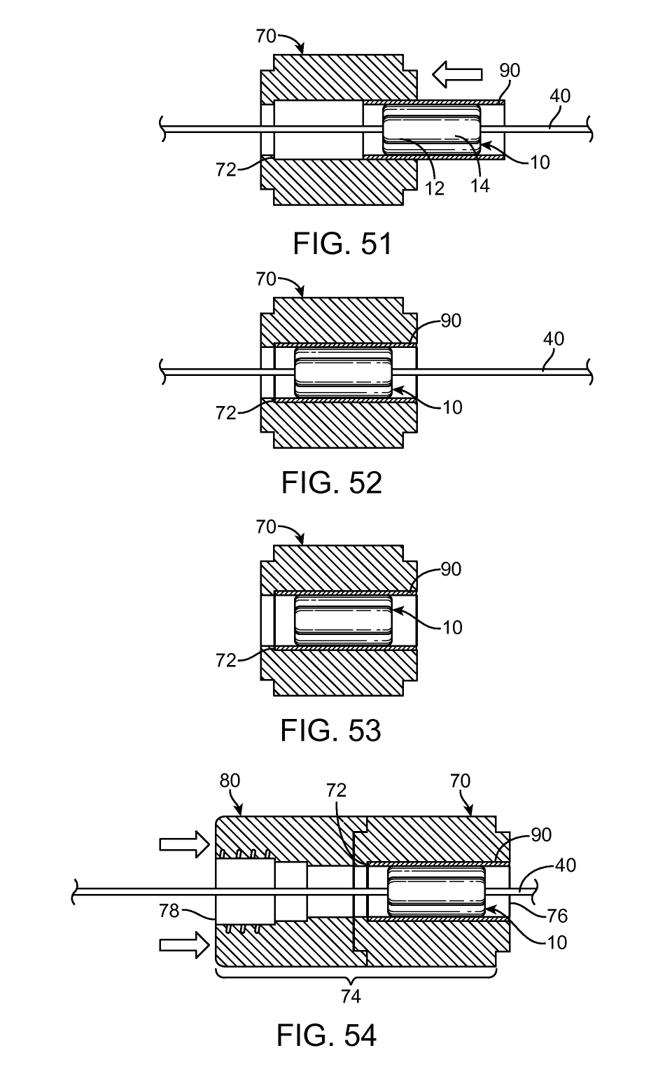

Referring now to FIG. 51, the centering mandrel 40 may be reinserted into the tube 92 and the implant 10 may be placed back into the sheath holder 70. However, the sheath holder 70 and the implant 10 may be flipped relative to each other, such that the flange 12 is proximal to the lip 72, instead of having the flange 14 proximal to the lip 72 as shown previously (e.g. FIG. 45). Flipping the implant 10 and the sheath holder 70 relative to one another allow for a particular side to be loaded into the delivery device first according to which the flange 12 or 14 is desired to be deployed in particular cavities within the body. The implant 10 may be inserted into the sheath holder 70 with (as shown in FIG. 52) or without (as shown in FIG. 53) the centering mandrel 40.

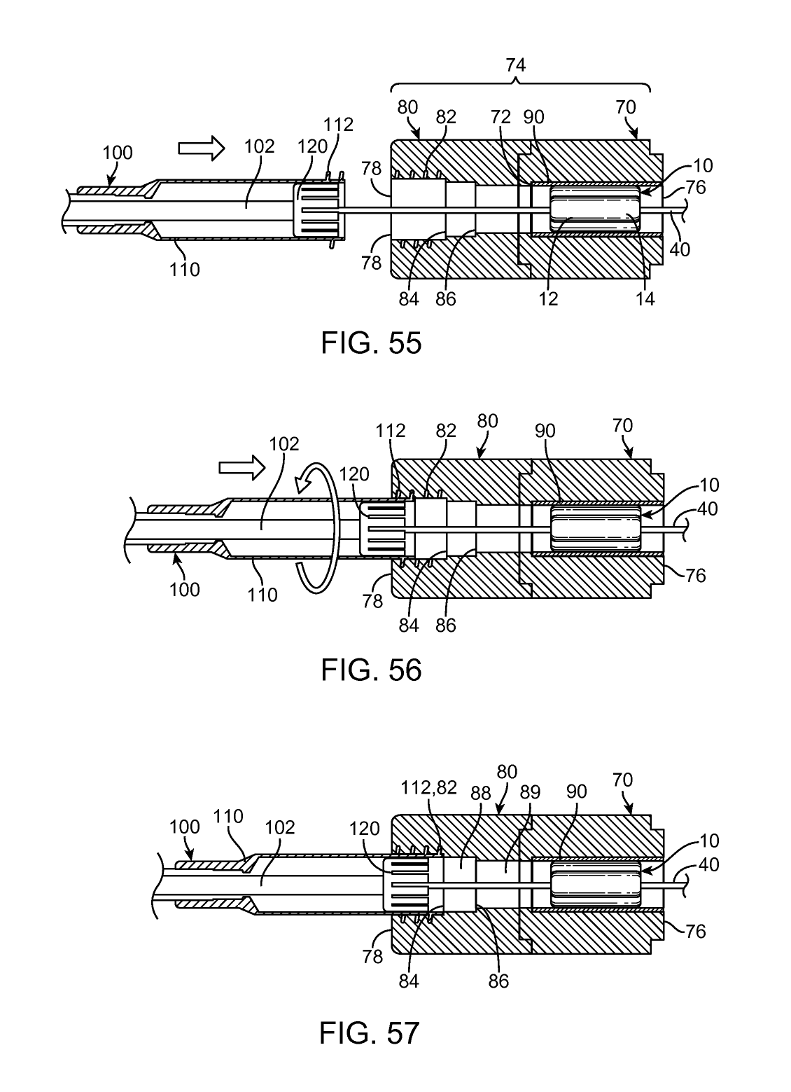

The capsule loading section 80 allows the compressed implant 10 to be loaded into the delivery device 100 to allow for proper deployment. As shown in FIG. 54, the capsule loading section 80 may be attached to the sheath holder 70, proximal to the lip 72. Alternatively, the capsule loading section 80 and the sheath holder 70 may be one unit, such as the loading tool 74 (the loading tool 74 may alternatively or additionally include the tapered tube 60). The implant 10, in the compressed configuration 210, may be advanced or inserted through a first end 76 of the loading tool 74, while the delivery device 100 may be advanced through a second end 78 of the loading tool 74, as shown in FIG. 55.

As shown in FIGS. 55-57, the delivery device 100 may attach or mate with the capsule loading section 80 in order to align the lumen 16 to the central lumen of the delivery device 100. The delivery device 100 may be aligned with the loading tool 74 by sliding over the centering mandrel 40 that extends through the compressed implant 10 within the loading tool 74. According to one embodiment, the capsule 110, which may be located at the distal end of the delivery device 100, may include external threads 112 on the distal end of the capsule. The threads 112 correspond to and mate with internal threads 82 within the inside of the capsule loading section 80, proximal to the second end 78 of the loading tool 74. Therefore, as the delivery device 100 approaches the capsule loading section 80, the delivery device 100 (or the capsule 110) may be rotated in order to engaged the threads of the outer surface of the capsule 110 and the inner surface or lumen of the loading tool 74. This may allow the capsule 110 to screw or insert at least partially into the second end 78 of the loading tool 74 or the capsule loading section 80, as shown in FIG. 56. However, it is anticipated that the capsule loading section 80 may not have threads and may attach with the capsule through other mechanisms, such as a frictional force or snaps.

The capsule 110 is advanced within the capsule loading section 80 until coming into contact with a first step 84, as shown in FIG. 57. As shown, the inner diameter of the capsule 110 may approximately match the inner diameter of an implant positioner section 88. In order to properly align the implant 10 and the delivery device 100, the centering mandrel 40 may be at least partially extended or inserted into the central lumen of the delivery device 100 and through the lumen 16 of the implant 10.

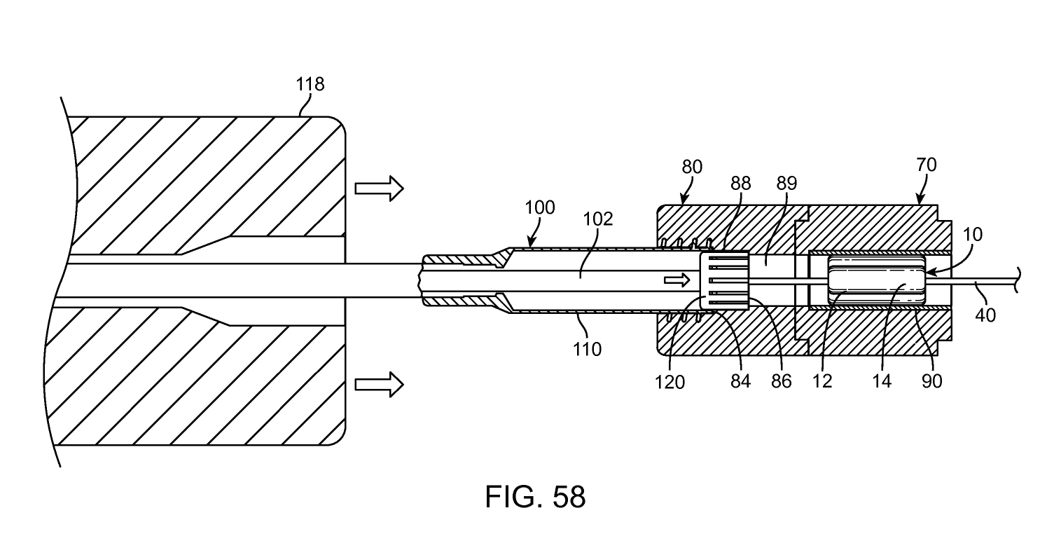

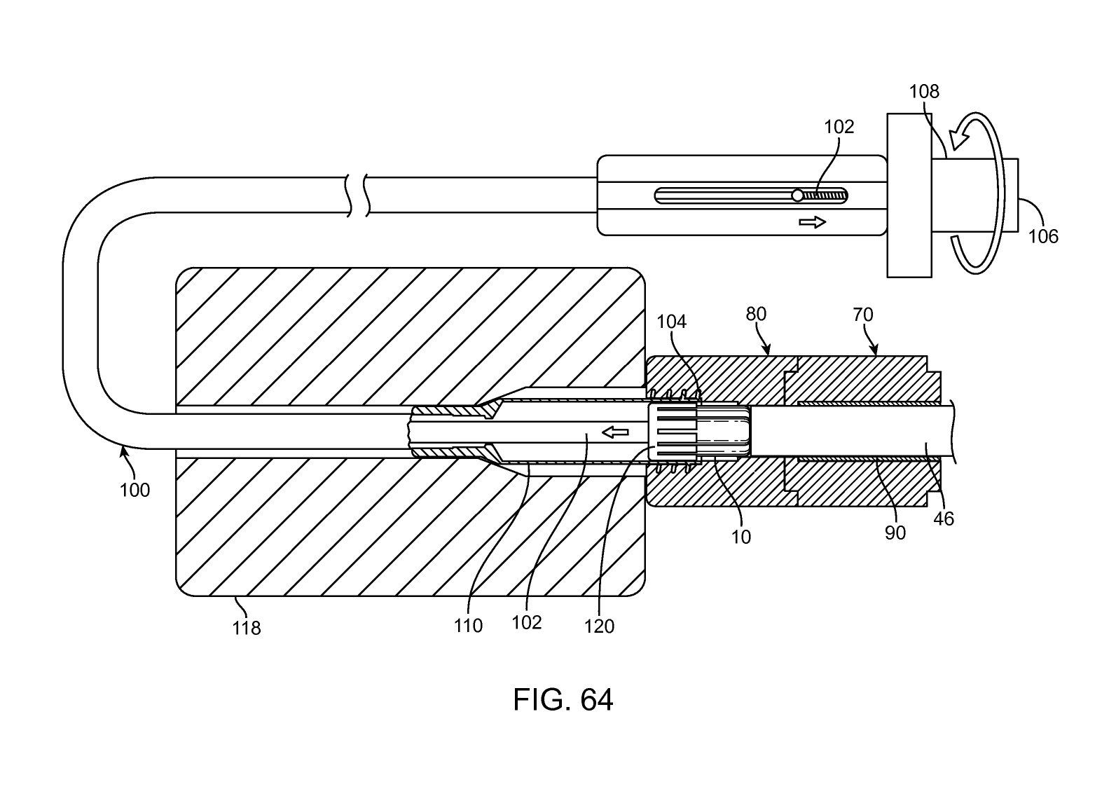

As shown in FIG. 58, in one embodiment, the implant positioner 120 advances or extends at least partially beyond the distal end of the capsule 110 into the implant positioner section 88 to a second step 86. The implant positioner 120 may be directly connected to a central coil, as shown as, but not limited to, a pusher coil 102. The pusher coil 102 may be located within a central lumen of the delivery device 100 and may extend from a proximal end 106 to a distal end 104 of the delivery device 100 (as shown in FIG. 64). The pusher coil 102 may be extended in order to advance the implant positioner 120 out of the capsule 110 and toward the compressed implant 10. According to one embodiment, the implant positioner 120 may at least partially radially expand within the loading tool 74 as the implant positioner 120 is advanced toward the implant 10. The inner diameter of the implant positioner 120 may be approximately the same as the inner diameter of an implant advancement section 89, such that the compressed implant 10 has a smooth transition into the delivery device 100. The implant positioner 120 may include flexible fingers allow the implant 10 to be deployed out of the capsule 110.

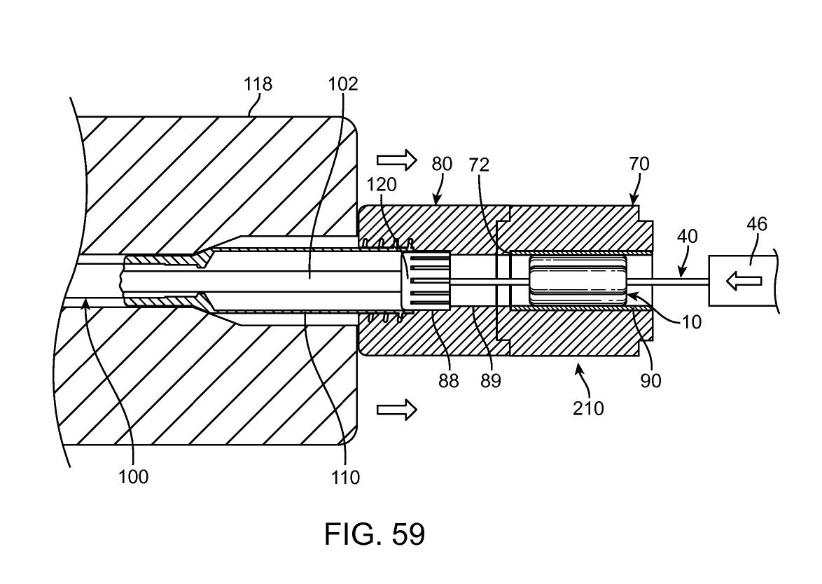

As further shown in FIGS. 58 and 59, the loading grip 118 may be slide over a portion of the delivery device 100 and advanced until it contacts the capsule 110. The loading grip 118 may be used to hold the capsule 110 with respect to the capsule loading section 80 and may improve the user's grip and leverage on the delivery device 100 through the loading process. For example, the loading grip 118 may have a hollow shaft with a longitudinal slit to insert and secure a portion of the capsule 110.

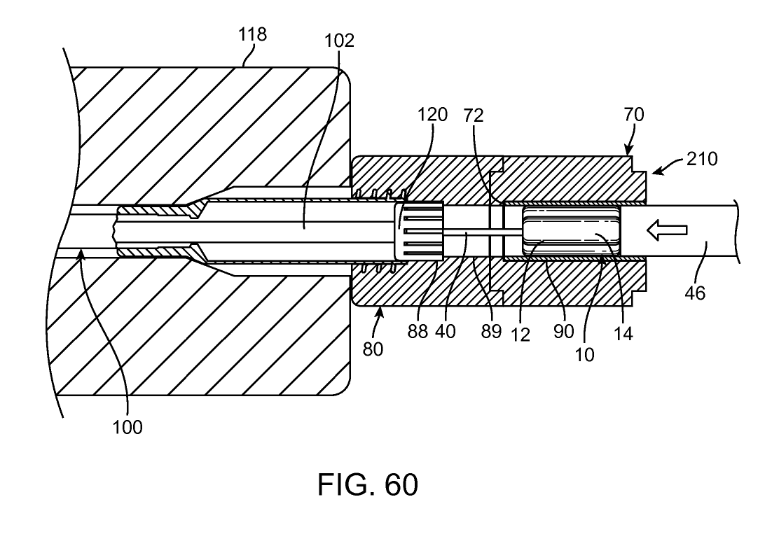

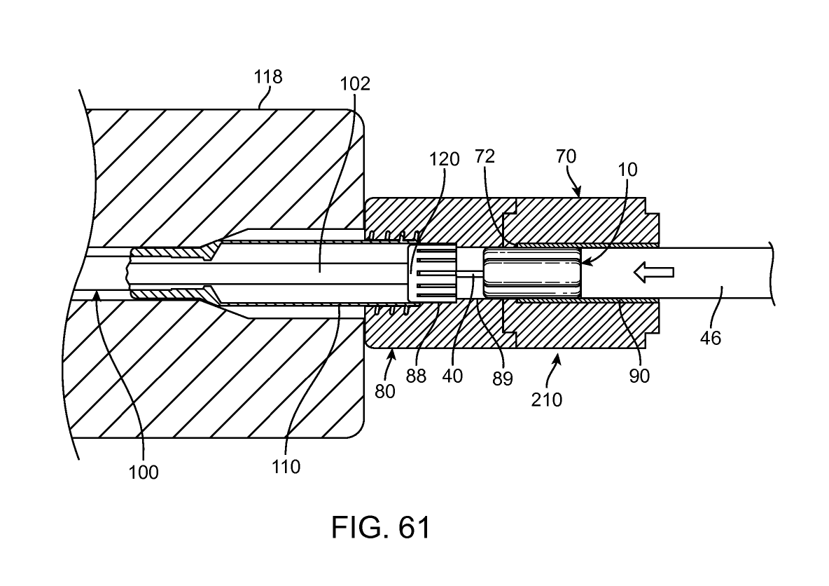

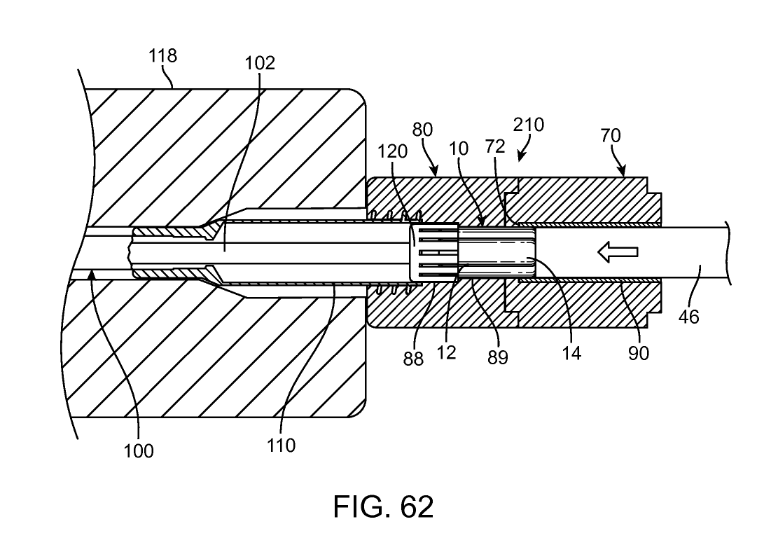

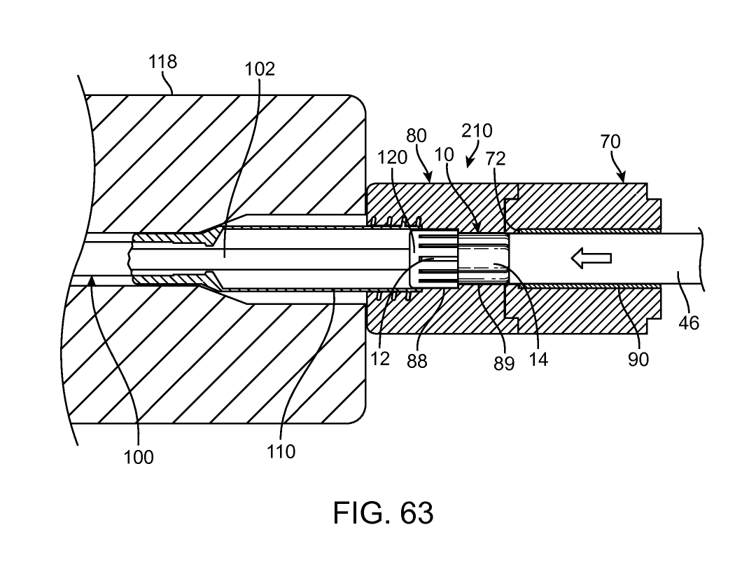

As shown in FIG. 59-63, a pushing device, such as the rod 46, may be used to advance or push the implant 10 from the sheath holder 70 to the implant advancement section 89 within the capsule loading section 80 and further into the implant positioner 120. The outer diameter of the implant 10 may be approximately the same as the inner diameter of the capsule 110. At least a portion of the implant 10 may be advanced partially into the implant positioner 120. For example, the flange 12 may be first inserted or advanced into the implant positioner 120 and the flange 14 may remain outside of or not be secured by the implant positioner 120. The implant positioner 120 may be sized in order to only hold one of the flanges 12 or 14. Accordingly, the flange 14 may not be contained or constrained by the implant positioner 120. This allows the flange 14 to deploy within the patient separately from when the flange 12 deploys within the patient. The rod 46 may be inserted over the centering mandrel 40 to ensure proper alignment with the implant 10. Due to the lip 72, the sheath 90 may stay within the sheath holder 70 and separate from the implant 10 as the implant 10 is pushed into the capsule loading section 80 (in order to advance or place the flange 12 into the implant positioner 120). The outside diameter of the implant 10 in the compressed configuration 210 (as well as the inside diameter of the sheath 90) is approximately equal to the inside diameter of the implant advancement section 89, providing a smooth transition into the implant positioner 120.

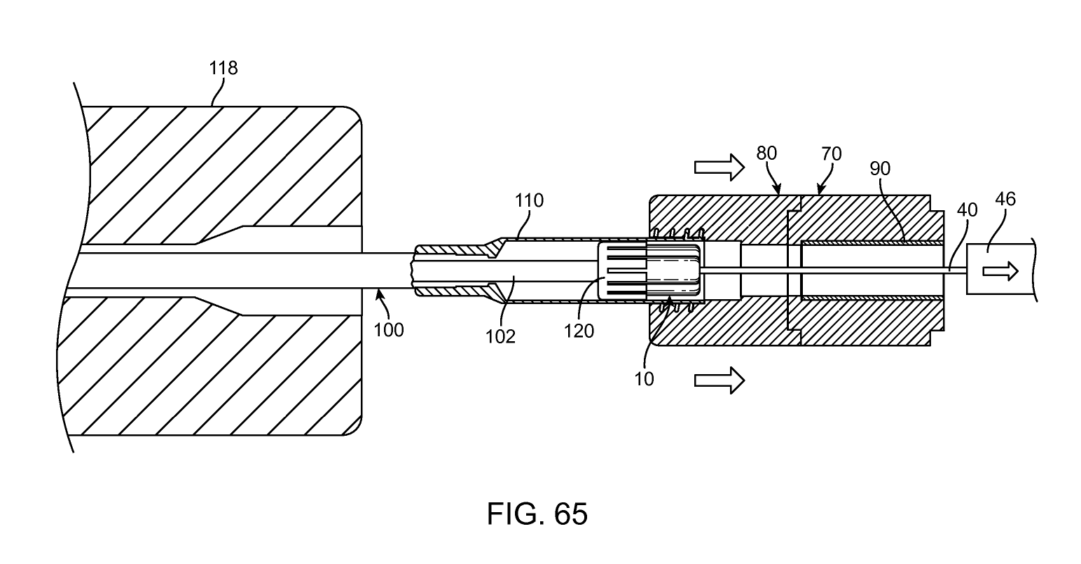

As shown in FIGS. 64 and 65, the implant positioner 120, with the implant 10, is then at least partially retracted back into or within the capsule 110. The rod 44 or 46 may also be used to help push the implant 10 into the implant positioner 120 until both the implant positioner 120 and the implant 10 are at least partially within the capsule 110. The capsule 110 may first cover or encapsulate at least a portion of the implant positioner 120 (containing the flange 12) and then subsequently cover or encapsulate the rest of the implant 10 (e.g. the flange 14). According to one embodiment, due a differential in the diameters of the implant positioner 120 and the capsule 110, the implant positioner 120 may at least partially collapse or compress around the flange 12 as the implant positioner 120 is being retracted back into the capsule 110.

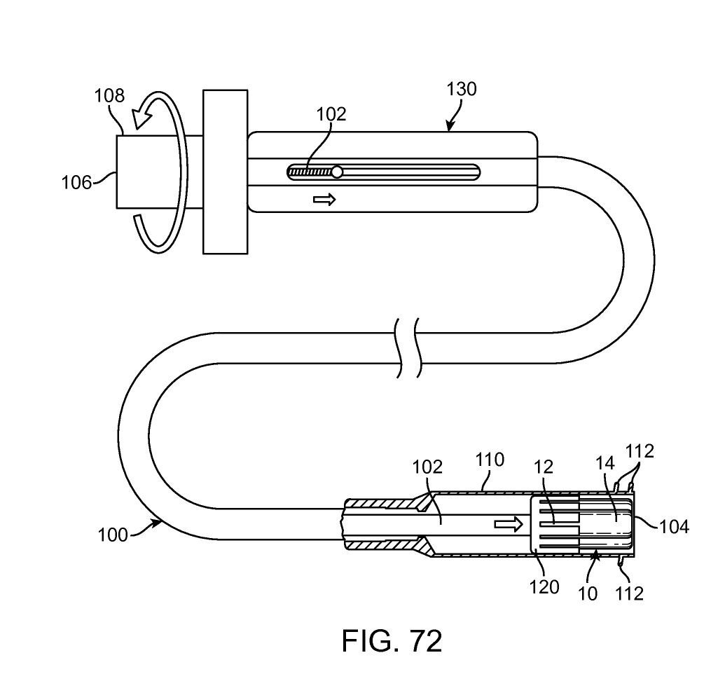

In order to retract the implant positioner 120 back into the capsule 110, the pusher coil 102 is retracted within the delivery device 100 from the distal end 104 to the proximal end 106 of the delivery device 100. For example, a control device or handle 130, 630, or 730 of the delivery device 100 may have a rotating knob, control or device 108 or 708 to control the length of the central lumen of the delivery device 100 and, thereby, the positioning of the pusher coil 102. The rotating device 108 be rotated in order to reduce the length of the central lumen of the delivery device 100 (e.g. the pusher coil 102), drawing or retracting the pusher coil 102 and the implant positioner 120 with the implant 10, toward the proximal end 106 and into the capsule 110. Moving the implant positioner 120 may be augmented by applying pressure with the rod 44 or 46. The rotating device 108 may also be rotated in the opposite direction to extend the implant positioner 120 from the capsule 110. According to one embodiment as shown in FIG. 72, the rotating device 108 may be located on a proximal end of the delivery device 100 (e.g. on the opposite end from the capsule 110 and the implant positioner 120).

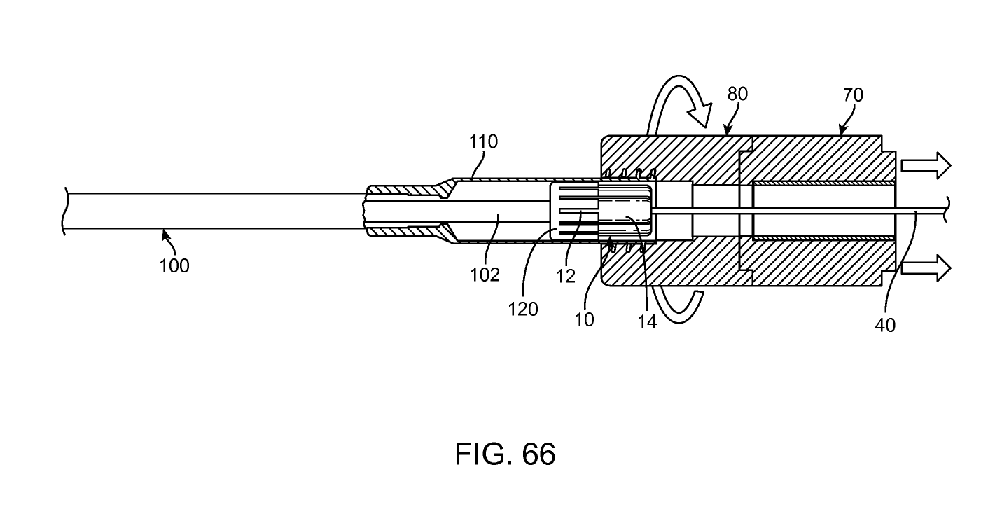

FIG. 65 depicts the implant positioner 120 and the implant 10 within the capsule 110. The loading grip 118 and the rod 46 may be removed from the system. FIG. 66 depicts the capsule loading section 80 and the sheath holder 70 being removed, unscrewed, or uncoupled from the delivery device 100. In order to detach the capsule loading section 80 from the capsule 110, the capsule loading section 80 must be unscrewed, as shown.

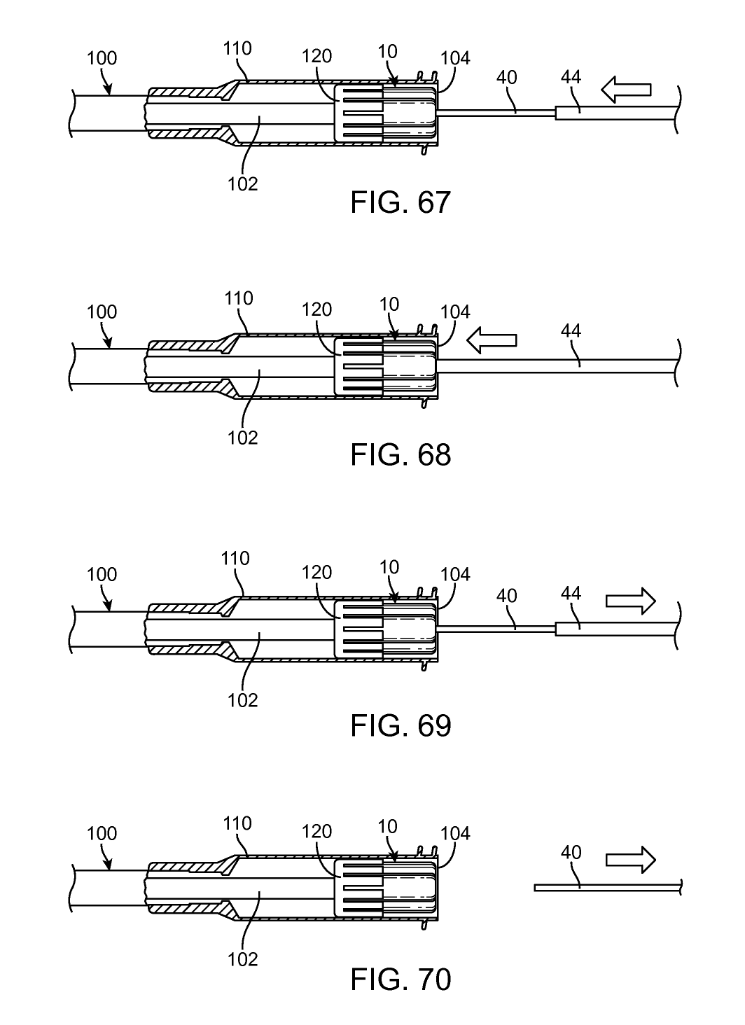

FIGS. 67-70 depict the rod 44 moving or repositioning the implant 10 and the tube 92 fully into the capsule 110. For example, the rod 44 may be used to push in or advance the implant 10 and/or a portion of the tube 92 (within the lumen 16 of the implant 10) that may be extending beyond the distal end of the delivery device 100, as shown in FIGS. 67 and 68, into the capsule 110. The rod 44 moves along the centering mandrel 40 in order to properly align with the lumen 16. Subsequently, the rod 44 and the centering mandrel 40 may be removed from implant 10 (as shown in FIGS. 69 and 70) and the implant 10 may be deployed from the delivery device 100.

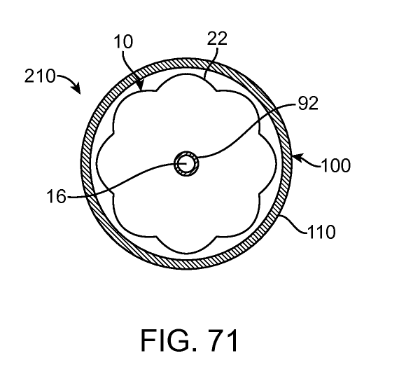

FIG. 71 depicts the implant 10 in the compressed configuration 210 within the capsule 110 of the delivery device 100. The pleats 22 are disposed around the outer circumference of the implant 10 and may include the tube 92 within the lumen 16 of the implant 10.

Although certain embodiments of the loading device are referred to, it is anticipated that a variety of different embodiments of the loading devices may be interchangeable and used to fold and load the implant into the delivery device.

### Deployment Devices and Procedures

Referring generally to the figures, described herein is a delivery system and device 100 and 700 that may be used to endoscopically deliver, position, place, and deploy an implantable device at a desired location within the body of the patient. The methods and apparatus described herein may be used with a variety of different medical procedures and surgeries and a variety of different implantable devices, which may be used to connect two bodily lumens. For example, the delivery device 100 or 700 may be used to deliver and deploy the implant 10 between the gallbladder and the duodenum. According to one embodiment, the delivery device 100 or 700 may provide wire-guided delivery. As shown in FIGS. 72-76, a delivery device 100 and 700 may have a variety of different components and configurations, which may be used interchangeably.

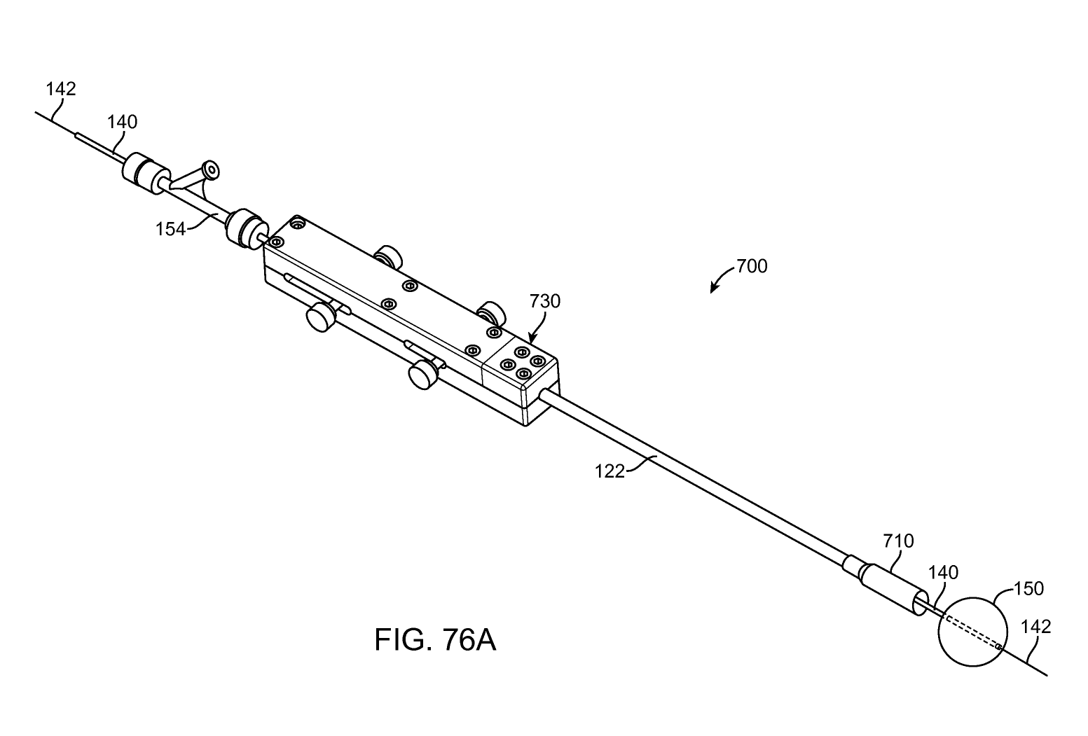

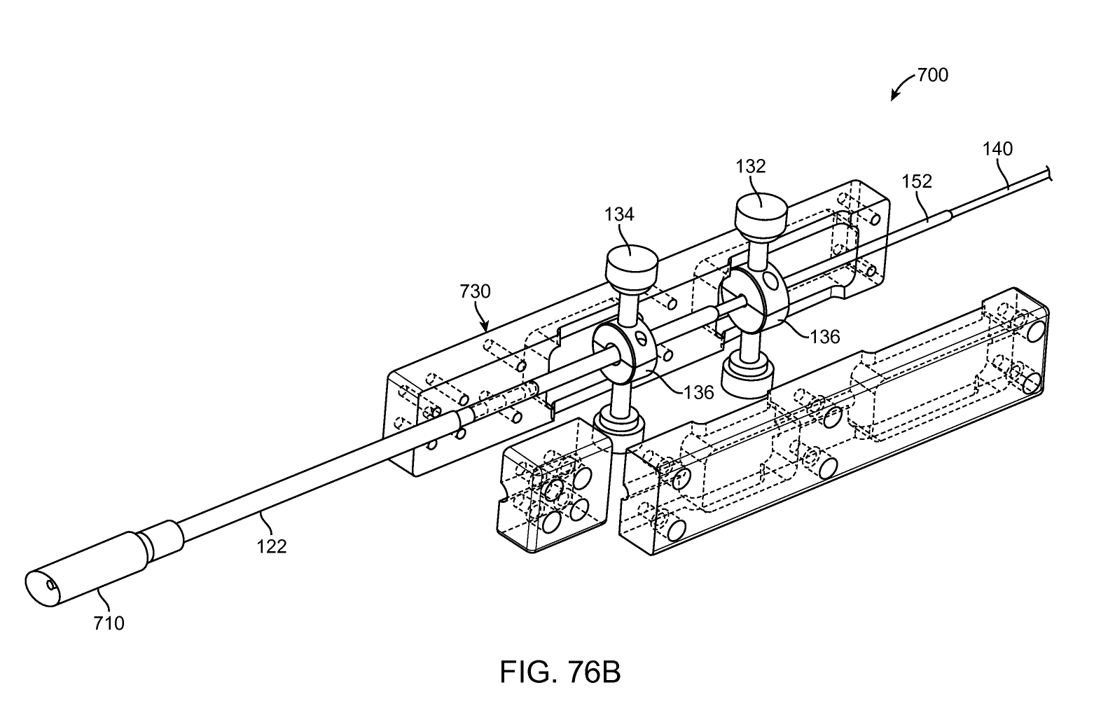

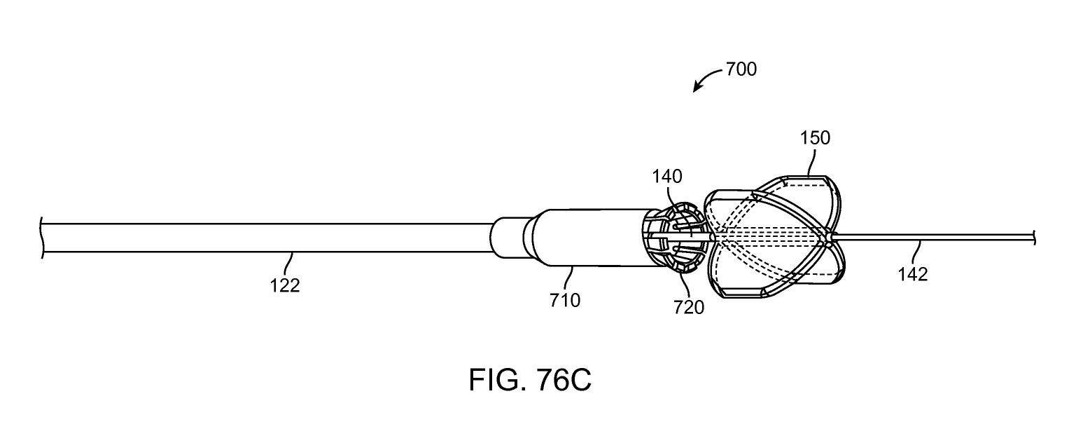

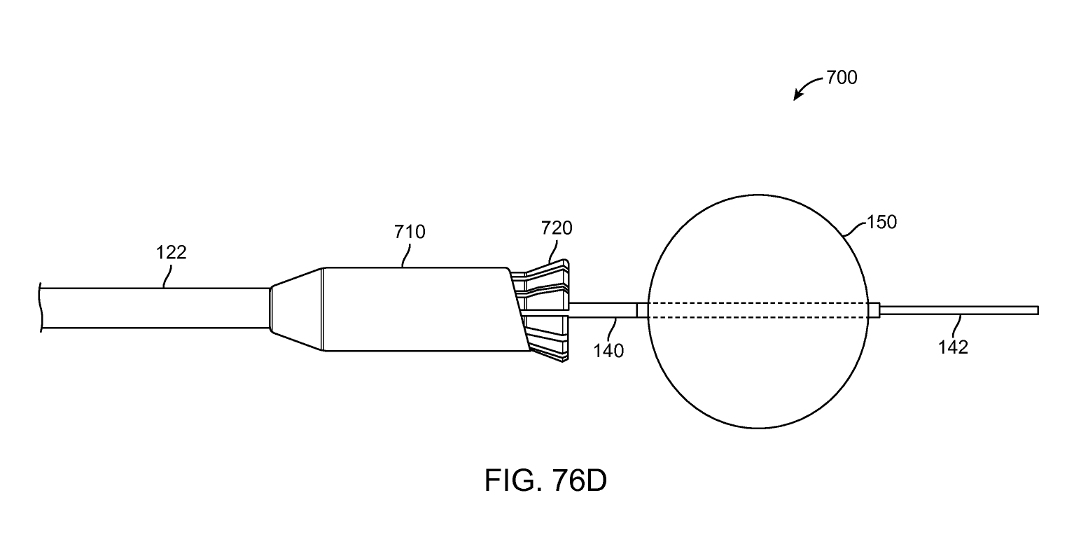

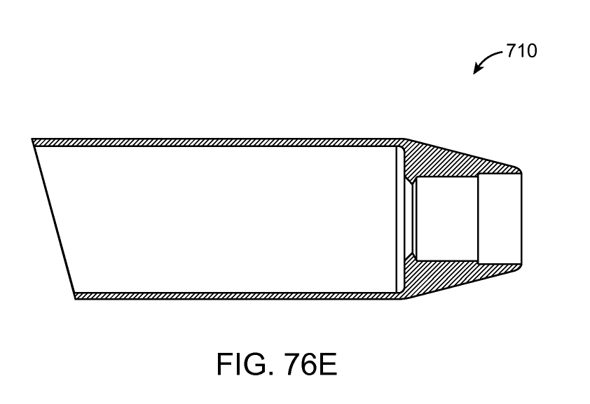

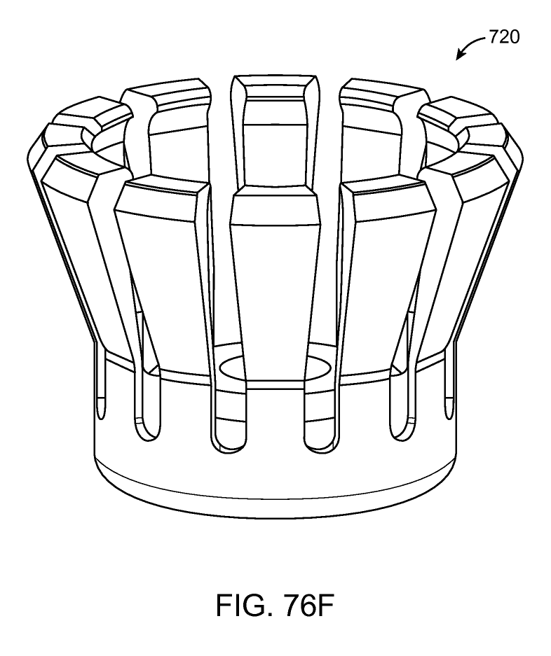

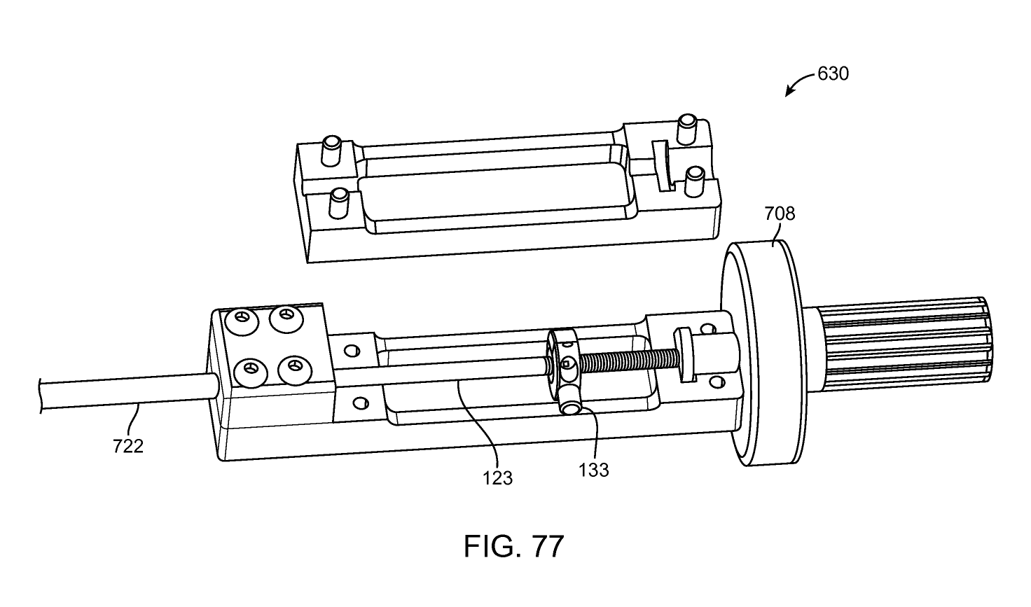

A handle 730, as shown in FIGS. 76A-76B, may be located on or attached to the proximal end 106 of the delivery device 700. However, although the handle 730 is shown on the proximal end 106, it is anticipated that the handle 730 may be located anywhere along the length of the delivery device 700 or may remotely or wirelessly control the delivery device 700. The handle 730 may be ergonomically designed and may fit easily into either the right or left hand of the user. It is anticipated that handle 130 (as shown in FIGS. 72-75), 630 (as shown in FIG. 77), or 730 (as shown in FIGS. 76A-76B) may be used with the delivery system.

The handle 130, 630, and 730 may serve as the user's (e.g. the endoscopist's) primary control interface to control the positions and functions of the delivery device 100 or 700. The handle 130 may have a various controls to, for example, control the movement of the implant positioner 120 and, thus, the deployment of the implant 10 along the distal end 104 of the delivery device 100 or 700. The handle 130 may also be used to control the position and/or rotation of the capsule 110.

Various applied forces and torques applied at the handle 130 may be transmitted or transferred to more distal elements of the delivery device 100 through a flexible lumen or insertion tube 122 securely attached or anchored to the handle 130. For example, axial forces applied at the handle 130 may result in the insertion or retraction of insertable elements (such as the pusher coil 102 or the implant positioner 120). Torque applied at the handle 130 may result in rotation of insertable elements of the delivery device 100. A strain relief may be located at the interface between the handle 130 and the insertion tube 122 to prevent damage to the insertion tube 122 (e.g. kinking) and/or handle 130 when bending forces are being imparted to either element.

The various controls may have a pusher collar 136 to control the movement and locking of the various mechanisms. According to one embodiment, the pusher collar 136 may be clamped or compressed along the movable lumen or shaft to prevent the movement. The handle 130 may also have a locking mechanism to prevent movement within certain components.

The handle 130 may be produced or manufactured using rapid prototyping techniques (e.g. FDM) or other manufacturing techniques, such as machining (either manual or CNC) or using mass-produced parts (e.g. injection molding).

According to one embodiment, the handle 130 may have an implant positioner or piston knob, lock, or control 134 to control the advancement and release of the implant 10 at the distal end 104 of the delivery device 100. The implant positioner control 134 may control relative axial motion between implant 10 and the capsule 110. For example, the implant positioner control 134 may control the movement of the implant positioner 120 through the insertion tube 122.

According to one embodiment as shown in FIG. 76B, the implant positioner control 134 may be a linear slide, which the user (e.g. the endoscopist) may move forward or backward to advance or retract, and then subsequently release the implant 10. The implant positioner control 134 may also be configured to be selectable between a sliding or a rotating action. For example, the implant positioner control 134 may be configured to slide linearly (e.g. when gross axial motion is desired) or to rotate (e.g. when fine axial motion or a mechanical advantage is desired) to control the position and release of the implant 10. A balloon knob, lock, or control 132 may also include the various embodiments of the implant positioner control 134.

Alternatively or additionally, as shown in FIG. 77 the handle 630 may have the rotating device 708 to gradually adjust the axial position and/or release of the implant 10. A balloon control 133 may also be used within the handle 630. The rotating device 708 may advance the implant 10 by turning a screw thread, which may provide a mechanical advantage, fine control of the components, and reduce the required efforts, as well as improve the fine control of the implant's position and release. According to another embodiment, the rotating device 108 or 708 may rotate the pusher coil 102 (as shown in FIGS. 72-75 and FIG. 77), thereby elongating or reducing the length of the pusher coil 102 to control the position of the implant 10. It is anticipated that either rotating device 108 or 708 may be used. For any configuration, the controls 134 and 108 may be locked to fix the relative position of the implant 10 within the capsule 110.

According to one embodiment, relative axial position of an axially positionable balloon 150 may be controlled by the balloon control 132 on the handle 130. The axial and rotational position of the balloon 150 may be locked by means of a control element incorporated into the handle 130. According to one embodiment, when the balloon control 132 is loosened, a balloon shaft 152 (and, therefore the balloon 150) may be moved freely relative to other element of the delivery device 100. For example, the balloon shaft 152 may be axially moved in or out of the delivery device 100 in order to advance or retract the balloon 150 at the distal tip of the delivery device 100. The balloon shaft 152 may be rotated in order to cause the balloon 150 to similarly rotate. When the balloon control 132 is tightened, the relative position of the balloon 150 may be locked and the applied axial forces or torques may not result in changes to the balloon's relative position.

The implant positioner control 134 and the balloon control 132 may be constructed using a variety of different techniques (e.g. machining techniques, mass-production approaches, or injection molding) and with a variety of different materials, including but not limited to machined metallic components (e.g. machined stainless steel) or injection molded plastic components.

The insertion tube 122 may connect the handle 130 and the capsule 110 and may extend from outside the body of the patient to the desired deployment site within the patient. The insertion tube 122 may be used to house or contain certain components or wires, such as the balloon shaft and/or the actuation tube, thus allowing the distal end of the delivery device to be manipulated by the handle 130.

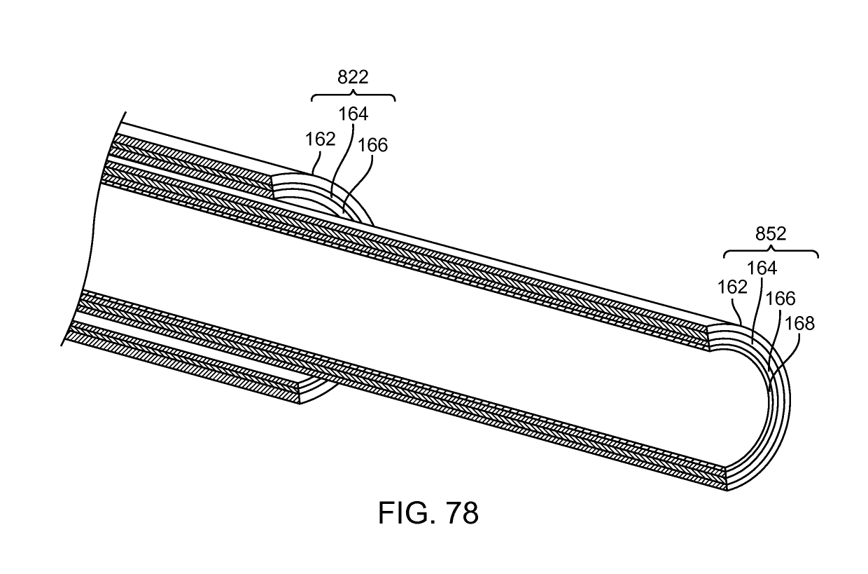

According to one embodiment, an insertion tube 822 may include of a number of coaxial, concentric elements, as shown in FIG. 78. For example, the insertion tube 822 may include a balloon shaft 852 that is concentric. The insertion tube 822 and the balloon shaft 852 may have an outer nylon jacket 162, a braid reinforcement 164, and a nylon inner liner 166 (from the outermost layer to the innermost layer). A PTFE liner 168 may further be located within the nylon inner liner 166. According to one embodiment, the outer and inner diameters of the insertion tube 822 may be approximately 1.9 and 1.5 mm, respectively. The outer and inner diameters of the balloon shaft 852 may be approximately 1.32 and 0.94 mm, respectively.

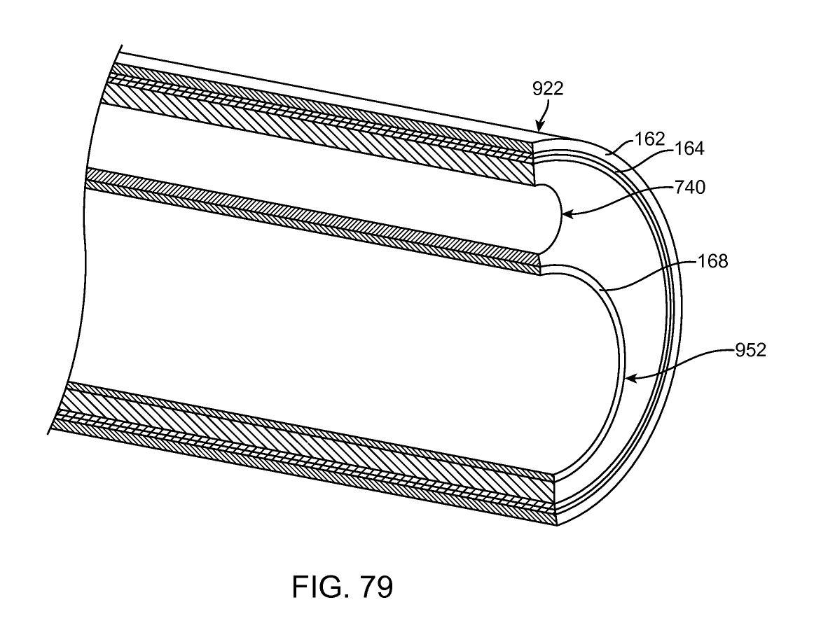

According to another embodiment as shown in FIG. 79, the component are not arranged coaxially and may be positioned next to each other as a dual lumen or a multi-lumen. For example, an insertion tube 922 may have a balloon shaft 952 next to a guidewire lumen 140. A dual lumen extrusion may maintain the relative positioning of the lumens. According to one embodiment, the outer and inner diameters of the insertion tube 922 may be approximately 1.9 and 0.9 mm, respectively. Although the balloon shaft 952 and a guidewire lumen 740 are shown in FIGS. 78 and 79, it is anticipated that other lumens, shafts, or tubes may be arranged coaxially or as a dual lumen, such as the actuation tube. It is anticipated that the guidewire lumen 140 or 740, the balloon shaft 152, 852, or 952, and that the insertion tube 122, 722, 822, or 922 may be used.

The length of the insertion tube 122 may vary depending on the desired use. According to one embodiment, the insertion tube 122 may be between 100-130 cm long. According to another embodiment, the insertion tube 122 may be approximately 125 cm long. The outer diameter and wall thickness of the insertion tube 122 may be approximately 5 mm and 0.5 mm wall, respectively. The insertion tube 122 may further be pushable, pullable, torquable, and kink resistant.

The outermost layer of the insertion tube 122 may be a flexible, thin-walled, sealed, biocompatible tube. The outermost layer may resist kinking, transmit torque well, resist excessive compression (e.g. when subjected to compressive loading), and may not elongate excessively (e.g. when subjected to tensile loading). Accordingly, the outermost layer of the insertion tube 122 may be constructed out of a single material, such as a coiled stainless steel wire, Pebax, rubber, silicone, Viton, fluoropolymers, and polypropylene. Alternately, the outermost layer may be constructed using more than one component, material, and manufacturing technique, including, but not limited to, metal or non-metallic braids, ribbons and/or coils, polymer layers, laminations, or co-extrusions. According to one embodiment, the outermost layer may include layers of metal ribbon coils, braids, and polymeric sheaths that are reflowed to join the components together.

The outer diameter of the insertion tube 122 may be minimized to facilitate side-by-side positioning with an endoscope in the body (in which case the outer diameter may be 5 mm or less). According to another embodiment, the outer diameter of the insertion tube 122 may be sized to fit within the working channel of an endoscope (in which case the outer diameter may be 3 mm or less). The inner lumen of the insertion tube 122 may be sized to accommodate a variety of internal components. The inner surface may have low frictional characteristics to accommodate the relative motion of internal components, which may slide axially and/or rotate. The internal components may be in any order or configuration within the insertion tube 122.

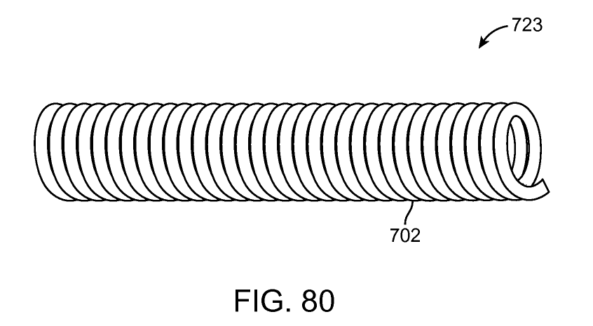

According to one embodiment, an actuation tube 123 or 723 may be positioned within the insertion tube 122 or 722 (as shown in FIG. 77). The actuation tube 123 or 723, as shown in FIGS. 77 and 80, may transmit commands, inputs, compressive or tensile loads, and forces (inputted by the user at the handle 130) to the components at the distal end 104 (e.g. the implant positioner 120) in order to control the position and deployment of the implant 10. The actuation tube may also be highly flexible, transmit torque well, resist kinking, resist excessive compression (e.g. when subjected to compressive loading), and may not elongate excessively (e.g. when subjected to tensile loading).

According to one embodiment as shown in FIG. 77, the actuation tube 123 may be a threaded guide. For example, one rotation of the actuation tube 123 (within the handle 130) may move the actuation tube 123 (and, thus, the implant positioner 120) forward 0.8 mm ( 1/32^{nd} of an inch) for fine control. According to another embodiment as shown in FIG. 80, the actuation tube 723 may be a coiled tube (e.g. pusher coil 702), which may be a wire bent into a coil around, for example, a mandrel. The pusher coil 102 or 702 may be “close wound” (e.g. there may be not space between successive turns of the wire). Pusher coil 102 or 702 may be used according to the desired configuration.

It is further anticipated that either actuation tube 123 or 723 may be used. According to one embodiment, the length of the actuation tube 123 may remain constant regardless of the tortuosity of its path. Alternatively, the construction and characteristics (e.g. length change vs. tortuosity, length variations) of the actuation tube 123 may match that of the insertion tube 122. If the lengths of the insertion tube 122 and the actuation tube 123 are constant regardless of tortuosity or each change by an equal amount, the position of the implant 10 in the capsule 110 may not be not unintentionally affected as the path of the insertion tube 122 changes. Accordingly, the actuation tube may be constructed using similar materials, processes, and techniques as that of the insertion tube 122, as described further herein. According to one embodiment, the handle 130 may have a diameter of approximately 0.635 mm.

According to one embodiment, the handle 130 may have a floating mechanism and a locking mechanism to help compensate for the changes in the length of the actuation tube 123 as the actuation tube 123 is bent through the bodily lumens to the delivery site. The floating mechanism may allow the actuation tube 123 to be linearly moveable (as the actuation tube 123 is being advanced into the patient) to compensate for length changes due to the coiled configuration and the tortuosity of the delivery path. Once the actuation tube 123 is positioned at the deployment site, the locking mechanism may be activated to stabilize the actuation tube 123. However, the locking mechanism may still allow the position of the actuation tube 123 to be controllably adjusted by the handle 130.

Depending on the configuration of the delivery device 100, the outer surface of the actuation tube 123 may move relative to the inner surface of the insertion tube 122. Additionally, the inner surface of the actuation tube 123 may house additional components, such as the balloon shaft 152. Therefore, the surface of the actuation tube 123 may use certain materials, coatings, and/or lubricants to minimize the friction.

According to one embodiment, the insertion tube 122 and/or the actuation tube may contain the balloon shaft 152 that is flexible and multi-lumen. The balloon shaft 152 may slide axially and/or rotate during use and may extend through the insertion tube 122. According to one embodiment, the balloon 150 and the balloon shaft 152 may be inserted into the delivery device 100 separately from the other components within the delivery device 100. The balloon shaft 152 may be a small, hollow tube to control the position of the balloon 150 at the distal end 104. The distal end of the balloon shaft 152 may be atraumatic in order to prevent unintentional injury to the body of the patient.

The balloon shaft 152 may incorporate one or more inner lumens, such as the guidewire lumen 140. The multiple lumens may be either arranged concentrically or as separate lumens. A separate lumen may be incorporated to introduce, transmit, withdraw, or remove a working fluid (e.g. air or saline) from a fluid or inflation port 154 (and seal) at the proximal end 106 of the balloon shaft 152 to the balloon 150 at the distal end 104. The fluid pressures may also be controlled at the inflation port 154 (as shown in FIG. 76A) to control the inflation or deflation of the balloon 150. The degree of inflation of the balloon 150 may be controlled by the flow of the working fluid and its pressure at the inflation port controls.

According to one embodiment, the balloon shaft 152 may have a diameter of approximately 1.27 mm. The balloon shaft 152 may further accommodate a guidewire 142 (or guidewire 742, 744, or 746). Therefore, the diameter of the inner lumen of the balloon shaft 152 may range between 0.9 to 1.14 mm. The proximal end of the balloon shaft 152 may extend past the proximal end of the handle 130.

The balloon shaft 152 may be constructed of a single material (e.g. Pebax, Rilsan, or Nylon) or with a number of components, materials, and manufacturing techniques (e.g. metal or non-metallic braids, ribbons, and/or coils; polymer layers; laminations; or co-extrusions). According to one embodiment, the outer surface of the balloon shaft 152 may move relative to the inner surface of the actuation tube and the inner surface of the balloon shaft may house additional components. Therefore, the balloon shaft 152 may be constructed out a variety of materials, coatings, or lubricants that minimize the friction.

Further, the balloon shaft 152 may incorporate a braid or coil layer for reinforcement and strengthening. The additional layer may improve tensile strength and kink resistance without excessively sacrificing flexibility. In such a braid-reinforced construction, a braid including of stainless steel wire (for example, 30 PPI, 0.001″×0.005″, 16 carriers, half load) may be used. While this construction is representative of braids that may be appropriate for the application, many other braid constructions are anticipated and may be used.

According to one embodiment, the balloon shaft 152 may have at least one extrusion and/or thin-wall liner. The extrusions may be reinforced with a braid or coil for flexibility, tensile strength, and kink resistance. In order to construct the balloon shaft 152, a braid may be advanced over an extrusion and another extrusion may be laminated on top to encapsulate the braid between the layers. PTFE-coated mandrels may be used during lamination for support and to keep the inner diameter of the balloon shaft 152 open. The PTFE coating may also help remove the balloon shaft 152 from the mandrels after processing. PTFE liners, such as guidewire lumens, may be incorporated into the inner diameter of the balloon shaft to make the surface more lubricious. According to one embodiment, the balloon shaft 152 may incorporates a dual-lumen extrusion with approximately a 1.04 mm major inner diameter, a 0.4 mm minor inner diameter, and a 0.076 mm wall thickness. The balloon shaft 152 may further have a polyimide liner, a stainless steel braid (30 PPI braid, 0.001″×0.005″, 16 carriers, half load), and an outer jacket extrusion (Nylon 12, 0.074″ ID, 0.002″ wall).

According to one embodiment, the relative motion between the implant 10 and the balloon shaft 152 may be facilitated through use of the tube 92. The tube 92 may keep the central axis of lumen of the compressed implant 10 open or free and may further reduce or minimize the impact of friction between the balloon shaft 152 and the implant 10 (in the tightly packed, compressed configuration 210) that surrounds the balloon shaft 152. The tube 92 may act as a spacer that surrounds the balloon shaft 152, allowing the balloon shaft 152 to slide axially with minimal friction within the compressed implant 10. According to one embodiment, the deflated balloon 150 may be delivered through the tube 92 before, after, or during deployment. The deflated balloon 150, extended through the tube 92, may prevent the tube 92 from falling out into the body after deployment.

The tube 92 may be a short, tubular element with an inner diameter slightly larger than the outer diameter of the balloon shaft 152 and may exhibit low friction relative to the balloon shaft 152, such that the balloon shaft 152 may slide freely within the tube 92. According to one embodiment, the inner diameter of the tube 92 may be approximately 1.9 mm. The tube 92 may have minimal wall thickness (to minimize the implant diameter) and adequate stiffness and structural integrity to remain patent and to prevent the compressive radial forces of the implant 10 from collapsing the balloon shaft 152. The tube 92 may be made out of variety of materials, including but not limited to PEEK, Ultem, PTFE-coated steel, and stainless steel.

According to one embodiment, the balloon shaft 152 may contain or house the guidewire 142, which may slide axially and/or rotate during use. The guidewire 142 may optionally be covered by the guidewire lumen 140, which may be internal to, and optionally integral with, the balloon shaft 152. However, the guidewire lumen 140 may be incorporated into or coaxial with a variety of elements and locations within the delivery device 100. It is anticipated that any of the guidewires 142, 742, 744, or 746 may be used.

A liner may be used inside the guidewire lumen 140 to reduce friction between the inner lumen surface and the guidewire 142 and to improve the integrity. For example, a polyimide liner may be used, or, alternately, the guidewire lumen 140 may be etched PTFE or FEP with approximately 0.001″ wall thickness.

The guidewire lumen 140 may extend from the handle 130 to the desired deployment site in the body. The length of the guidewire lumen 140 may depend on the desired type of procedure. The outer diameter of the guidewire lumen 140 may be small enough to fit beside or within the working channel of an endoscope within the body lumen(s). The inner diameter of the guidewire lumen 140 may be large enough to accommodate guidance or advancing elements, such as the guidewire 142 or the pusher coil 102.

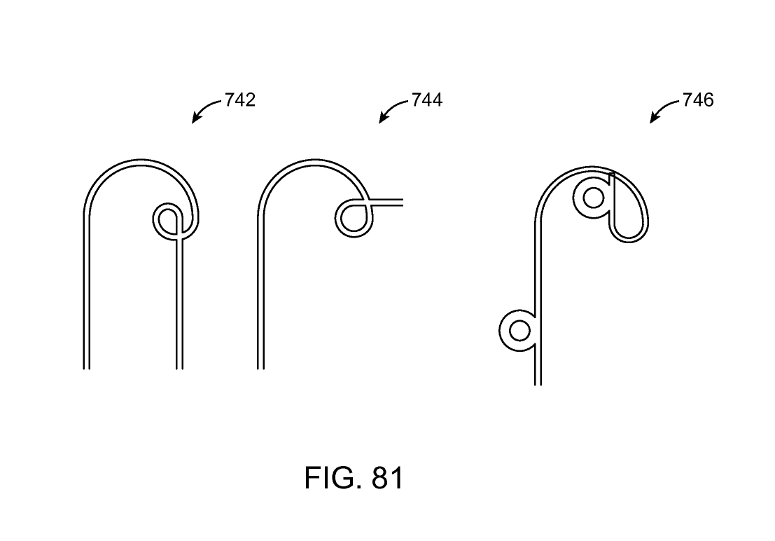

The guidewires 142, 742, 744, or 746, as shown in FIGS. 76A and 81, may be housed or contained within the guidewire lumen 140 and may be used to define a path to the deployment site. The guidewire 142 may facilitate the introduction, navigation, and control of the delivery device 100 during the delivery and deployment procedure. For example, the guidewire 142 may define the path that the delivery device 100 may follow when inserted into the patient. Therefore, the guidewire 142 may be placed into the body prior to the use of the delivery device 100 and may extend from the entry point of the endoscope or delivery device 100 (e.g. the mouth) to the desired implantation site (e.g. the gallbladder).

The guidewire 142 may be placed through the tool channel of an endoscope and the rest of the delivery device 100 may be subsequently inserted over the guidewire 142. In order to load the guidewire 142 into the delivery device, the proximal end of the guidewire 142 may be inserted into the distal end of the guidewire lumen (e.g. the terminus) and may be fed through the full length of the guidewire lumen until the guidewire 142 extends out of the proximal terminus of the guidewire lumen (in or near the handle 130).

According to one embodiment, the guidewire 142, 742, 744, or 746 may preferentially or automatically coil, bend, spring, or loop into a three-dimensional configuration or shape toward the distal end of the guidewire, as shown in FIG. 81, which may be inserted into or expanded within the distal cavity (e.g. the gallbladder). Once the guidewire 142 is released from the guidewire lumen 140 into the distal cavity, the guidewire 142 may automatically recoil, assuming its “low-energy” configuration. Accordingly, the coil on the guidewire 142 may expand, tense, or stretch the distal cavity to allow the implant 10 to be inserted or deployed easier and more accurately and consistently. The coil(s) may further prevent the guidewire 142 from pulling out of the distal cavity inadvertently.

According to one embodiment, the coils in the guidewire 142 may be approximately 30-46 cm from the distal end of the guidewire. The coils may forma a variety of different shapes. For example, the coils may create a circle with two turns of guidewire 142 and with a diameter of approximately 7.6 cm. The guidewire 142 may be plastically deformed or heat set in order to create the coil(s). According to another embodiment, the guidewire 142 may form a figure-eight configuration, a three-dimensional ball (with, for example, four lobes), a semi-circle shape, or an arrowhead shape (e.g. a smaller diameter toward the distal end and a larger diameter toward the proximal end).

The guidewire 142 may be, for example, a flexible or elastic, single-core, nitinol wire. The guidewire 142 may be sourced separately and manufactured by a third party company or may be included as a component of the delivery device 100. According to one embodiment, the outer diameter of the guidewire 142 may range from 0.635 to 1 mm. According to another embodiment, the outer diameter may be approximately 0.9 mm. Therefore, the inner diameter of the guidewire lumen 140 may be at least 0.9 mm. The length of the guidewire 142 may be approximately 4.5 meters.

In order to minimize frictional forces, a low-friction coating or lubricant may be applied to the guidewire lumen 140 and/or the guidewire 142) to accommodate relative motion with other components, such as the inner lumen of the balloon shaft 152 or the guidewire lumen 140. According to one embodiment, the guidewire 142 may have a fluoropolymer and/or hydrophilic coating and the guidewire lumen 140 (or the balloon shaft 152) may be flushed with water prior to use. According to another embodiment, the guidewire lumen 140 or the balloon shaft 152 (or inner lumen surface) and/or the guidewire 142 (or outer guidewire surface) may be a low-friction material, such as PTFE or polymide. According to another embodiment, a low-friction coating, such as Parylene or PTFE, may be applied to the inner guidewire lumen surface and/or the outer guidewire surface.

As shown in FIGS. 76A and 76C-76D, the distal end of the insertion tube 122 may be attached to a proximal end of a capsule 710 through a variety of mechanical means, including but not limited to clamping collars, setscrews, pins, and/or adhesives (e.g. epoxies, UV cure adhesives, or cyanoacrylate).

As shown in FIG. 72, the capsule 110 may contain, retain, and transport the implant 10 in the compressed configuration 210 to the desired deployment site and may facilitate proper positioning and deployment. The capsule 110 may also contain the positive retention features, such as the implant positioner 120, and an advancing mechanism, such as the pusher coil 102. Further, the capsule 110 may include the deflated balloon 150. The implant 10 may be progressively advanced and released from the capsule by manipulating the controls located in the handle 130. The capsule 110 may attach with the insertion tube 122 through a variety of different mechanisms, including but not limited to clamping or adhesives.

The capsule 110 may be have a variety of different shapes, configurations, and features according to the desired use and as described further herein. The features may help the capsule 110 traverse the opening in the tissue. Dilated openings in tissue may relax after the dilation instrument (e.g. the balloon 150) is removed. Further, the fit between the inner diameter of the opening and the outer diameter of the capsule 110 may be tight since additional dilation may decrease the effectiveness, function, and retention of the implant 10. Further, the capsule 110 may approach the dilated opening in the tissue walls at an oblique angle, rather than normal to the tissue walls. Therefore, the capsule 110 may have features to help move or advance through the tissue opening. According to one embodiment, the outer and inner diameters of the capsule 110 may be approximately 10 and 8.9 mm, respectively.

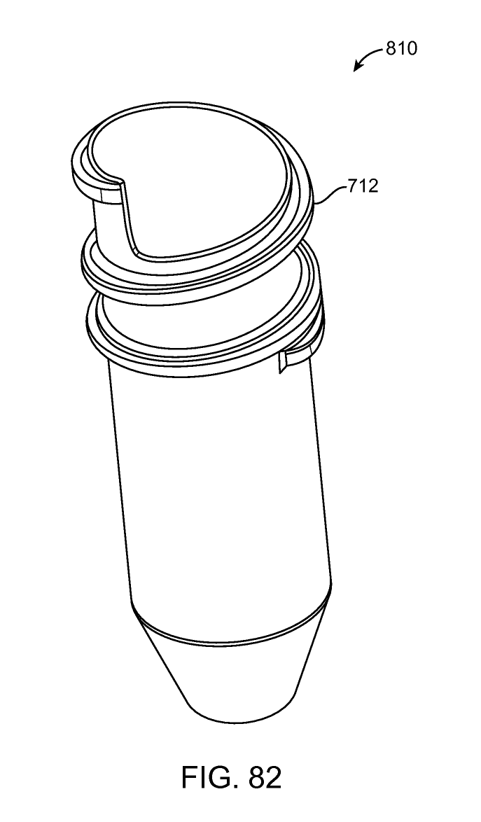

For example, according to one embodiment, the capsule 110 may have threads 112 or 712 along a portion of the distal end of the capsule 110 or capsule 810, as shown in FIGS. 72-75 and 82, that may wrap around and/or attach with a portion of the tissue. The threads 112 or 712 may be particularly beneficial to provide resistance and to atraumatically grab onto and align multiple soft tissues. Further, due to the anatomy of the body and the tension of the guidewire loops, the tissue openings may be misaligned and stretched into a slit, rather than a hole. Accordingly, the threads 112 or 712 may grab to the edge of the tissue openings. Threads 112 or 712 may be used according to the desired configuration.

By rotating or screwing the capsule 110 proximal to the tissue or within the tissue openings, the edges of the tissue openings may thread around or over the threaded leading edge of the capsule 110, aligning the tissue openings and sandwiching the tissue between the threads 112 and atraumatically securing the tissue walls over the distal end of the capsule 110 (without snagging the tissue). The threads 112 may both bring the two cavities or walls together and may align the openings within the tissue walls.

The number, pitch, and profile of threads 112 may vary according to the desired configuration. According to one embodiment, the threads 112 may wrap around the capsule 110 approximately 1.5 times. The threads 112 may have a variable or a regular pitch. For example, the threads 112 may have a relatively coarse pitch toward the distal end of the capsule 110 (to initially attach with the tissue easier) and a relatively fine pitch toward the proximal end of the capsule 110 (to more securely hold the tissue). The threads 112 may optionally pinch together at the end to prevent the tissue from sliding over the remainder of the capsule 110.

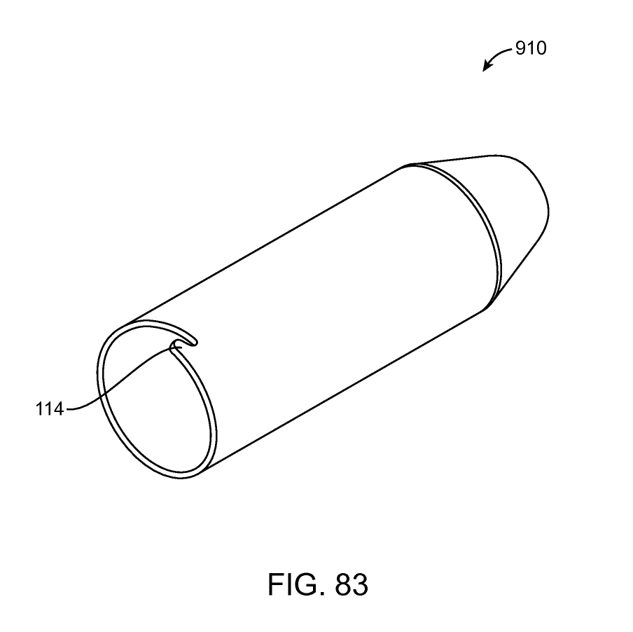

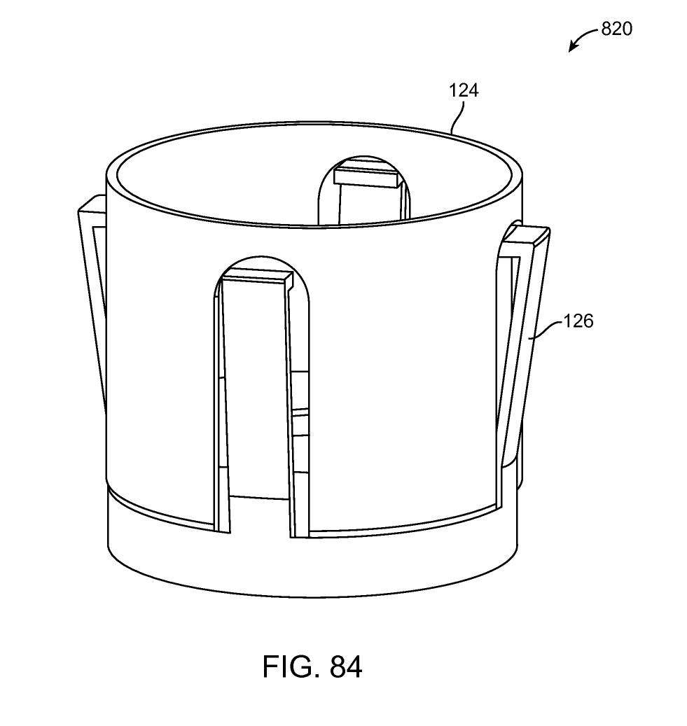

Alternative or additional to the threads 112, as shown in FIG. 83, the leading edge of the capsule 910 may have a lip, step, or hook 114 to hook or snag on an edge of the tissue wall as the capsule 910 is rotating toward the tissue, thus preventing the tissue from migrating or moving away from the capsule 910 and allowing the capsule 910 to easily move through the tissue openings.

According to another embodiment, as shown in FIG. 76E, the capsule 710 may be generally cylindrical along the length. The distal rim of the capsule 710 may be beveled (as shown in FIG. 76E) or configured at an angle other than 90° (e.g. not perpendicular to the longitudinal length of the capsule 110) to the capsule walls to help gradually move the capsule 710 through the tissue opening. According to one embodiment, the angle may be relatively small (e.g. approximately 15°), resulting in a slightly extended and pointed on the distal tip of the capsule 710. The distal tip may, however, not be sharp. The slight extension may facilitate the capsule 710 to be initially inserted through the dilated opening. For example, the extension may move through the opening first and at least a small portion of the capsule 710 may have to be extended further into the distal lumen.

The capsule 110 may be relatively thin-walled and may be constructed out of a variety of materials, including plastic or metal. The capsule 110 may be manufactured through a variety of techniques, such as injection molding for high-volume production.

The capsule 110 may optionally include a pusher piston, which may help eject and deploy the implant 10 out of the capsule 110. The pusher piston may attach with a portion of the insertion tube 122 (such as the actuation tube 123). According to one embodiment, the pusher piston may be a concave, flat pusher. According to another embodiment, the pusher piston may be a cup to radially constrain a portion (e.g. the flange 12) of the implant 10 and prevent the flange 12 from prematurely deploying. The pusher piston may further reduce the friction within the capsule 110.

Alternatively or additionally, the capsule 110 may include the implant positioner 120 to control the position, expulsion, and deployment of the implant 10. For example, the implant positioner 120 may hold the implant 10 in place within the capsule 110 and may allow the one of the flanges to deploy at a time by moving the implant 10 within the capsule 110. The implant positioner 120 may be configured to hold or grasp one or both of the flanges 12 or 14 at least partially within the capsule 110. The implant positioner 120 may prevent at least a portion of the implant 10 from sliding against the inner surface of the capsule 110 and may securely hold the implant 10 while the implant 10 is advanced during deployment.

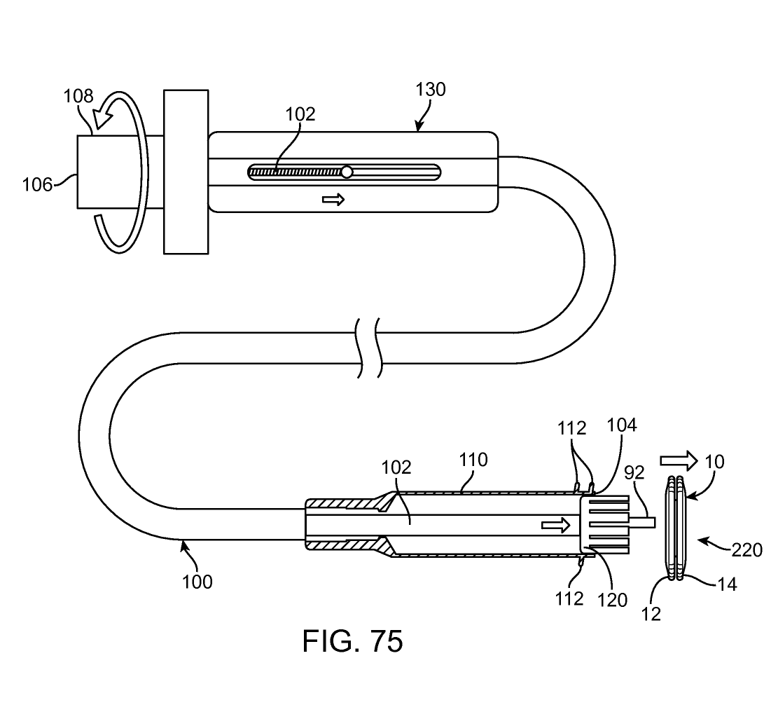

The implant positioner 120 may be movable within the capsule 110 between at least a first position and a second position. When the implant 10 is loaded into the capsule 110 and is ready to be delivered and deployed, the implant positioner 120 is retracted back toward the proximal end of the capsule 110, thereby securing the flange 12 within the implant positioner 120 and creating space to accommodate the flange 14 within the capsule 110 (e.g. the first position, as shown in FIG. 72). Once the delivery device 100 has been inserted into the patient and the capsule 110 is in the desired location to deliver the implant 10, the implant positioner 120 may be pushed forward by manipulating the implant positioner control 134 at the handle 130 into the second position (as shown in FIG. 75), where the implant positioner 120 at least partially extends beyond the distal end of the capsule 110 and releases the flange 12. The actuation tube may transmit the forces from the implant positioner control 134.

The implant positioner 120 may be shaped according to the desired configuration. According to one embodiment, the implant positioner 120 may be a simple flat disc to push the implant 10 out of the capsule 110. According to another embodiment, the implant positioner 120 may be a cup with side walls that may surround and contain the implant 10 while inside the capsule 110.

According to one embodiment, the implant positioner 120 may have at least one small, radially-arranged, elastically flexible, grasping finger or pincer around the perimeter of the implant positioner. The fingers may squeeze inward when the implant positioner 120 is inserted into (and constrained by) the capsule 110 in the first position. More specifically, the inner surface of the capsule wall may force the grasping fingers inward to positively grip, secure, or retain one of the flanges (e.g. flange 12) while the implant 10 is loaded in the capsule 110. The grasping fingers may also control the deployment of the implant 10, such that the flanges 12 and 14 may be deployed separately. When the implant positioner 120 is advanced out of the capsule 110 from the first position to the second position, at least a portion of the grasping fingers may expand radially outward, which causes them to relax their grip on the implant 10, allowing the other flange (e.g. flange 12) to expand and deploy.

According to another embodiment as shown in FIG. 76F, an implant positioner 720 may include a radial array of grasping fingers, each angled radially outward in their relaxed (unconstrained) state. When the implant positioner 720 is inserted into the capsule 110, the inner surface of the capsule 110 may push the grasping fingers radially inward like a collet. The amount of grasping, or inward radial force, may be determined by the amount of inward radial displacement that occurs when the grasping fingers are inserted into the capsule 110 and the shape of the fingers. The fingers may be tapered and may have a hook or lip on the end to further grasp the implant 10. The implant positioner 720 may include any number, size, spacing, and configuration of fingers. For example, the implant positioner 720 may have 8-16 fingers around the perimeter.

According to another embodiment as shown in FIG. 84, an implant positioner 820 may include two elements: a modified cup 124 with a series of slots in the side walls and a finger element 126 nesting with the cup 124. The fingers of the finger element 126 may extend through the slots of the cup 124 from the outside of the cup 124, decreasing the inner diameter of the cup 124. For example, when the implant positioner 820 is inserted into the capsule 110, the fingers may be pushed radially inward when the fingers contact the inner surface of the capsule 110, which may grasp a portion of the implant 10. The fingers of the finger element 126 may align with the slots in the cup 124. However, there may be any number and size of fingers and slots according to the desired configuration.

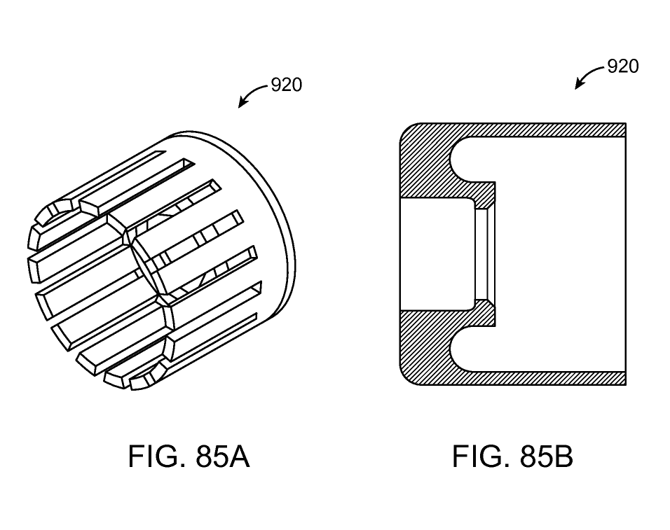

According to yet another embodiment as shown in FIGS. 85A-85B, the elastically flexible fingers of an implant positioner 920 may be about parallel to the central axis (e.g. the z-axis) of the implant positioner 920 in the expanded configuration and may have a constant thickness. When the implant positioner 920 is extended from the capsule 110, the fingers may radially expand slightly (e.g. approximately 3.8 mm) for easier insertion of the implant 10 into the implant positioner 920. The spring force of the fingers may be less than the spring force of the implant positioner 920. For example, as the implant 10 is deployed, the spring force of the implant 10 may push the fingers at least partially outward, thereby releasing the implant 10. Therefore, the fingers may be at least partially compliant or flexible.

It is anticipated that any of the configurations and components of the implant positioners 120, 720, 820, and 920 may be used with the implant positioner according to the desired configuration and use. The implant positioner may be constructed out of a variety of materials that may allow the implant positioner to grasp the implant 10 and may remain in the elastic deformation mode. For example, the implant positioner may be made out materials including, but not limited to, stainless steel, peek, Nitinol, ABS, Delrin, polycarbonate, PTFE-filled acetyl, or a material suitable for injection molding.

According to one embodiment as shown in FIGS. 76C-76D, the delivery device 700 may have an expandable mechanism, such as the balloon 150, on the tip of the distal end 104. The balloon 150 may be deflated (as shown in FIG. 76C) or inflated (as shown in FIG. 76D), regardless of the position of the implant positioner or the implant.

The position, deployment, and amount of expansion (e.g. inflation) of the balloon 150 may be manipulated by certain controls on the handle 130. The balloon 150 is axially repositionable relative to the other elements of the delivery device 100 by advancing or retracting the balloon shaft 152 at the proximal end of the handle. The balloon 150 may also be rotated. The position of the balloon 150 may be clamped or locked by features incorporated into the handle 130. The balloon 150 may be inflated (with, for example, gas or liquid (e.g. saline)) and deflated through the inflation port 154 on the insertion tube 122. According to one embodiment, the saline may include a contrasting material to allow the balloon to be visualized under fluoroscopy.

The balloon 150 may act as an anchor and ensure retention and control within the distal lumen (e.g. the gallbladder) throughout the procedure, preventing the delivery device 100 from being unintentionally withdrawn while placing the implant 10. The balloon 150 may provide shape and structure within the distal lumen and may prevent the flange 14 from inadvertently being pulled through the tissue opening during deployment by providing resistance or tension against the tissue. The balloon 150 may further be used to facilitate the introduction of the capsule 110 through the opening and across the tissue walls by pushing the tissue walls over the outside of the capsule 110. The balloon 150 may additionally be used to manipulate sections of the implant 10 to adjust the positioning of the implant 10.

According to one embodiment, the balloon 150 may be a compliant, spherical or round balloon with a relatively low durometer. According to another embodiment, the balloon 150 may be relatively flatter along two sides. The balloon 150 may be located at, or as near as possible to, the distal end of the balloon shaft 152. The diameter of the balloon 150 may range between 30 to 50 mm. According to one embodiment, the balloon 150 may be a urethane balloon, relatively inelastic (for additional structure and strength), and may be thermally or adhesively bonded to the balloon shaft 152.

The deflated balloon 150 may be folded or collapsed into a uniform configuration in order to minimize the outer diameter or profile and to prevent any edges of the balloon 150 from catching on the tissue during delivery. For example, the balloon 150 may be pulled back on itself and folded into four pleats or quadrants. The balloon 150 may further be deflated with a vacuum.

According to one embodiment, a proximal thermal bond and an adhesive distal bond may be used to bond the preferred balloon configuration. For example, a hot box may be used to reflow a portion of the balloon 150 down to the outer diameter of the balloon shaft 152 and to thermally bonded to the distal end of the balloon shaft 152. The balloon 150 may be inverted by folding the balloon 150 back over itself towards the distal end, so that the proximal bond may be under the balloon 150 membrane. The distal balloon neck may also be reflowed down to the outer diameter of the balloon shaft 152 using a hot box, and then adhesively bonded to the balloon shaft 152 using UV-cured adhesive. The balloon bond length (e.g. the distance between the proximal and distal bonds) may be shorter than the natural length of the balloon 150, creating a disc shape when inflated.

After the implant 10 has been loaded into the delivery device 100 (as described further herein), the implant 10 may be delivered and deployed within the body of the patient. FIGS. 72-75 and 86A-86E depict the implant 10 being deployed from the delivery device 100 in accordance with one embodiment. The delivery device 100 may further maintain the compressed configuration 210 of the implant 10 until the implant is properly positioned.

Prior to delivering the implant 10, an endoscope (such as an EUS endoscope) may be introduced to the delivery site. Under ultrasound guidance, an FNA (fine needle aspiration) needle may extend through the tissue wall(s), from the first bodily lumen (e.g. the duodenum) to the second bodily lumen (e.g. the gallbladder), puncturing or making a cut, slit, hole, tract, access point, incision, or opening within at least one tissue to connect at least two cavities. If there are multiple tissues for the implant 10 to secure, multiple tissues may be cut. However, according to another embodiment, the delivery device 100 may utilize naturally-made apertures within the tissue. The gallbladder may optionally be drained prior to delivering the delivery device 100.

The guidewire 142 may be introduced through the core of the FNA needle (or over the needle lumen) into the distal cavity (e.g. the gallbladder). As the guidewire 142 is released into the distal cavity, the guidewire 142 may assume a coiled configuration (or be coiled) approximately two times within the distal lumen, which may secure or hold the guidewire 142 within the gallbladder. Alternatively, the guidewire 142 may be about straight (outside of the body) and may automatically coil to match the curvature of the gallbladder as the guidewire 142 is advanced. The FNA needle may be withdrawn while the guidewire 142 may be left in place.

The deflated and/or folded balloon 150 (such as a dilation balloon) may be advanced over the guidewire 142 and through the working channel of the endoscope. The balloon 150 may be positioned through the opening and across the tissue wall(s). The balloon 150 may be inflated to dilate, expand, or enlarge the tissue opening to a size that will accommodate or accept the capsule 110. According to one embodiment, the tissue opening may be dilated to a diameter of approximately 12 mm. The balloon 150 may subsequently be deflated and withdrawn, leaving the guidewire 142 in place. However, it is anticipated that the balloon 150 may be kept within the distal cavity to help with deployment and positioning. The endoscope may also be withdrawn.

The delivery device 100 may subsequently be advanced over the guidewire 142. The delivery device 100 may be inserted through a natural or artificial opening of the body and may progress through at least one body lumen to the deployment site. For example, according to one embodiment, the delivery device 100 may be inserted through the mouth of a patient and may be advanced through the esophagus and stomach and into the duodenum. An endoscope may also be advanced in tandem or separately from the delivery device 100 for direct visualization of the position and progress of the delivery device 100 as the delivery device 100 reaches the deployment site. According to one embodiment, the deflated balloon 150 (which may optionally be a second balloon) may be delivered with the capsule 110 (e.g. within the capsule 110 or in front of the capsule 110).

At least the distal rim or distal end 104 of the capsule 110 may be advanced, traversed, or delivered through the opening(s) into the distal cavity (e.g. the gallbladder) to order to position and deploy the flange 14. The capsule 110, 710, 810, or 910 may further incorporate features to facilitate insertion through the opening(s) as shown in FIGS. 76E, 82, and 83 and as described further herein. For example, the capsules 110 or 810 with threads 112 or 712 may be rotated through the opening to grasp the tissue walls. Prior to advancing and rotating the capsule 110 forward through the tissue opening, the capsule 110 may be, for example, rotated backward about 1.5 to 2 times within the proximal cavity to ensure that the capsule 110 grabs the correct portion of tissue. It is anticipated that any of the components and characteristics of the capsules 110, 710, 810, or 910 may be used with the capsule according to the desired configuration.

According to another embodiment, in order to further help move the capsule 110 across the tissue opening, at least one of the cavities may be expanded with an expanding mechanism, such as the balloon 150 (which may be the same as or separate from the dilation balloon). The expanding mechanism may further be used to approximate, appose, or pull together the tissue walls of the first and second cavities by sandwiching or compressing the tissue walls between the expanding mechanism and a portion of the delivery device 100. For example, the balloon 150 may be advanced into the distal cavity and inflated (thereby securing the balloon 150 in the distal cavity). The balloon 150 may then be retracted or pulled back toward the distal rim of the capsule 110 to contact the tissue wall and secure and appose the tissue walls (of both the distal and proximal cavities) together between the distal rim of the capsule 110 and the balloon 150 (while the capsule 110 is being advanced through the tissue opening). The balloon 150 may help the capsule 110 advance into the distal cavity by pushing the wall of the distal cavity over the outside of the capsule 110. The balloon 150 may also provide resistance for the capsule 110 to advance through the tissue. The balloon 150 may further provide anchoring, shape, and structure within the distal cavity.

According to another embodiment, the deflated balloon 150 may positioned and secured partially within and partially extending out of the capsule 110. The balloon 150 may be inflated, which may form or bulge the balloon 150 around a portion of the distal end of the capsule and help atraumatically push the walls of the tissue over the capsule 110 to help advance the capsule 110 into the distal cavity. Once the distal rim of the capsule 110 is through the tissue walls and in the distal cavity, the balloon 150 may be deflated and advanced forward.

The delivery device 100 may progressively deploy the flange 14 and then deploy the flange 12 out of the capsule 110, such that the flanges 12 and 14 are released, opened, expanded, and/or deployed separately in their respective bodily cavities. Once at least the distal rim of capsule 110 is within the gallbladder, the flange 14 may first be released or deployed into the distal cavity (e.g. the gallbladder or the second cavity the deployment device may advance at least partially into) while the delivery device may retain full control over the lumen 16 and the flange 12.

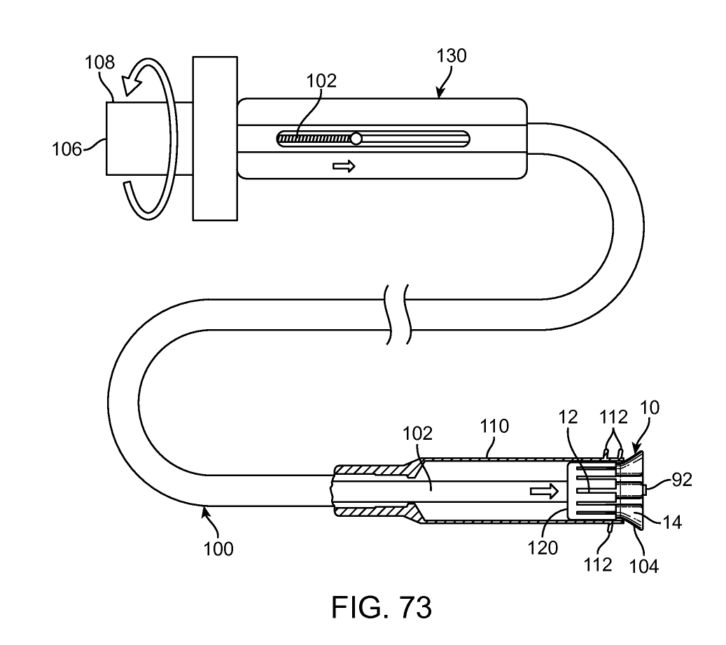

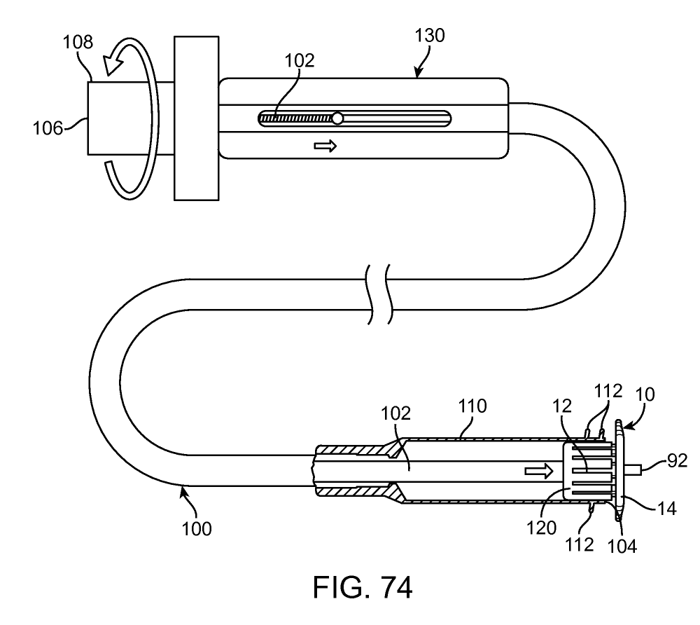

In order to release the flange 14, as shown in FIG. 72, the rotating device 108 may be rotated in order to elongate the pusher coil 102 or central lumen of the delivery device 100, which extends or advances the implant positioner 120 away from the proximal end 106 and towards the distal end 104 of the delivery device 100. Accordingly, the length of the pusher coil 102 or central lumen may control the position of the implant positioner 120 and the implant 10. The implant positioner 120 both moves and pushes the implant 10 in the same direction, releasing the flange 14 through the distal end 104 of the delivery device 100. Because the implant positioner only secures the flange 12 of the implant 10, the flange 14 is released and deploys separately from the flange 12, as shown in FIGS. 73 and 74. This allows the flange 14 to deploy within a separate body cavity from the flange 12. As the flange 14 is deployed, the flange 14 may spring outward and expand unconstrained into the deployed configuration 220 due to the spring force of the implant 10. As such, the width of the implant 10 increases and the length of the implant 10 decreases, thus at least partially exposing the tube 92. Meanwhile, the flange 12 may be contained within the implant positioner 120 within the capsule 110 in the compressed configuration 210.

Subsequently, the entire capsule 110 may be repositioned, withdrawn, unscrewed, or retracted through the tissue opening into the proximal cavity (e.g. the duodenum), such that the distal end 104 of the capsule 110 is next to the proximal-most lumen wall (e.g. the duodenum wall). The flange 12 may be released into the proximal cavity (e.g. the duodenum), thereby joining the two lumens (e.g. the gallbladder lumen and the duodenal lumen) and securing anchoring the tissue walls between the flanges 12 and 14.

In order to release the flange 12, the pusher coil 102 may be further extended and advanced toward the distal end 104 (due to the movement of the rotating device 108), thereby extending or pushing the implant positioner 120 beyond the distal rim of the capsule 110. Because the capsule 110 no longer constrains the implant positioner 120 and/or the implant 10, the flange 12 may expand and deploy due to the outward spring force of the implant positioner 120 and the implant 10. The spring force of the flange 12 may additionally help force the fingers of the implant positioner 120 open in order to be released and deploy. Thus, with both the flanges 12 and 14 deployed, the implant 10 assumes the deployed configuration 220. According to one embodiment, the implant positioner 120 may radially expand out of the end of the capsule due to the spring force of the implant positioner 120. Once both of the flanges 12 and 14 have been released, the entire implant 10 may be released or detached from the delivery device 100 and the implant 10 may be positioned across both tissue walls with both of the flanges 12 and 14 engaging the tissue walls of two body cavities. Accordingly, the balloon 150 may be deflated and the balloon 150, the delivery device 100, the endoscope, and the guidewire 142 may be removed or withdrawn from the patient. The implant positioner 120 may optionally be retracted back into the capsule 110 prior to withdrawing the delivery device 100 from the body.

However, it is anticipated that the implant 10 may be delivered with or through a variety of different devices, such as through the tool or working channel of an endoscope or an echo-endoscope. The implant 10 may also be delivered outside of an endoscope (the implant 10 may, for example, be delivered over a guidewire). An endoscope may optionally be positioned next to the guidewire to provide visualization and guidance while the implant 10 is being delivered and deployed. As described further herein, the shape and size of the implant 10 (in both the compressed configuration 210 and the deployed configuration 220) may be customized according to the size of the anticipated delivery device and the size of the body lumens through which the implant 10 must travel.

According to another embodiment, fluoroscopy may be used to visualize the delivery device 100 and deployment during any stage of the process to, for example, guide the delivery device 100, confirm the relative positioning of the components within the body, and/or confirm that the implant 10 has been properly deployed.

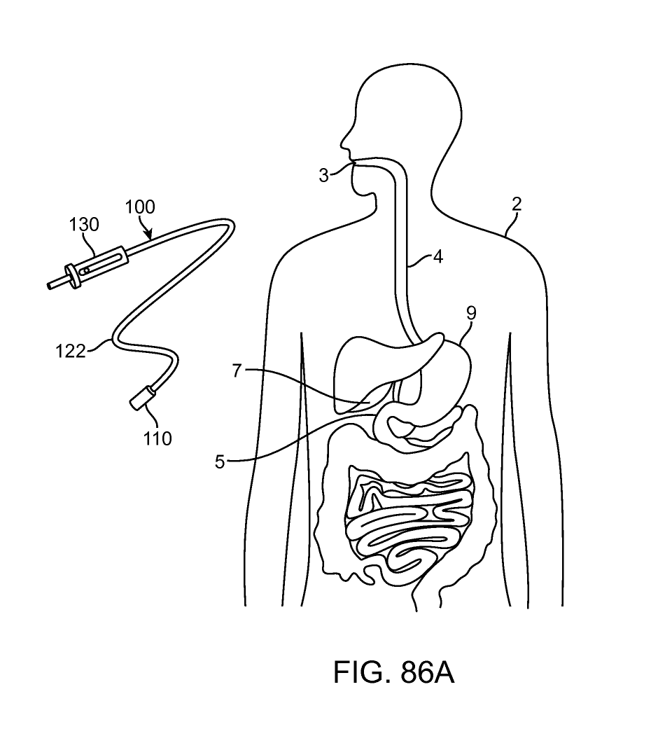

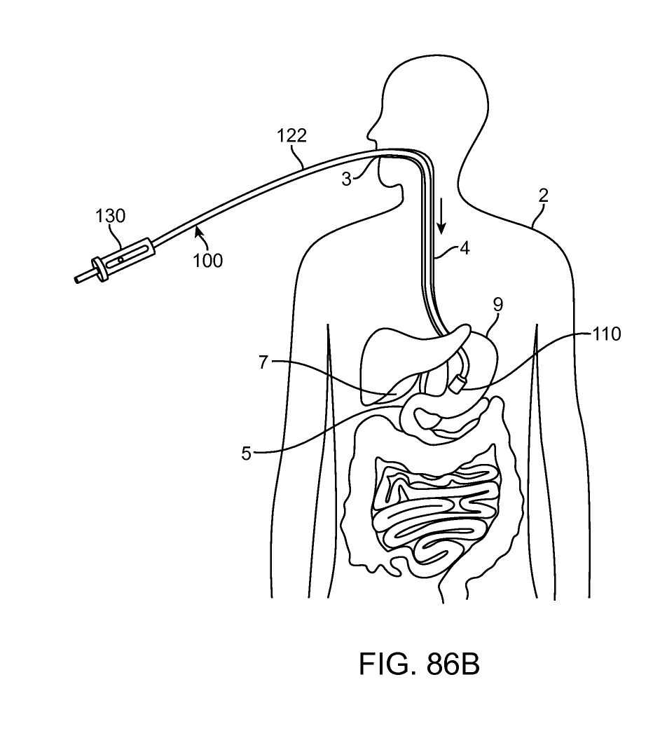

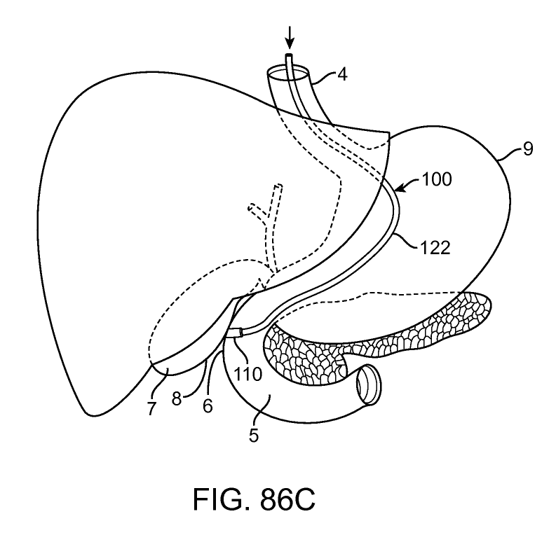

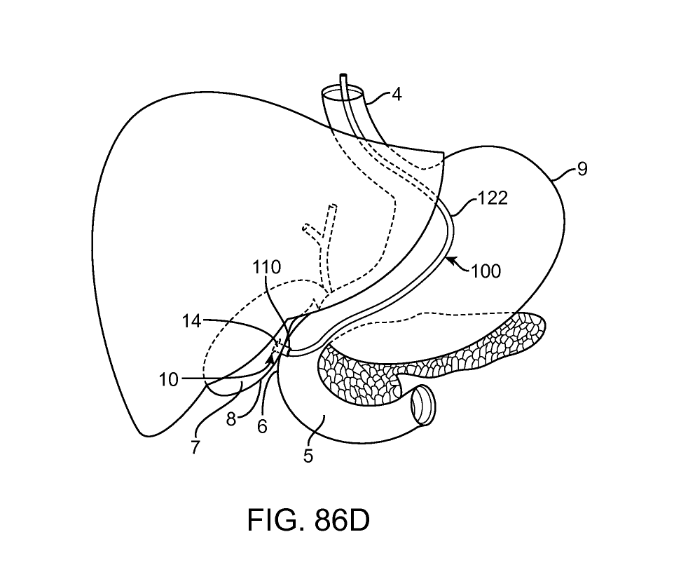

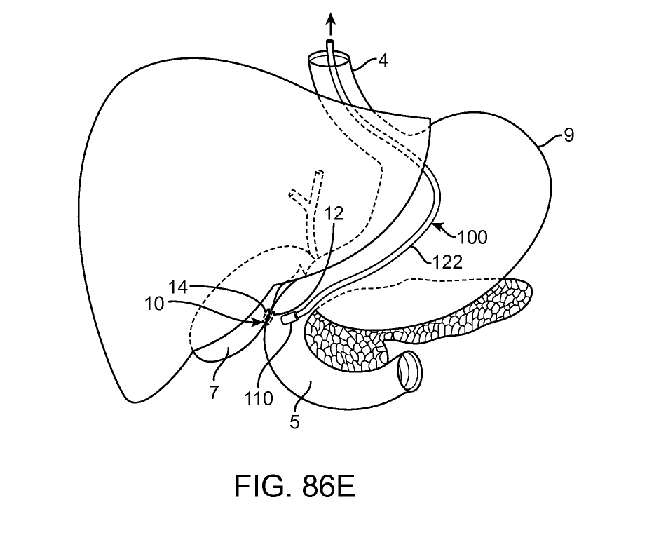

As shown in FIGS. 86A-86E, the implant 10 may be used to connect a duodenum 5 and a gallbladder 7. Once the implant 10 has been loaded into the delivery device 100 (as described further herein and as shown in FIG. 72), the delivery device 100 may be prepared to deliver and deploy the implant 10, as shown in FIG. 86A. The delivery device 100 may be advanced through a mouth 3 of a body 2 of a patient and into the esophagus 4 and the stomach 9, as shown in FIG. 86B. The delivery device 100 may be further progressed into the duodenum 5 and toward the gallbladder 7, as shown in FIG. 86C. While the implant 10 is being delivered (and before deployment), the delivery device 100 may hold the implant 10 in the compressed configuration 210, as shown in FIG. 72.

As described further herein, the capsule 110 may be advanced through the duodenum tissue 6 and through the gallbladder tissue 8, such that at least a portion of the capsule 110 is situated within the gallbladder 7. Once the capsule 110 is properly situated, the delivery device 100 may deploy the flange 14 of the implant 10 into the gallbladder, as shown in FIG. 86D. The flange 14 deployment is further shown in FIGS. 73 and 74. After deployment of flange 14, the delivery device 100 may retract the capsule 110 back into the duodenum 5 and may deploy the flange 12, thereby connecting the duodenum 5 and the gallbladder 7 as shown in FIG. 86E. The flange 12 deployment is further shown in FIG. 75.

Although the delivery device and various respective parts are referred to, it is anticipated that any of the components or embodiments of the delivery device may be combined and used to deliver and deploy any of the implants with any of the loading tools.

The embodiments disclosed herein allow an implant to be loaded and delivered into a delivery device for proper deployment and allow at least two body lumens to be connected with the implant. Besides those embodiments depicted in the figures and described in the above description, other embodiments are also contemplated.

As used herein, the meaning of “about,” “substantial,” “substantially,” “generally,” “approximately” is generally meant to be within +/−10% of the value it modifies, for example, within +/1.0% of the value it modifies. Also used herein, the meaning of “partially” is generally meant to be greater than about 25% to less than 100% of the term it modifies, for example, greater than 50% or 75% and less than 100%.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### Various Embodiments

According to one embodiment, a device for implantation in a body may include a body defining a lumen. The body may have a first end and a second end, and the lumen may define a central axis. The body may include a first flexible flange having a first base portion and a first angled portion. The first base portion may be generally or about perpendicular to the central axis and extend radially away from the first end. The body may further include a second flexible flange having a second base portion and a second angled portion. The second base portion may be about perpendicular to the central axis and extend radially away from the second end. The first and second angle portions may be generally angled toward each other.

In any of the embodiments, the first and second angled portions may have a narrower gap between them than the gap between the first and second base portions.

In any of the embodiments, the first and second angled portions may touch each other at least at one point between the first and second angled portions.

In any of the embodiments, the first and second flanges may be symmetrical mirror images of each other.

In any of the embodiments, the first and second flanges may be different shapes or sizes.

In any of the embodiments, the first flange and the second flange may be configured to fit at least one tissue in a patient between the first flange and the second flange.

In any of the embodiments, the lumen may be configured to connect at least two cavities within the patient and the first flange may be positioned within a first cavity and the second flange may be positioned within a second cavity.

In any of the embodiments, the at least one tissue may be held between the first and second flanges with a compressive force.

In any of the embodiments, the compressive force may be configured to reduce circulation within the at least one tissue.

In any of the embodiments, the compressive force may be configured to cause the at least one tissue to undergo necrosis.

In any of the embodiments, a length of the lumen may be at least a thickness of the at least one tissue.

In any of the embodiments, the device may be dissolvable within a patient after a period of time.

In any of the embodiments, the device may include a tracking mechanism.

In any of the embodiments, the second flange may be configured to be deployed within a distal cavity and the first flange may be configured to be deployed within a proximal cavity.

In any of the embodiments, the first flange and the second flange may be at least partially hollow.

In any of the embodiments, at least one of the first flange or the second flange may have a textured portion.

In any of the embodiments, the textured portion may be at least one groove.

In any of the embodiments, the textured portion may be at least one bump.

In any of the embodiments, the textured portion may be at a line perpendicular to the central axis.

In any of the embodiments, the textured portion may be faced away from at least one of the first flange or the second flange.

In any of the embodiments, the textured portion may be faced toward at least one of the first flange or the second flange.

In any of the embodiments, the body may be flexible.

In any of the embodiments, at least one of the body, first flange and second flange may be elastically deformable.

In any of the embodiments, the device may be elastically compressible.

In any of the embodiments, the first flange and the second flange may be folded away from each other and toward the central axis producing a folded configuration.

In any of the embodiments, the folded configuration may be adapted for delivery in at least one of a catheter or an endoscope.

In any of the embodiments, the lumen may include a one-way valve.

In any of the embodiments, the lumen may include a pressure relief valve.

In any of the embodiments, the lumen may include a plug, and the plug may prevent material from moving through the lumen.

In any of the embodiments, the device may be constructed out of a silicone material.

In any of the embodiments, the silicone material may be selected from a group consisting of at least one of silica, liquid silicone rubber elastomer, and dimethyl, methylhydrogen siloxane copolymer.

In any of the embodiments, the device may include a radiopaque material.

In any of the embodiments, the radiopaque material may be selected from a group consisting of at least one of barium sulfate, a nitinol ring, platinum radium markers, a braided stainless cable, and a mechanical feature.

In any of the embodiments, the concentration of the radiopaque material may be correlated to a weight of the device.

In any of the embodiments, the radiopaque material may be barium sulfate and the concentration of barium sulfate may be approximately 20% of the weight of the device.

According to another embodiment, a medical apparatus for forming a shunt between two tissues may include a tube having a first end and a second end, a first tissue engager may have a first aperture, and a second tissue engager may have a second aperture. The first aperture may be fitted to the first end of the tube, and the second aperture may be fitted to the second end of the tube. The tube, first tissue engager, and second tissue engager may form an object capable of receiving tissue between the first and second tissue engagers.

In any of the embodiments, the first or second tissue engager may be a disk shaped object.

In any of the embodiments, the first tissue engager may have a different shape from the second tissue engager.

In any of the embodiments, the first tissue engager may be a different size from the second tissue engager.

In any of the embodiments, the first tissue engager and the second tissue engager may include first and second base portions, respectively, and the first and second base portions may be substantially perpendicular to the tube.

In any of the embodiments, the first tissue engager and the second tissue engager may include first and second angled portions, respectively, wherein the first and second angled portions are angled towards each other.

In any of the embodiments, the first and second angled portions may touch each other at least one point between the first and second angled portions.

In any of the embodiments, the first and second tissue engagers may be symmetrical mirror images of each other.

In any of the embodiments, the first and second tissue engagers may be different shapes or sizes.

In any of the embodiments, the tube may be configured to connect at least two cavities within a patient, and the first tissue engager may be positioned within a first cavity and the second tissue engager may be positioned within a second cavity.

In any of the embodiments, the at least one tissue may be held between the first and second tissue engagers with a compressive force.

In any of the embodiments, the compressive force may be configured to cause the tissue to undergo necrosis.

In any of the embodiments, the length of the tube may be at least a thickness of the tissue.

In any of the embodiments, the apparatus may be dissolvable within a patient after a period of time.

In any of the embodiments, the apparatus may include a tracking mechanism.

In any of the embodiments, the second tissue engager may be configured to be deployed within a distal cavity and the first tissue engager may be configured to be deployed within a proximal cavity.

In any of the embodiments, the first tissue engager and the second tissue engager may be at least partially hollow.

In any of the embodiments, at least one of the first tissue engager or the second tissue engager may have a textured portion.

In any of the embodiments, the textured portion may be at a line perpendicular to a central axis of the tube.

In any of the embodiments, the textured portion may be faced away from at least one of the first tissue engager or the second tissue engager.

In any of the embodiments, the textured portion may be faced toward at least one of the first tissue engager or the second tissue engager.

In any of the embodiments, the apparatus may be flexible.

In any of the embodiments, at least one of the tube, the first tissue engager, or the second tissue engager may be elastically deformable.

In any of the embodiments, the apparatus may be elastically compressible.

In any of the embodiments, the first tissue engager and the second tissue engager may be folded away from each other and toward a central axis of the tube producing a folded configuration.

In any of the embodiments, the folded configuration may be adapted for delivery in at least one of a catheter and an endoscope.

In any of the embodiments, the tube may include a one-way valve.

In any of the embodiments, the tube may include a pressure relief valve.

In any of the embodiments, the tube may include a plug, and the plug may prevent material from moving through the tube.

In any of the embodiments, the apparatus may be constructed out of a silicone material.

In any of the embodiments, the apparatus may include a radiopaque material.

In any of the embodiments, the concentration of the radiopaque material may be correlated to a weight of the apparatus.

In any of the embodiments, the radiopaque material may be barium sulfate and the concentration of barium sulfate is approximately 20% of the weight of the apparatus.

According to another embodiment, a method of folding a device may include expanding a central aperture of the device. The central aperture may include a first flexible flange and a second flexible flange. The method may further include extending the first flange and the second flange away from each other and radially compressing the first flange and the second flange into a pleated configuration toward an axis extending through a center of the central aperture. The method may further include compacting the device in the pleated configuration such that a diameter of the device is equal to or less than an inner diameter of a loading mechanism and inserting a tube within the central aperture.

In any of the embodiments, the step of folding a device may include inserting an expanding mechanism into the central aperture. The central aperture may assume an outer shape of the expanding mechanism, and the central aperture may be expanded with the expanding mechanism.

In any of the embodiments, the step of folding a device may include stretching the central aperture around the expanding mechanism. The expanding mechanism may be tapered.

In any of the embodiments, the expanding mechanism may include longitudinal flutes, and the central aperture may at least partially overlap over a portion of the longitudinal flutes.

In any of the embodiments, the step of folding a device may include inserting a centering mandrel into at least one of an expanding mechanism or the tube.

In any of the embodiments, the step of folding a device may include closing an implant compressor over the device and an expanding mechanism along the axis.

In any of the embodiments, the step of folding a device may include compressing lengthwise portions of device corresponding to at least one longitudinal flute along the expanding mechanism.

In any of the embodiments, the step of folding a device may include removing the expanding mechanism from the device. The implant compressor may maintain a compressive force along the axis.

In any of the embodiments, the pleated configuration may be substantially symmetrically around an outer circumference of the device.

In any of the embodiments, the step of folding a device may include inserting a finned mandrel into the central aperture. The finned mandrel may be shaped to correspond with the pleated configuration.

In any of the embodiments, the step of folding a device may include advancing an implant compressor, with the device in the pleated configuration, into an expanded end of a tapered tool and toward a compressed end of the tapered tool. The implant compressor may maintain the pleated configuration.

In any of the embodiments, the tapered tool may be lubricated.

In any of the embodiments, the tapered tool may be at least partially transparent.

In any of the embodiments, the step of folding a device may include advancing the device from the tapered tool into a sheath.

In any of the embodiments, the sheath may be contained in a sheath holder and the tapered tool and the sheath holder may be removably attachable.

In any of the embodiments, the sheath may be contained in a sheath holder and the tapered tool and the sheath holder may be permanently attached.

In any of the embodiments, the step of folding a device may include further compressing the device along the axis into a compressed configuration.

In any of the embodiments, the tube may provide a passageway within the device in the compressed configuration.

In any of the embodiments, the step of folding a device may include advancing the device in the compressed configuration into a sheath. The sheath may maintain the compressed configuration of the device.

In any of the embodiments, the step of folding a device may include lubricating the sheath.

In any of the embodiments, the step of folding a device may include using a rod to insert the tube into the central aperture.

According to yet another embodiment, a method of loading a compressed implant into a delivery device may include aligning a central aperture of the compressed implant with a central lumen of the delivery device, wherein the delivery device may include an implant positioner at least partially within a capsule. The method may further include advancing the implant positioner at least partially out from the capsule toward the compressed implant, inserting a first flange of the compressed implant into the implant positioner, and retracting the implant positioner with the compressed implant into the capsule.

In any of the embodiments, the step of loading a compressed implant may include inserting a centering mandrel through the central aperture of the compressed implant.

In any of the embodiments, the step of loading a compressed implant may include inserting the centering mandrel into the central lumen of the delivery device.

In any of the embodiments, the step of loading a compressed implant may include inserting the compressed implant into a first end of a loading tool.

In any of the embodiments, the loading tool may include multiple detachable components.

In any of the embodiments, the compressed implant may be secured in a compressed configuration within a sheath within the loading tool.

In any of the embodiments, the compressed implant may separate from the sheath as the first flange is advanced into the implant positioner.

In any of the embodiments, the step of loading a compressed implant may include inserting the capsule at least partially into a second end of the loading tool.

In any of the embodiments, the step of loading a compressed implant may include screwing the capsule into the loading tool.

In any of the embodiments, the step of loading a compressed implant may include attaching a loading grip to the delivery device, wherein the loading grip is configured to secure the capsule.

In any of the embodiments, the step of loading a compressed implant may include radially expanding the implant positioner within the loading tool as the implant positioner advances toward the compressed implant.

In any of the embodiments, the step of loading a compressed implant may include pushing the compressed implant into the implant positioner with a pushing mechanism.

In any of the embodiments, the step of loading a compressed implant may include reducing a length of the central lumen of the delivery device to retract the implant positioner.

In any of the embodiments, the step of loading a compressed implant may include controlling the length of the central lumen with a rotating device on a proximal end of the delivery device.

In any of the embodiments, the step of loading a compressed implant may include uncoupling the loading tool from the delivery device.

In any of the embodiments, the step of loading a compressed implant may include collapsing the implant positioner around at least the first flange of the compressed implant.

In any of the embodiments, the second flange may not be secured by the implant positioner.

In any of the embodiments, the compressed implant may be substantially retracted within the capsule.

In any of the embodiments, a tube within the central aperture of the compressed implant may be advanced into the capsule with the compressed implant.

According to yet another embodiment, a method of packaging and deploying an implant may include extending the implant along a lengthwise direction of the implant, radially compressing the implant along the lengthwise direction, and inserting a first flange of the implant into an implant holder on an end of a deployment device. The method may further include retracting the implant holder and implant into the deployment device, advancing the implant holder toward the end of the delivery device, and releasing a second flange out from the end of the deployment device. The method may further include advancing the implant holder at least partially out from the end and releasing the first flange from the deployment device and the implant holder.

In any of the embodiments, the step of packaging and deploying an implant may include extending the first flange and the second flange away from each other.

In any of the embodiments, the step of packaging and deploying an implant may include expanding a central aperture of the implant.

In any of the embodiments, the step of packaging and deploying an implant may include supporting a folded shape of the implant with a finned mandrel.

In any of the embodiments, the step of packaging and deploying an implant may include inserting a tube within the implant.

In any of the embodiments, the step of packaging and deploying an implant may include inserting the implant into a sheath. The sheath may maintain a compressed configuration of the implant.

In any of the embodiments, the step of packaging and deploying an implant may include inserting a centering mandrel through at least one of the implant and the deployment device.

In any of the embodiments, the step of packaging and deploying an implant may include extending the implant holder from the deployment device.

In any of the embodiments, the step of packaging and deploying an implant may include radially expanding the implant holder.

According to still another embodiment, a method of deploying a device within a patient may include delivering the device in a compressed configuration with a deployment mechanism at least partially through a first opening to a first cavity within the patient and releasing a second flange of the device within the first cavity. The method may further include retracting the deployment mechanism back through the first opening, wherein a first flange of the device is locatable within a second cavity and releasing the first flange of the device within the second cavity.

In any of the embodiments, the step of deploying a device may include retaining at least one tissue of the patient between the first flange and the second flange.

In any of the embodiments, the step of deploying a device may include preventing circulation from flowing within a retained portion of the at least one tissue.

In any of the embodiments, the step of deploying a device may include causing necrosis to occur within the portion of the at least one tissue.

In any of the embodiments, the step of deploying a device may include causing a continuous region of necrosed tissue within the portion. The necrosed tissue and the device may be configured to detach from the at least one tissue.

In any of the embodiments, the step of deploying a device may include tracking the position of the device after the device has detached from the at least one tissue.

In any of the embodiments, the device may include at least one textured portion configured to prevent circulation within the at least one tissue.

In any of the embodiments, the step of deploying a device may include allowing material to move in at least one direction through the device.

In any of the embodiments, the device may include a valve.

In any of the embodiments, the step of deploying a device may include expanding the first cavity within the patient with an expanding mechanism.

In any of the embodiments, the expanding mechanism may be a balloon.

In any of the embodiments, the expanding mechanism may be a guidewire with a bent region.

In any of the embodiments, the step of deploying a device may include pulling tissue walls of the first and second cavities together with the deployment mechanism and the expanding mechanism. A tissue wall of the first and second cavities may be compressed between the deployment mechanism and the expanding mechanism.

In any of the embodiments, the step of deploying a device may include anchoring the deployment mechanism within the patient with the expanding mechanism.

In any of the embodiments, the step of deploying a device may include dilating at least one of the first opening of the first cavity and a second opening of the second cavity with a dilation balloon.

In any of the embodiments, the step of deploying a device may include rotating a portion of the deployment mechanism to atraumatically secure the first cavity and the second cavity.

In any of the embodiments, the step of deploying a device may include atraumatically aligning the first opening of the first cavity to a second opening of the second cavity.

In any of the embodiments, the step of deploying a device may include threading the second opening and the first opening over a threaded leading edge of the deployment device.

According to another embodiment, a method of deploying an implant from a delivery device may include advancing an implant positioner toward an end of the delivery device, wherein the implant maybe secured in a compressed configuration within the implant holder. The method may further include releasing a second flange of the implant through the end, advancing the implant holder at least partially out of the end, and releasing a first flange of the implant from the implant positioner.

In any of the embodiments, a position of the implant positioner may be controlled by a length of a central lumen of the delivery device.

In any of the embodiments, the central lumen may be a coil.

In any of the embodiments, the length of the central lumen may be controlled with a rotating device on the delivery device.

In any of the embodiments, the implant positioner may radially expand out of the end due to a spring force of the implant positioner.

According to yet another embodiment, a delivery device configured to deploy a shunt within a patient may include a generally cylindrical container configured to retain the shunt in a compressed configuration and a positioning mechanism movable between a first position and a second position within the container. The positioning mechanism may be within the container in the first position and may be at least partially extended beyond a distal end of the container in the second position. The delivery device may further include a control device configured to move the positioning mechanism between the first position and the second position and a lumen connecting a proximal end of the container to the control device and extendable into the patient. The control device may manipulate the positioning mechanism through the lumen.

In any of the embodiments, a guidewire with a distal end may be configured to be inserted into a distal cavity of the patient. At least a portion of the distal end may automatically bend into at least one coil.

In any of the embodiments, the at least one coil may be three-dimensional shape.

In any of the embodiments, an expandable mechanism may be on a distal end of the lumen.

In any of the embodiments, the expandable mechanism may be a balloon.

In any of the embodiments, the balloon may be inflatable through the lumen with at least one of gas or liquid.

In any of the embodiments, the control device may control a position and an amount of expansion of the expandable mechanism.

In any of the embodiments, the distal end of the container may be threaded.

In any of the embodiments, rotating the container proximal to at least one tissue within the patient atraumatically may secure the at least one tissue over the distal end of the capsule.

In any of the embodiments, the distal end of the container may have a hook.

In any of the embodiments, the positioning mechanism may be configured to secure a flange of the shunt in the first position and release the flange in the second position.

In any of the embodiments, the positioning mechanism may include at least one finger around a perimeter of the positioning mechanism. A flange of the shunt may be securable within the at least one finger.

In any of the embodiments, the at least one finger may be constrained by the container in the first position.

In any of the embodiments, a spring force of the at least one finger may be less than a spring force of the flange.

In any of the embodiments, the at least one finger may radially expand from the first position to the second position.

In any of the embodiments, the lumen may transmit at least one force from the control device to the positioning mechanism.

In any of the embodiments, the lumen may include a coiled tube.

In any of the embodiments, the lumen may include a threaded guide.

In any of the embodiments, the control device may control at least one of a position or a rotation of the container.

In any of the embodiments, the control device may include a locking mechanism.

According to still another embodiment, a loading device for folding and loading a device into a delivery device may include a dilator configured to expand a lumen of the device, wherein the lumen defines a central axis of the device and a compressor configured to radially compress the device along a central axis. The device may assume a pleated configuration with the compressor. The loading device may further include a loading tool configured to receive the implant in the pleated configuration and radially compress the implant into a compressed configuration. The loading tool may be attachable and alignable with the delivery device.

In any of the embodiments, the dilator may be tapered.

In any of the embodiments, the dilator may have longitudinal flutes along a central axis of the dilator.

In any of the embodiments, the compressor may have at least one movable rod configured to radially close on the implant.

In any of the embodiments, a sliding ring may be movable along a length of the compressor. A position of the at least one movable rods with respect to a central axis of the compressor may be dependent on a position of the sliding ring along the length of the compressor.

In any of the embodiments, the loading tool may include three separate and attachable sections.

In any of the embodiments, the loading tool may be one continuous and hollow lumen.

In any of the embodiments, the loading tool may be substantially hollow along a central axis of the loading tool.

In any of the embodiments, the device may be movable along the central axis of the loading tool.

In any of the embodiments, the loading tool may have a tapered section with at least one flute along a central axis of the loading tool.

In any of the embodiments, the loading tool may have a threaded end attachable with an end of the delivery device.

In any of the embodiments, a rod may be configured to push the device within the loading tool.

In any of the embodiments, a centering rod may be configured to be inserted along the central axis of at least one of the device, the dilator, the compressor, or the loading tool.

1. A device for implantation in a body, the device comprising:
a body defining a lumen, the body having a first end and a second end, the lumen defining a central axis, the body comprising:
a first flexible flange comprising a first base portion and a first angled portion, the first base portion being substantially perpendicular to the central axis and extending radially away from the first end; and
a second flexible flange comprising a second base portion and a second angled portion, the second base portion being substantially perpendicular to the central axis and extending radially away from the second end,
wherein the first and second angle portions are substantially angled toward each other.

2. The device of claim 1, wherein the first and second angled portions have a narrower gap between them than the gap between the first and second base portions.

3. (canceled)

4. The device of claim 1, wherein the first and second flanges are symmetrical mirror images of each other.

5. The device of claim 1, wherein the first and second flanges are different shapes or sizes.

6. The device of claim 1, wherein the first flange and the second flange are configured to fit at least one tissue in a patient between the first flange and the second flange.

7. The device of claim 6, wherein the lumen is configured to connect at least two cavities within the patient, wherein the first flange is positioned within a first cavity and the second flange is positioned within a second cavity.

8. The device of claim 6, wherein the at least one tissue is held between the first and second flanges with a compressive force.

9. (canceled)

10. The device of claim 8, wherein the compressive force is configured to cause the at least one tissue to undergo necrosis.

11. (canceled)

12. The device of claim 1, wherein the device is dissolvable within a patient after a period of time.

13. (canceled)

14. (canceled)

15. The device of claim 1, wherein the first flange and the second flange are at least partially hollow.

16. The device of claim 1, wherein at least one of the first flange or the second flange has a textured portion that includes at least one of a groove, a bump, and/or a line perpendicular to the central axis.

17.-19. (canceled)

20. The device of claim 16, wherein the textured portion is faced away from at least one of the first flange or the second flange.

21. The device of claim 16, wherein the textured portion is faced toward at least one of the first flange or the second flange.

22. (canceled)

23. The device of claim 1, wherein at least one of the body, first flange and second flange are elastically deformable.

24. (canceled)

25. The device of claim 1, wherein the first flange and the second flange are foldable away from each other and toward the central axis, producing a folded configuration.

26. The device of claim 25, wherein the folded configuration is adapted for delivery in at least one of a catheter or an endoscope.

27. The device of claim 1, wherein the lumen includes at least one of a one-way valve, a pressure relief valve, and/or a plug, wherein the plug prevents material from moving through the lumen.

28.-35. (canceled)

36. A medical apparatus for forming a shunt between two tissues, the apparatus comprising:
a tube having a first end and a second end;
a first tissue engager having a first aperture, the first aperture fitted to the first end of the tube;
a second tissue engager having a second aperture, the second aperture fitted to the second end of the tube;
wherein the tube, first tissue engager and second tissue engager form an object capable of receiving tissue between the first and second tissue engagers.

37. The medical apparatus of claim 36, wherein the first or second tissue engager is a disk shaped object.

38. (canceled)

39. (canceled)

40. The medical apparatus of claim 36, wherein the first tissue engager and the second tissue engager comprise first and second base portions, respectively, wherein the first and second base portions are substantially perpendicular to the tube, and wherein the first tissue engager and the second tissue engager comprise first and second angled portions, respectively, wherein the first and second angled portions are angled towards each other.

41.-178. (canceled)
